(19) United States
(12) Patent Application Publication
(10) Pub. No.: US 2016/0115192 A1
(43) Pub. Date: Apr. 28, 2016

(54) ANTIOXIDANT, ANTI-INFLAMMATORY AND ANTICANCER DERIVATIVES OF TRIPTOLIDE AND NANOSPHERES THEREOF

(71) Applicant: CEDARS-SINAI MEDICAL CENTER, Los Angeles, CA (US)

(72) Inventors: John S. Yu, Los Angeles, CA (US); Bong Seop Lee, Torrance, CA (US); Lei Zhang, Los Angeles, CA (US)

(73) Assignee: Cedars-Sinai Medical Center, Los Angeles, CA (US)

(21) Appl. No.: 14/895,421

(22) PCT Filed: Jun. 10, 2014
(86) PCT No.: PCT/US14/41752

§ 371 Date: Dec. 2, 2015

Related U.S. Application Data
(60) Provisional application No. 61/833,132, filed on Jun. 10, 2013.

(51) Int. Cl. C07J 73/00; A61K 49/00; A61K 9/14

(52) U.S. Cl. CPC C07J 73/003; A61K 9/14; A61K 49/0002

(57) ABSTRACT

This invention relates to uses of conjugates of triptolide nanoprodrugs in cancer immunotherapy, specifically compound such as D-I or D-II wherein X₁ is a antioxidant, an anti-inflammatory or an anti-cancer agent and nanospheres thereof, and A is be selected from the group consisting of branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic groups.

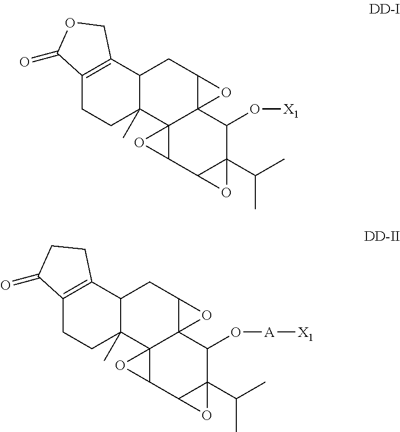

### CROSS-REFERENCE TO RELATED APPLICATIONS

This application claims the benefit under 35 U.S.C. §119(e) of U.S. Provisional Application No. 61/833,132, filed Jun. 10, 2013, the content of which is incorporated herein by reference in its entirety.

### FIELD OF INVENTION

This invention relates to uses of conjugates of triptolide nanoprodrugs in cancer immunotherapy, specifically antioxidant, anti-inflammatory, and anticancer derivatives of triptolide and nanospheres thereof.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### Cancer Immunotherapy and Anti-Immunosuppressive Effect of Triptolide

Suppression of anticancer immunity is one the major mechanisms by which cancer cells escape host anticancer immune defense. B7-H1 is a member of the B7 family of costimulatory molecule involved in the prevention of autoimmune response of the immune system and anticancer immune response. B7-H1 is overexpressed in a variety of human cancers. B7-H1 makes tumors resistant to antitumor immunity of the body and anticancer immunotherapy. The overexpressed B7-H1 interacts with programmed death receptor (PD-1) on activated T cells, leading to deaths of tumor-killing T cells. This makes cancer cells resistant against T cell mediated anticancer immunity. It has been suggested that blockage of B7-H1 and/or PD-1 improves therapeutic efficacy of cancer immunity of the body and also cancer immunotherapy. The attempts to overcome the B7-H1/PD-1-related immunosuppression have been confined to the development of antibodies against B7-H1 or PD-1. However, the antibodies block all B7-H1, resulting in side effects of impairing autoimmune protection via B7-H1. Triptolide is known to inhibit the expression of B7-H1 by cancer cells. Therefore, triptolide can be used as adjuvant therapeutic to enhance the anticancer immunity Triptolide can be used together with conventional chemotherapeutics and/or along with cancer immunotherapy. The new nanoprodrug technology of this invention combines targeted antioxidant, anti-inflammatory, and anticancer therapy with cancer immunotherapy. In the present invention, design and synthesis of anti-immunosuppressive triptolide derivatives and methods of preparation nanometer-sized prodrugs (nanoprodrug), and methods of treating malignancies are described. The inventive triptolide prodrugs and nanoprodrugs thereof combine targeted antioxidant, anti-inflammatory, and anticancer therapy with cancer immunotherapy. There is no mAb-associated side effect because the new technology uses highly tumor-selective nanoprodrugs that accumulate and are activated in the tumor tissues. In the present invention, we disclose a small molecule inhibitor of B7-H1 expression in cancer cells and its formation into nanoprodrug.

### Antineoplastic Effect of Camptothecin

Camptothecin is a plant alkaloid first isolated from the wood and bark of Camptotheca acuminate (Nyssaceae), and exhibits its antineoplastic effect by the inhibition of DNA relaxation by DNA topoisomerase I. However, camptothecin is essentially insoluble in water, and therefore, numerous derivatives have been developed to increase the water solubility (Thomas et al., Camptothecin: Current perspectives. BIOORG. MED. CHEM., 12, 2004, 1585-1604: Pizzolato et al., The Camptothecin. THE LANCET, 361, 2003, 2235-2242).

Camptothecin consists of a pentacyclic structure having a lactone in the E-ring, which is essential for antitumor effects of the molecule. It has been demonstrated that the main transformation and elimination pathways of the drug comprise lactone hydrolysis and urinary excretion. In fact, the lactone form is 50% hydrolyzed to an open ring 30 minutes after administration. The sodium salt showed a lower activity than camptothecin, because at pH 7.4 the inactive form (open ring) predominates on the lactone active form (closed ring).

### Antioxidant Effect of α-Lipoic Acid

Molecules containing a dithiolane moiety are widely investigated due to their antioxidant properties. α-Lipoic acid (thioctic acid, 1,2-dithiolane-3-pentanoic acid), which has dithiolane ring in its molecule, is a widely distributed natural substance which was originally discovered as a growth factor. Physiologically, it acts as a coenzyme of the oxidative decarboxylation of α-keto carboxylic acid (e.g., pyruvates) and as an antioxidant, and it is able to regenerate vitamin C, vitamin E, glutathione and coenzyme Q10. In pathological conditions, lipoic acid is applied in the treatment of diabetic polyneuropathy, liver cirrhosis and metal intoxications.

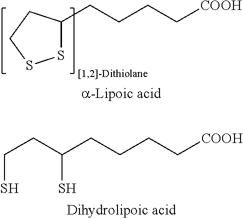

Lipoic acid and dihydrolipoic acid are capable of trapping a number of radicals both in a lipid and in an aqueous environment. Lipoic acid and dihydrolipoic acid act as antioxidants not only by direct radical trapping and/or metal chelation but also by recycling other antioxidants (e.g., vitamin C, vitamin E) and by reducing glutathione, which in turn recycles vitamin E. The two thiol groups present in [1,2]-dithiolane ring system confer it a unique antioxidant potential. The disulfides with a cyclic five-member ring such as lipoic acid have been found to be more effective in reductive and/or nucleophilic attack than open-chain derivatives such as cystine or glutathione.

The antioxidant potential of a compound can be evaluated based on the properties such as (1) specificity of free radical scavenging, (2) interaction with other antioxidants, (3) metal-chelating activity, (4) effects on gene expression, (5) absorption and bioavailability, (6) location (in aqueous or membrane domains, or both), and (7) ability to repair oxidative damage (Packer et al., FREE RADICAL BIOLOGY & MEDICINE. 19(2):227-250, 1995). According to the above criteria, the [1,2]-dithiolane containing lipoic acid/dihydrolipoic acid redox system has been regarded as a universal antioxidant.

There have been many attempts to develop lipoic acid derivatives or complexes having antioxidant activity. U.S. Pat. Nos. 6,090,842; 6,013,663; 6,117,899; 6,127,394; 6,150,358; 6,204,288, 6,235,772; 6,288,106; 6,353,011; 6,369,098; 6,387,945; 6,605,637; 6,887,891; 6,900,338; and 6,936,715 are some examples.

In many other U.S. patents, the natural and synthetic lipoic acid derivatives and their metabolites are disclosed for use in preventing skin aging and in the treatment of free radical mediated diseases, including inflammatory, proliferative, neurodegenerative, metabolic and infectious diseases.

### Inhibitory Activity on NO-Synthase and Trapping the Reactive Oxygen Species (ROS)

Various conditions or disease conditions have demonstrated a potential role of nitric oxide (NO) and the ROS's and the metabolism of glutathione in their physiopathology.

Conditions or disease conditions where nitrogen monoxide and the metabolism of glutathione as well as the redox status of thiol groups are involved include but are not limited to: cardiovascular and cerebrovascular disorders (e.g., atherosclerosis, migraine, arterial hypertension, septic shock, ischemic or hemorrhagic cardiac or cerebral infarctions, ischemias and thromboses); disorders of the central or peripheral nervous system (e.g., neurodegenerative nervous system); neurodegenerative diseases including cerebral infarctions, sub-arachnoid hemorrhaging, ageing, senile dementias (e.g., Alzheimer's disease), Huntington's chorea, Parkinson's disease, prion disease (e.g., Creutzfeld Jacob disease), amyotrophic lateral sclerosis, pain, cerebral and spinal cord traumas; proliferative and inflammatory diseases (e.g., atherosclerosis), amyloidoses, and inflammations of the gastro-intestinal system; organ transplantation; diabetes and its complications (e.g., retinopathies, nephropathies and polyneuropathies, multiple sclerosis, myopathies); cancer; autosomal genetic diseases (e.g., Unverricht-Lundborg disease); neurological diseases associated with intoxications (e.g., cadmium poisoning, inhalation of n-hexane, pesticides, herbicides), associated with treatments (e.g., radiotherapy) or disorders of genetic origin (e.g., Wilson's disease); and impotence linked to diabetes.

These conditions and disease conditions are characterized by an excessive production or a dysfunction of nitrogen monoxide and/or the metabolism of glutathione and of the redox status of the thiol groups (Duncan and Heales, Nitric Oxide and Neurological Disorders, MOLECULAR ASPECTS OF MEDICINE. 26:67-96, 2005; Kerwin et al., Nitric Oxide: A New Paradigm For Second Messengers, J. MED. CHEM. 38:4343-4362, 1995; Packer et al., FREE RADICAL BIOLOGY & MEDICINE. 19:227-250, 1995). U.S. Pat. Nos. 6,605,637, 6,887,891, and 6,936,715 disclose that lipoic acid derivatives inhibit the activity of NO-synthase enzymes producing nitrogen monoxide NO and regenerate endogenous antioxidants which trap the ROS and which intervene in a more general fashion in the redox status of thiol groups. U.S. Pat. Nos. 5,693,664, 5,948,810, and 6,884,420 disclose the use of racemic α-lipoic acid or their metabolites, salts, amides or esters for the synthesis of drugs for the treatment of diabetes mellitus of types I and II. U.S. Pat. No. 5,925,668 discloses a method of treating free radical mediated diseases, and/or reducing the symptoms associated with such diseases whereby the compounds with antioxidant activity contain 1,2-dithiolane, reduced or oxidized forms. U.S. Pat. No. 6,251,935 discloses methods for the prevention or treatment of migraine comprising the administration of an active ingredient selected from the group consisting of racemic alpha-lipoic acid, enantiomers and pharmaceutically acceptable salts, amides, esters or thioesters thereof. U.S. Pat. Nos. 6,472,432 and 6,586,472 disclose the treatment of a chronic inflammatory disorder rosacea by application of a composition containing lipoic acid and/or lipoic acid derivatives. There is also strong evidence that the neuroprotective effects of lipoic acid and dihydrolipoic acid are mediated by antioxidant and free radical scavenging mechanisms (Packer et al., FREE RADICAL BIOLOGY & MEDICINE. 22:359-378, 1997).

### Non-Steroidal Anti-Inflammatory Drugs

Non-steroidal anti-inflammatory drugs (NSAIDs) are widely used in the treatment of pain, fever, and inflammation. The major mechanism by which NSAIDs exert their anti-inflammatory activity is the inhibition of cyclooxygenase-derived prostaglandin synthesis, which is also responsible for adverse side effects, such as irritation and ulceration of the gastrointestinal (GI) mucosa (Whittle, 2003). There are two types of COX enzymes, namely COX-1 and COX-2. COX-1 is expressed constitutively in many tissues, whereas COX-2 is expressed only at the site of inflammation (S. Kargan et al. GASTROENTEROL., 111: 445-454, 1996). The prostaglandins whose production is mediated by COX-1 are responsible for the maintenance of gastric mucosal integrity. Thus, the GI side effects are generally believed to result from the combined effect of the irritation caused by the free carboxylic groups in NSAIDs and blockage of prostaglandin biosynthesis in the GI tract (Dannhardt and Kiefer, 2001). In addition to the side effect which is attributed to their inhibitory effect on the activity of cyclooxygenase, the acidic moiety of these NSAIDs also contributes to the gastrointestinal side effect observed in response to these drugs (Tammara et al., 1993).

Epidemiologic studies have documented that a subset of NSAIDs decrease the risk for Alzheimer's disease (AD). The efficacy of NSAIDs in AD might be attributable to either anti-inflammatory or anti-amyloidogenic activities. It has been reported that ibuprofen, indomethacin and sulindac sulphide decrease the highly amyloidogenic A1342 peptide independently of COX activity (NATURE, 414:212-216 (2001)).

NSAIDs have also been shown to inhibit angiogenesis through direct effects on endothelial cells.

Although inflammatory oxidant hypochlorous acid (HOCl) generated by the myeloperoxidase (MPO)-H2O2/Cl— system comprises an important mechanism of host defense against infection, the overproduction and extracellularly generated HOCl is cytotoxic and is believed to be implicated in the pathogenesis of numerous diseases including neurodegenerative disorders, atherosclerosis, chronic inflammatory conditions, and cancer (Malle et al., BR J PHARMACOL 2007: 1-17).

Hypochlorous acid is a powerful oxidizing agent that can react with many biological molecules. In the presence of physiological concentration of chloride ions, H2O2 is efficiently halogenated by the heme enzyme MPO to yield hypochlorous acid, by far the most abundant oxidant generated by activated phagocyte cells (Krasowska et al., BRAIN RES. 997:176-184 (2004)). Hypochlorous acid can chlorinate cytosolic proteins and nuclear DNA bases and induce lipid peroxidation in phospholipid and lipoprotein (Spickett C M., PHARMACOL THERAPEUTICS 115:400-409 (2007)). Importantly, the damages caused by HOCl to the intracellular glutathione and protein thiols are irreversible and can be replaced only by resynthesis (Dalle-Donne et al., FREE RADIC BIOL MED 32(9):927-937 (2002)). Furthermore, HOCl can be converted into damaging hydroxyl radicals (Candeias et al., FEBS LETT 333(1,2):151-153 (1993)). Most NSAIDs are able to scavenge hypochlorous acid in the aqueous environment and some NSAIDs inhibit the MPO by direct interaction with the enzyme (Neve et al., EUROPEAN J PHARMACOL 417:37-43 (2001)).

### Anticancer Effects of NSAIDs

A number of epidemiologic studies, clinical trials, and animal studies have shown that NSAIDs can be effective in the prevention and treatment of certain cancers. (Keller et al., Chemoprevention strategies using NSAIDs and COX-2 inhibitors. CANCER BIOL THER (2003) 2:S140-9; Gupta et al., Colorectal cancer prevention and treatment by inhibition of cyclooxygenase-2. NAT REV CANCER (2001) 1:11-21; Umar et al., Development of COX inhibitors in cancer prevention and therapy. AM J CLIN ONCOL (2003) 26:S48-57; Harris et al., Aspirin, ibuprofen, and other non-steroidal anti-inflammatory drugs in cancer prevention: a critical review of nonselective COX-2 blockade [review]. ONCOL REP 2005; 13: 559-83). It has also been suggested that the long term use of certain NSAIDs reduces the risk of colorectal, breast, and ovarian cancer. Taketo et al., Cyclooxygenase-2 inhibitors in tumorigenesis. J NATL CANCER NST (1998) 90:1529-36; Sandler et al. A randomized trial of aspirin to prevent colorectal adenomas. N ENGL J MED (2003) 348:891-9; Saji et al. Novel sensitizing agents: potential contribution of COX-2 inhibitor for endocrine therapy of breast cancer. BREAST CANCER (2004) 11:129-33.

The molecular mechanisms by which NSAIDs exhibit antineoplastic effects are poorly understood and a matter of intensive investigation. The chemopreventive and antitumorigenic effects of NSAIDs are partially attributed to the induction of apoptosis followed by inhibition of COX-2. Lin et al., The role of cyclooxygenase-2 inhibition for the prevention and treatment of prostate carcinoma. CLIN PROSTATE CANCER (2003) 2:119-26; Mann et al., Cyclooxygenase-2 and gastrointestinal cancer. CANCER J (2004) 10:145-52; Basler et al., Nonsteroidal anti-inflammatory drugs and cyclooxygenase-2 selective inhibitors for prostate cancer chemoprevention. J UROL 2004; 171: S59-62; discussion S62-53; Sabichi et al., COX-2 inhibitors and other nonsteroidal anti-inflammatory drugs in genitourinary cancer. SEMIN ONCOL 2004; 31:36-44.

Various studies have also suggested that a COX-2-independent mechanism can also be involved because apoptosis induction by NSAIDs does not always correlate with their ability to inhibit COX-2. Chuang et al., COX-2 inhibition is neither necessary nor sufficient for celecoxib to suppress tumor cell proliferation and focus formation in vitro. MOL CANCER (2008) 7:38; Marx et al., J. Cancer research; Anti-inflammatories inhibit cancer growth—but how? SCIENCE 2001; 291:581-2; Elder et al., Induction of apoptotic cell death in human colorectal carcinoma cell lines by a cyclooxygenase-2 (COX-2)-selective nonsteroidal anti-inflammatory drug: independence from COX-2 protein expression. CARCINOGENESIS (2001) 22:17-25; Jiang et al., Subtraction hybridization identifies a novel melanoma differentiation associated gene, mda-7, modulated during human melanoma differentiation, growth and progression. ONCOGENE (1995) 11:2477-86.

### Neuroprotective and Neurorestorative Effects of Statins

Statins are cholesterol biosynthesis inhibitors used for lowering cholesterol level. Statins also show neuroprotective and neurorestorative benefits in animal models of traumatic brain injury (TBI) and stroke (Chen et al., Ann Neurol 53(6), 743-751, 2003; Jessberger et al., Learn Mem 16(2), 147-154, 2009; Chen et al., Life Sci 81(4), 288-298, 2007; Chen et al., J Cereb Blood Flow Metab 25(2), 281-290, 2005; Lu et al., J Neurotrauma, 21(1), 21-32, 2004; Lu et al., J Neurosurg, 101 (5):813-821, 2004. Wu et al., J Neurosurg, 109(4):691-698, 2008). Traumatic brain injury caused by stroke and trauma is a major health problem worldwide. Ischemia also plays an important role in pathogenesis of TBI. Statins enhance functional recovery after TBI, significantly reduce the neurological functional deficits, and increase neuronal survival (Chen et al., Ann Neurol, 53(6), 743-751, 2003; Lu et al., J Neurotrauma, 24(7): 1132-1146, 2007; Wang et al., Exp Neurol, 206(1), 59-69, 2007).

### Anticancer Effects of Statin

Statins have also antiproliferative effects on various tumor types including breast cancer, acute myloid leukemia, myeloma, prostate cancer, lung cancer, pancreatic cancer, ovarian cancer, cervical cancer, colorectal cancer, and head & neck cancers. [Wong et al., Leukemia 2002; 16: 508-519; Fritz G. Curr Cancer Drug Targets 2009; 9: 626-638; Jakobisiak M, Golab J. Med Res Rev 2010; 30: 102-135; Sassano A, Platanias L C. Cancer Lett 2008; 260: 11-19.]. It has been shown that fluvastatin and simvastatin significantly inhibited the proliferation of MCF-7 breast cancer cell and MDA-MB-231 triplem negative breast cancer cell. [Campbell et al., Cancer Res. 66 (17), 8707-8714, 2006; Kotamraju et al., Cancer Res. 67 (15), 7386-8973, 2007]

### SUMMARY OF THE INVENTION

The following embodiments and aspects thereof are described and illustrated in conjunction with compositions and methods which are meant to be exemplary and illustrative, not limiting in scope.

The present invention relates to novel triptolide (TPL) derivatives. In some embodiments, the triptolide derivatives result from the conjugation of triptolide with a chemotherapeutic agent, an antioxidant, or an anti-inflammatory agent. In some embodiments, the TPL derivatives are: TPL-chemotherapeutic agents, TPL-NSAID, TPL-statin, and TPL-alpha lipoic acid.

The present invention also relates to nanospheres comprising a TPL derivative of the present invention. In various embodiments, the nanosphere comprises a compound of Formula D-I, Formula D-II, or any combinations thereof.

In various embodiments, the nanosphere further comprises an antioxidant. In some embodiments, the antioxidant can be conjugated to a hydrophilic spacer, a hydrophobic spacer, an amphiphilic spacer, or an amphiphilic polymer. In some embodiments, the antioxidant is tocopherol or a derivative or analogue thereof. In some embodiments, the antioxidant can be glutathione, a hydrophobic derivative of glutathione, N-acetyl cysteine, or a hydrophobic derivative of N-acetyl glutathione.

In various embodiments, the nanosphere further comprises a therapeutic agent. In certain embodiments, the delivery of the nanosphere further comprising the therapeutic agent delivers the therapeutic agent to a tumor or cancer. In various embodiments, the delivery of the therapeutic agent to the tumor or cancer treats cancer.

In some embodiments, the therapeutic agent is conjugated to a hydrophilic spacer, a hydrophobic spacer, an amphiphilic spacer, or an amphiphilic polymer. In some embodiments, the therapeutic agent is selected from the group consisting of: chemotherapeutic agent, a statin, nonsteroidal anti-inflammatory drug (NSAID), erythropoietin, peptide, antisense nucleic acid, DNA, RNA, protein, and combinations thereof.

In various embodiments, the nanosphere further comprises an imaging agent. In some embodiments, the imaging agent is conjugated to a hydrophilic spacer, a hydrophobic spacer, an amphiphilic spacer, or an amphiphilic polymer. In some embodiments, the imaging agent is selected from the group consisting of fluorescent molecules, radioisotopes, nucleotide chromophores, enzymes, substrates, chemiluminescent moieties, bioluminescent moieties, and any combinations thereof. In some embodiments, the imaging agent is a fluorophore.

In various embodiments, the nanosphere can further comprise an amphiphilic spacer. In some embodiments, the amphiphilic spacer comprises a chemically active group selected from the group consisting of thiol, amine, carboxylic acid, carboxylic acid NHS ester, maleimide, hydrazine, ketone, aledehyde, and combinations thereof In some embodiments, the amphiphilic spacer is an alkylthiol or an alkylamine. One exemplary alkylthio is 1-octadecanethiol.

In various embodiments, the nanosphere can further comprise a polymer. In various embodiments, the polymer can be selected from the group consisting of a hydrophobic polymer, amphiphilic polymer, and hydrophobically modified hydrophilic polymer. In other embodiments, the polymer can be selected from the group consisting of a polyanhydride, polyester, polyorthoester, polyesteramide, polyacetal, polyketal, polycarbonate, polyphosphoester, polyphosphazene, polyvinylpyrrolidone, polydioxanone, poly(malic acid), poly(amino acid), polymer of N-2-(hydroxypropyl)methacrylamide (HPMA), polymer of N-isopropyl acrylamide (NIPAAm), polyglycolide, polylactide, copolymer of glycolide and lactide (e.g., poly(lactide-co-glycolide), and combinations thereof. In some embodiments, the polymer is poly(lactide-co-glycolide) (PLGA).

In various embodiments, the polymer can contain a side group selected from the group consisting of a hydrophobic molecule, hydrophilic molecule, and amphiphilic molecule. In various embodiments, the side group can be a therapeutic or diagnostic agent. In other embodiments, the therapeutic agent can be selected from the group consisting of a peptide, antisense nucleic acid, and protein. In additional embodiments, the polymer can contain a hydrophobic side groups selected from the group consisting of an aromatic group, amino acid alkyl ester, and aliphatic group.

In various embodiments, the nanosphere further comprises a compound of Formula A-IV, A-V, or any combinations thereof.

The present invention provides for methods of using nanospheres formed from the molecules as described herein.

Various embodiments of the present invention provide for a method of treating cancer in a subject in need thereof, comprising: providing a nanosphere comprising a compound of the invention Formula D-I, Formula D-II or combinations thereof; and administering a therapeutically effective amount of the nanosphere to the subject to treat the cancer.

The present invention also provides a method of treating cancer in a subject in need thereof, comprising: providing a nanosphere of the present invention; and administering a therapeutically effective amount of the nanosphere to the subject to treat the cancer.

In one aspect, the disclosure provides a method for delivering a nanosphere to a tumor or cancer tissue in a subject, comprising administering a therapeutically effective amount of a nanosphere to the subject, wherein the nanosphere comprises a compound selected from Formula D-I, Formula D-II, and any combinations thereof

In another aspect, the disclosure provides a method for detecting or diagnosing cancer in a subject in need thereof comprising: providing a nanosphere comprising a compound of Formula D-I, Formula D-II, or any combinations thereof, and an imaging agent; administering an effective amount of the nanosphere to the subject; and imaging the subject to detect or diagnose the cancerI. In some embodiments, the imaging agent can be conjugated to a hydrophilic spacer, a hydrophobic spacer, an amphiphilic spacer, or an amphiphilic spacer.

The present invention also provides a pharmaceutical composition, comprising a compound of the invention and pharmaceutically acceptable carrier or excipient.

The present invention also provides a pharmaceutical composition, comprising a nanosphere of the invention and pharmaceutically acceptable carrier or excipient.

Other features and advantages of the invention will become apparent from the following detailed description, taken in conjunction with the accompanying drawings, which illustrate, by way of example, various features of embodiments of the invention.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 illustrates the synthesis of hydrophobic derivative of triptolide TPL-ALA (A) and preparation of nanoprodrug of triptolide using spontaneous nanoemulsification (B).

FIG. 2 illustrates oxidation and degradation of triptolide prodrug.

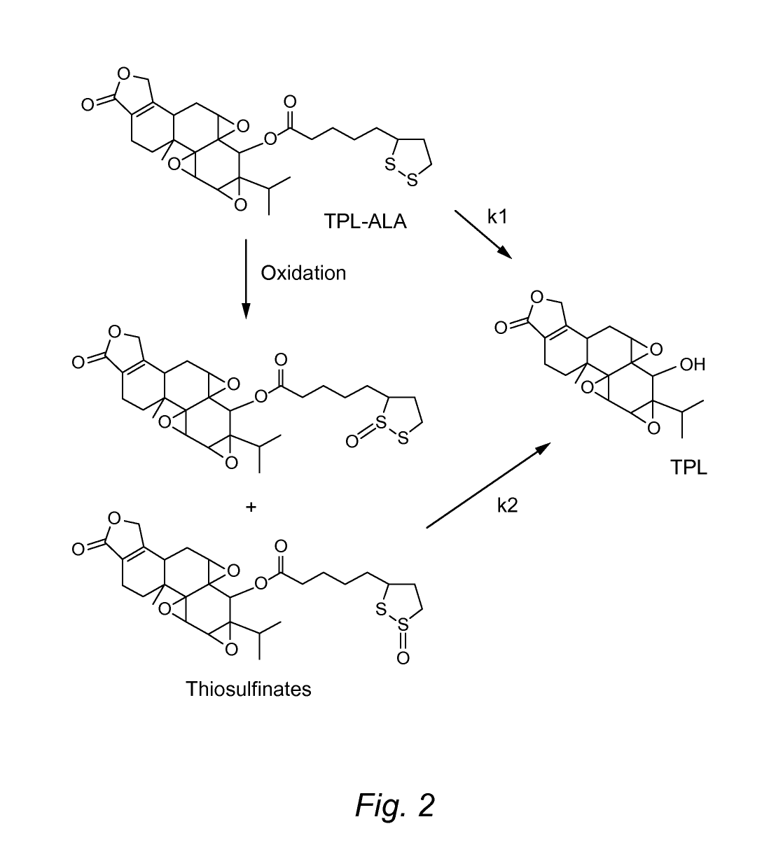

FIG. 3 depicts the oxidation of TPL prodrug in nanoprodug. P1: oxidized TPL prodrug, P2: intact TPL prodrug.

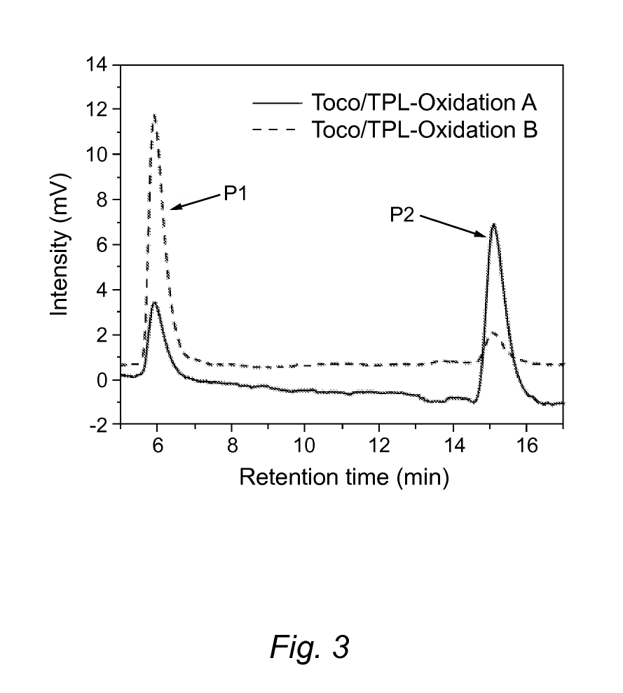

FIG. 4 depicts the enzymatic degradation of TPL prodrug in nanoprodug. The nanoprodrug was enzymatically hydrolyzed in the presence of 5 U/mL porcine esterase for 1 h at 37° C.

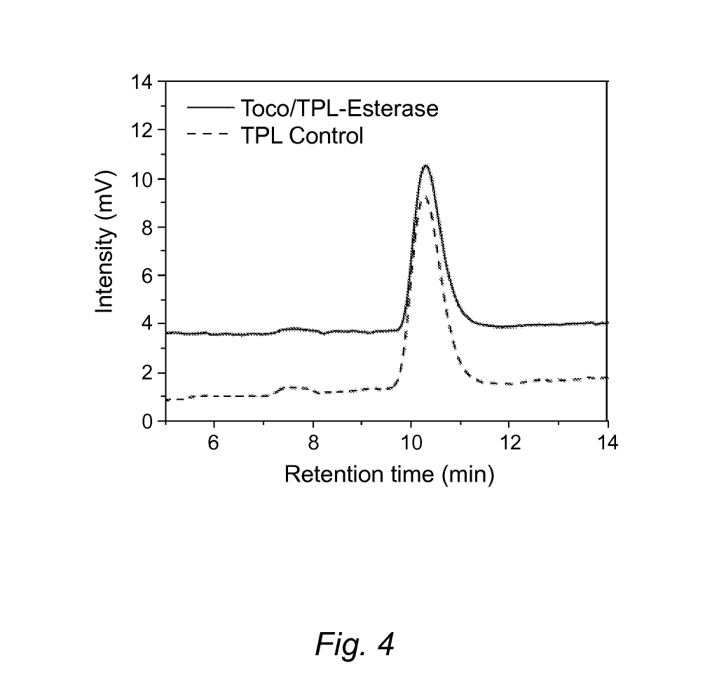

FIG. 5 depicts the inhibitory effect of triptolide (TPL) and triptolide nanoprodrug (TPL-NP) on interferon-γ-dependent expression of B7-H1 (PD-L1) on U-87 stem-like cells.

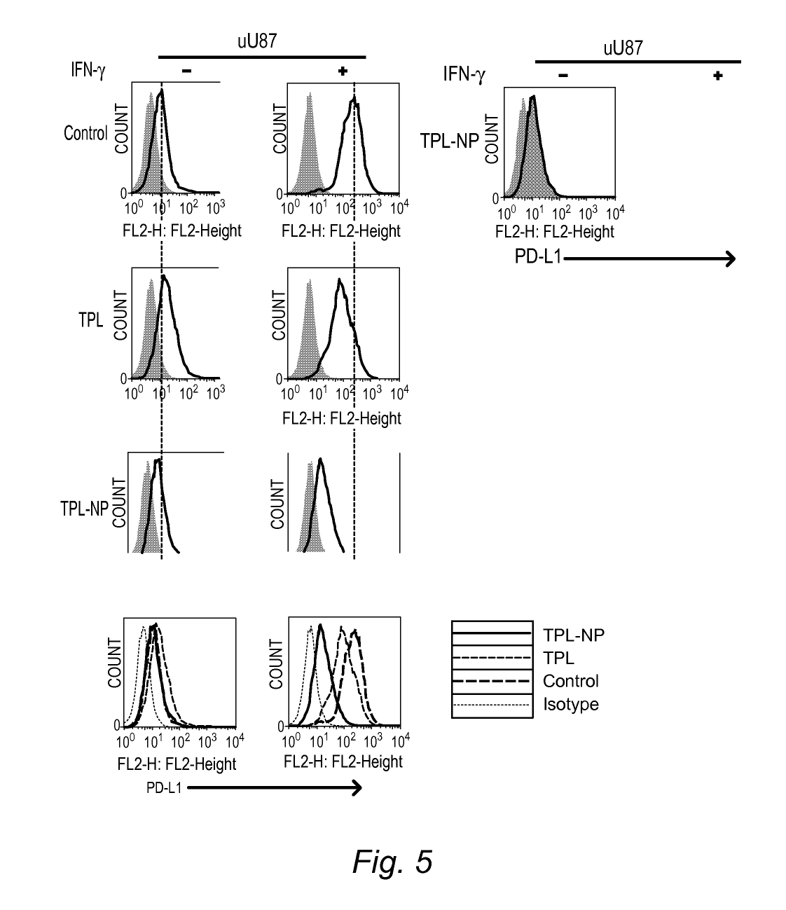

FIG. 6 depicts the inhibitory effect of triptolide (TPL) and triptolide nanoprodrug (TPL-NP) on interferon-γ-dependent expression of B7-H1 (PD-L1) on cancer stem cell (CSC-5).

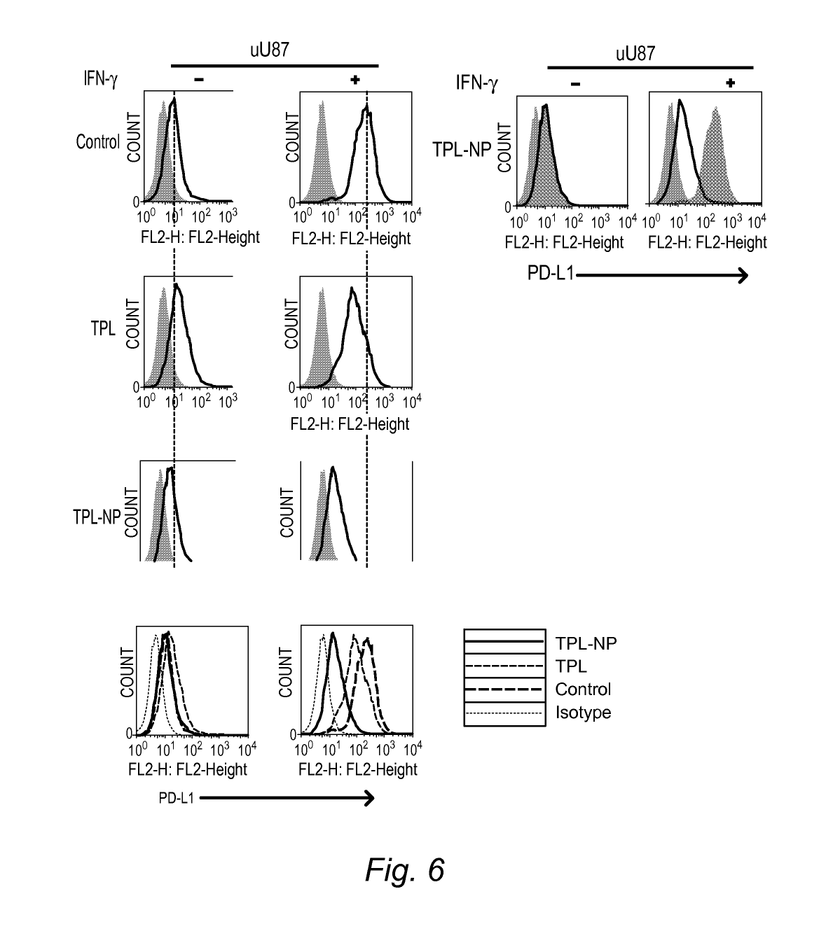

FIG. 7 depicts the inhibitory effect of triptolide (TPL) and triptolide nanoprodrug (TPL-NP) on interferon-γ-dependent expression of B7-H1 (PD-L1) on U-251 glioma cell.

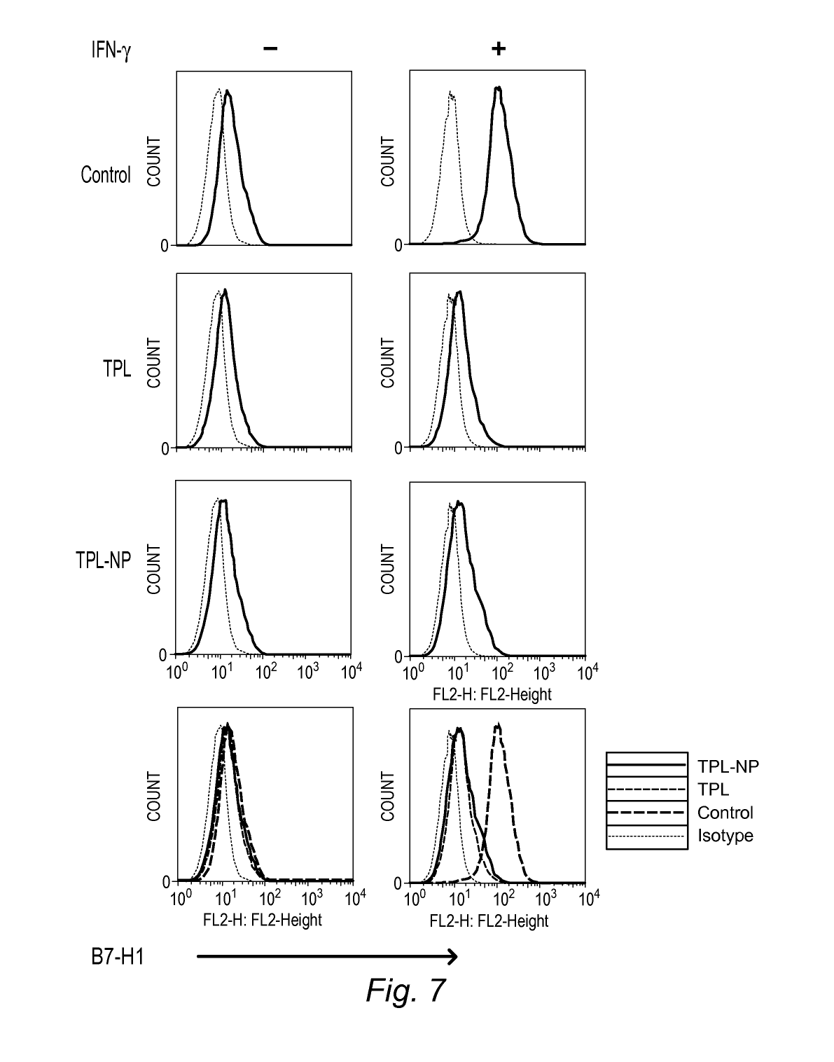

FIG. 8 depicts Inhibitory effect of triptolide (TPL) and triptolide nanoprodrug (TPL-NP) on interferon-γ-dependent expression of B7-H1 (PD-L1) on U-87 MG glioma cells.

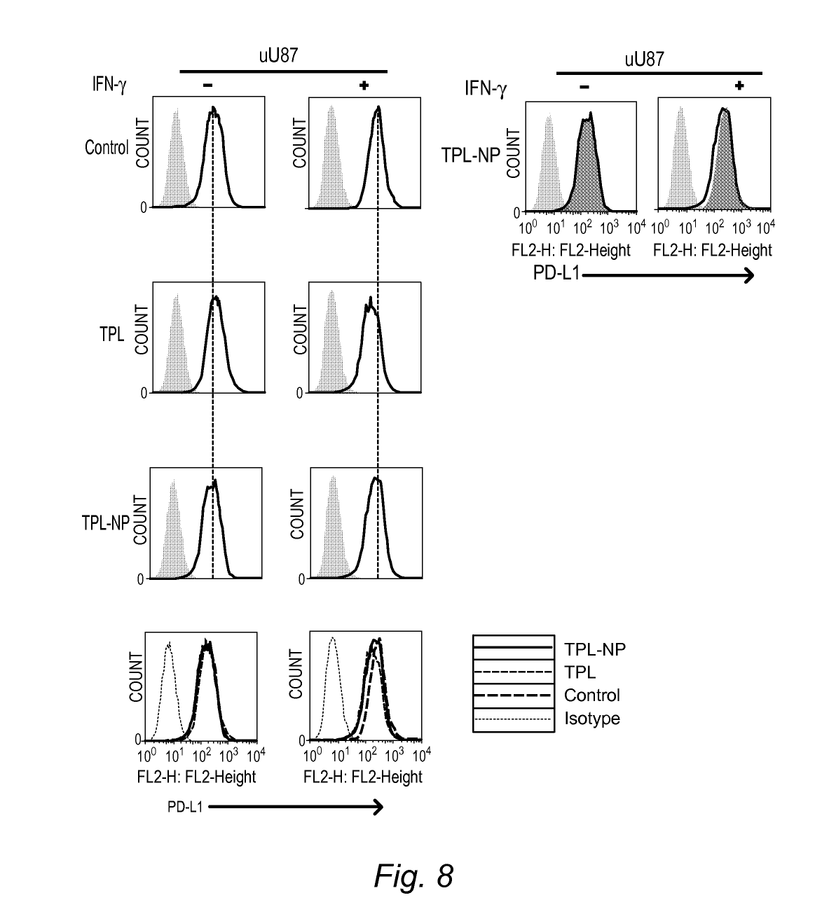

FIG. 9 depicts inhibitory effect of triptolide (TPL) and triptolide nanoprodrug (TPL-NP) on interferon-γ-dependent expression of B7-H1 (PD-L1) on MDA-MB-231 triple negative breast cancer cell.

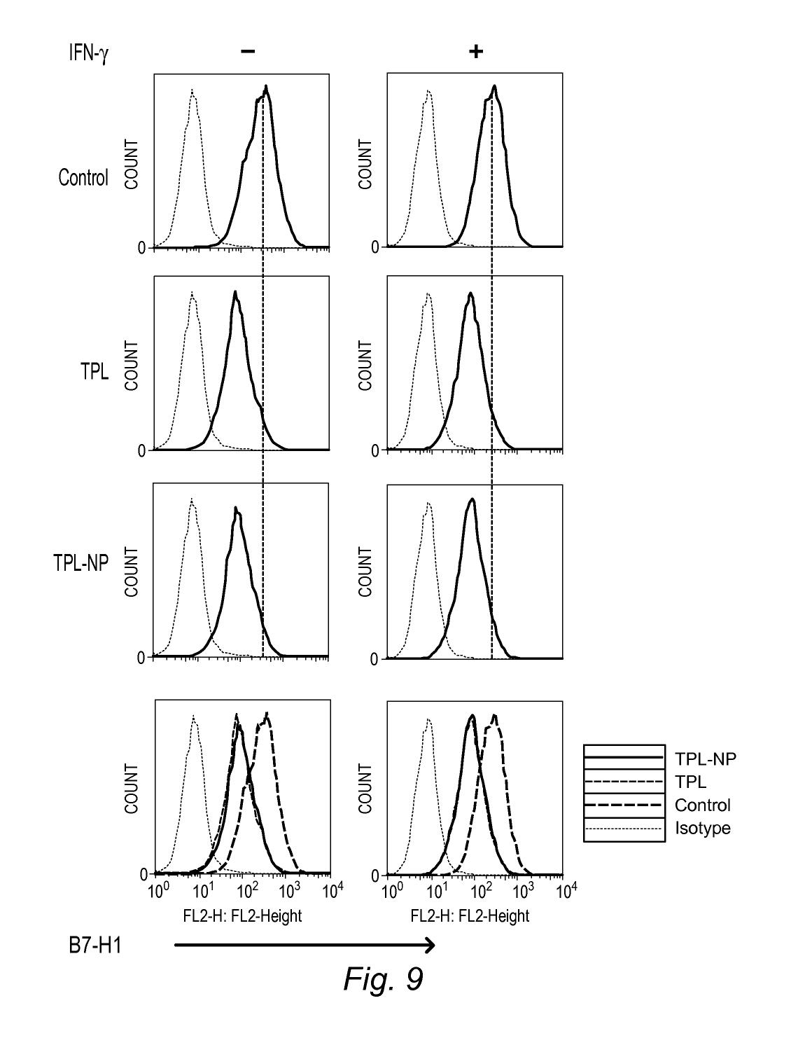

FIG. 10 depicts inhibitory effect of triptolide (TPL) and triptolide nanoprodrug (TPL-NP) on interferon-γ-dependent expression of B7-H1 (PD-L1) on MDA-MB-468 triple negative breast cancer cell.

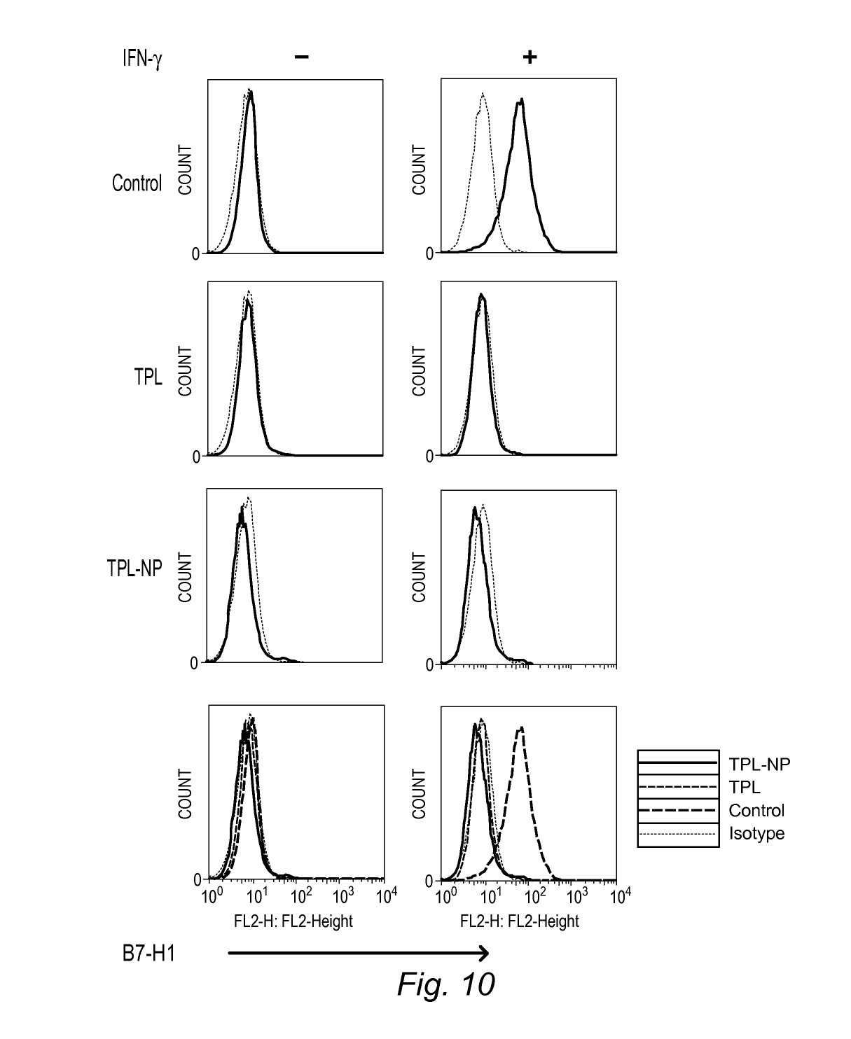

### DETAILED DESCRIPTION

Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N. Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The abbreviation “CPT” as used herein refers to camptothecin {(S)-4-ethyl-4-hydroxy-1H-pyrano-[3′,4′:6,7]indolizino[1,2-b]quinoline-3, 14(4H,12H)-dione}. The compound is commercially available from numerous sources; e.g., from Sigma Chemical Co. (St. Louis, Mo.).

“Camptothecin analogs” as used herein refer to compounds of Formula:

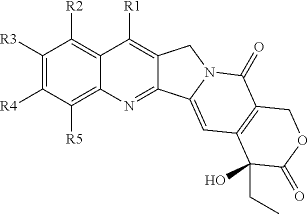

wherein R1, R2, R3, R4, and R5 is each independently selected from hydrogen or a substituent selected from an alkyl, alkoxy, aryl, cycloaliphatic, acyl, hydroxyl and aralkyl group, can be saturated or unsaturated, and can contain hetero atoms (e.g., nitrogen, oxygen, sulfur, halogens, etc).

“Cancer” and “cancerous” refer to or describe the physiological condition in mammals that is typically characterized by unregulated cell growth. Examples of cancer include, but are not limited to, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer; including, but not limited to, gliomas, glioblastomas, glioblastoma multiforme (GBM), oligodendrogliomas, primitive neuroectodermal tumors, low, mid and high grade astrocytomas, ependymomas (e.g., myxopapillary ependymoma papillary ependymoma, subependymoma, anaplastic ependymoma), oligodendrogliomas, medulloblastomas, meningiomas, pituitary carcinomas, neuroblastomas, and craniopharyngiomas.

“Beneficial results” can include, but are in no way limited to, lessening or alleviating the severity of the disease condition, preventing the disease condition from worsening, curing the disease condition and prolonging a patient's life or life expectancy. The disease conditions can relate to or can be modulated by the central nervous system.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. The terms, “patient” and “subject” are used interchangeably herein.

In some embodiments, the subject is a mammal “Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Nanosphere” as used herein refers to a particle with a size, in at least one dimension, between about 10 nm to about 1000 nm; and can also include a nanoemulsion. It will be understood by one of ordinary skill in the art that particles usually exhibit a distribution of particle sizes around the indicated “size.” Unless otherwise stated, the term “particle size” as used herein refers to the mode of a size distribution of particles, i.e., the value that occurs most frequently in the size distribution. Methods for measuring the particle size are known to a skilled artisan, e.g., by dynamic light scattering (such as photocorrelation spectroscopy, laser diffraction, low-angle laser light scattering (LALLS), and medium-angle laser light scattering (MALLS)), light obscuration methods (such as Coulter analysis method), or other techniques (such as rheology, and light or electron microscopy).

In some embodiments, the particles can be substantially spherical. What is meant by “substantially spherical” is that the ratio of the lengths of the longest to the shortest perpendicular axis of the particle cross section is less than or equal to about 1.5. Substantially spherical does not require a line of symmetry. Further, the particles can have surface texturing, such as lines or indentations or protuberances that are small in scale when compared to the overall size of the particle and still be substantially spherical. In some embodiments, the ratio of lengths between the longest and shortest axes of the particle is less than or equal to about 1.5, less than or equal to about 1.45, less than or equal to about 1.4, less than or equal to about 1.35, less than or equal to about 1.30, less than or equal to about 1.25, less than or equal to about 1.20, less than or equal to about 1.15 less than or equal to about 1.1. Without wishing to be bound by a theory, surface contact is minimized in particles that are substantially spherical, which minimizes the undesirable agglomeration of the particles upon storage. Many crystals or flakes have flat surfaces that can allow large surface contact areas where agglomeration can occur by ionic or non-ionic interactions. A sphere permits contact over a much smaller area.

The particles can be, e.g., monodispersed or polydispersed and the variation in diameter of the particles of a given dispersion can vary. In some embodiments, the particles have substantially the same particle size. Particles having a broad size distribution where there are both relatively big and small particles allow for the smaller particles to fill in the gaps between the larger particles, thereby creating new contact surfaces. A broad size distribution can result in larger spheres by creating many contact opportunities for binding agglomeration. The particles described herein are within a narrow size distribution, thereby minimizing opportunities for contact agglomeration. What is meant by a “narrow size distribution” is a particle size distribution that has a ratio of the volume diameter of the 90th percentile of the small spherical particles to the volume diameter of the 10th percentile less than or equal to 5. In some embodiments, the volume diameter of the 90th percentile of the small spherical particles to the volume diameter of the 10th percentile is less than or equal to 4.5, less than or equal to 4, less than or equal to 3.5, less than or equal to 3, less than or equal to 2.5, less than or equal to 2, less than or equal to 1.5, less than or equal to 1.45, less than or equal to 1.40, less than or equal to 1.35, less than or equal to 1.3, less than or equal to 1.25, less than or equal to 1.20, less than or equal to 1.15, or less than or equal to 1.1.

Geometric Standard Deviation (GSD) can also be used to indicate the narrow size distribution. GSD calculations involved determining the effective cutoff diameter (ECD) at the cumulative less than percentages of 15.9% and 84.1%. GSD is equal to the square root of the ratio of the ECD less than 84.17% to ECD less than 15.9%. The GSD has a narrow size distribution when GSD<2.5. In some embodiments, GSD is less than 2, less than 1.75, or less than 1.5. In one embodiment, GSD is less than 1.8.

“Nanosphere comprising TPL” and “Nanosphere prodrug comprising TPL” as used herein refer to a nanosphere comprising a TPL derivative compound. The nanosphere can further comprise a multiple α-lipoic acid-containing hydrophobic compound, α-tocopherol, an additional camptothecin derivative, an additional temozolomide derivative, an additional nonsteroidal anti-inflammatory drug (NSAID) derivative, and additional statin, an additional lipoic acid, an additional chemotherapeutic, or combinations thereof.

“Nanoprodrug” is used interchangeably with “nanosphere” throughout the application.

“Non-steroidal” as used herein distinguishes the anti-inflammatory drugs from steroids, which have a similar anti-inflammatory action.

“NSAIDs” as used herein include but are not limited to aspirin, ibuprofen, flurbiprofen, ketoprofen, fenoprofen, fenbufen, naproxen, indomethacin, diclofenac, ketorolac, tolmetin, flufenamic acid, mefenamic acid, tolfenamic acid, meclofenamic acid, niflumic acid, sulindac, and sulindac sulfide.

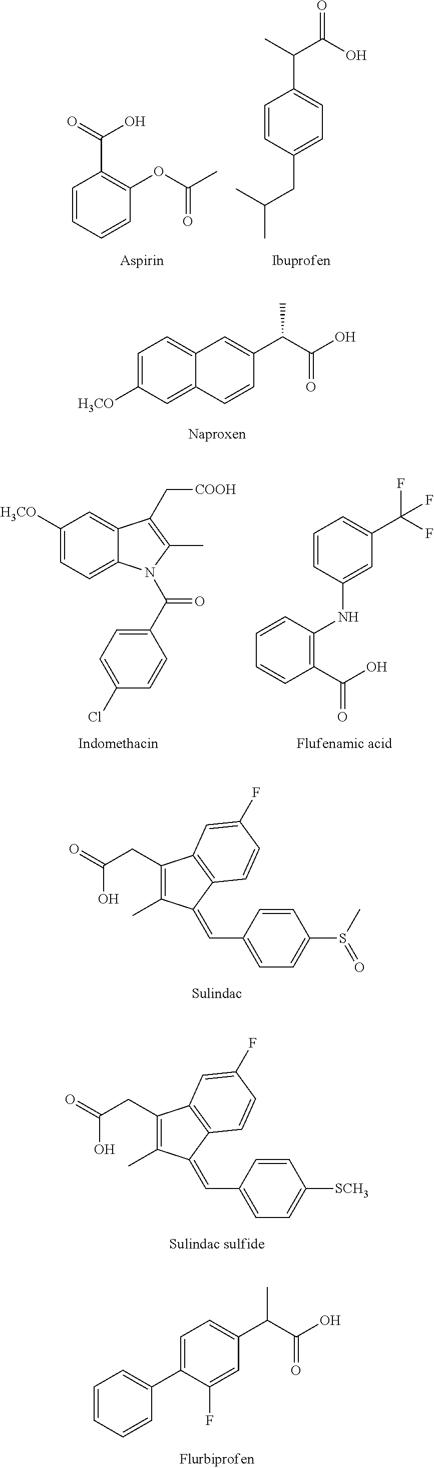

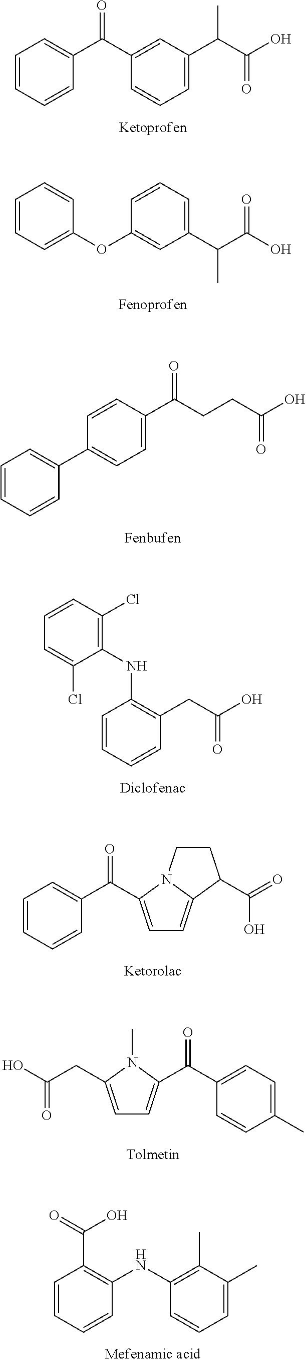

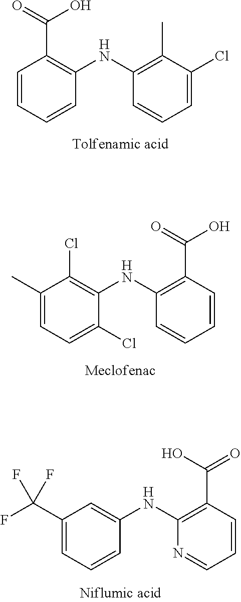 [SMILES: Cc1c(Cl)cccc1Nc1ccccc1C(=O)O ; Cc1ccc(Cl)c(Nc2ccccc2C(=O)O)c1Cl ; O=C(O)c1cccnc1Nc1cccc(C(F)(F)F)c1]

“Temozolomide carboxylic acid” as used herein refers to the temozolomide carboxylic acid derivative, which can be obtained as follows and is shown below:

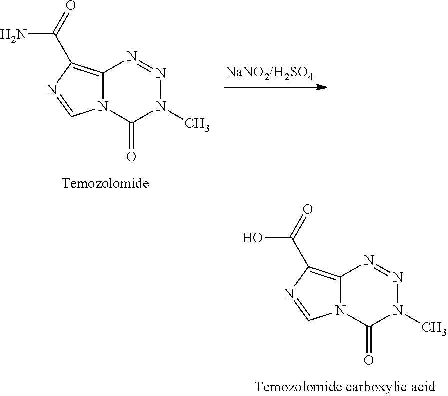

“Triptolide” or “TPL” as used herein refer to:

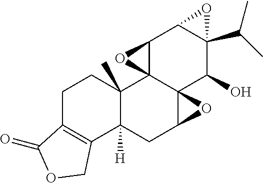 [SMILES: CC(C)[C@]12O[C@H]1[C@@H]1O[C@]13[C@]1(O[C@H]1C[C@H]1C4=C(CC[C@@]13C)C(=O)OC4)[C@@H]2O]

“Triptolide nanosphere prodrug” as used herein refers to a nanosphere containing an antioxidant, anti-inflammatory, or anticancer derivative of triptolide.

“TPL derivative” and “TPLD” and are used interchangeably as used herein refers to a compound in which at least one TPL molecule or TPL analog is coupled to an anti-inflammatory, an antioxidant or an anticancer; TPL or TPL analog can be directly conjugated to a second therapeutic agent (e.g., TPL-(second therapeutic compound(TA)) or triptolide can be conjugated to a second therapeutic agent via a spacer (e.g., TPL-spacer-(second TA)).

“TPLD nanosphere” as used herein refers to a nanosphere comprising molecules of Formula D-I and/or Formula D-II.

“Polyol” as used herein refers to a compound that contains at least two free esterifiable hydroxyl groups.

“Therapeutic agent” as used herein refers to any substance used internally or externally as a medicine for the treatment, cure, prevention, slowing down, or lessening of a disease or disorder, even if the treatment, cure, prevention, slowing down, or lessening of the disease or disorder is ultimately unsuccessful. Examples of therapeutic agents, also referred to as “drugs”, are described in well-known literature references such as the Merck Index, the Physicians Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Various forms of a therapeutic agent can be used which are capable of being released from the subject composition into adjacent tissues or fluids upon administration to a subject. Examples include steroids and esters of steroids (e.g., estrogen, progesterone, testosterone, androsterone, cholesterol, norethindrone, digoxigenin, cholic acid, deoxycholic acid, and chenodeoxycholic acid), boron-containing compounds (e.g., carborane), chemotherapeutic nucleotides, drugs (e.g., antibiotics, antivirals, antifungals), enediynes (e.g., calicheamicins, esperamicins, dynemicin, neocarzinostatin chromophore, and kedarcidin chromophore), heavy metal complexes (e.g., cisplatin), hormone antagonists (e.g., tamoxifen), non-specific (non-antibody) proteins (e.g., sugar oligomers), oligonucleotides (e.g., antisense oligonucleotides that bind to a target nucleic acid sequence (e.g., mRNA sequence)), peptides, proteins, antibodies, photodynamic agents (e.g., rhodamine 123), radionuclides (e.g., 1-131, Re-186, Re-188, Y-90, Bi-212, At-211, Sr-89, Ho-166, Sm-153, Cu-67 and Cu-64), toxins (e.g., ricin), and transcription-based pharmaceuticals.

In some embodiments, the therapeutic agent is a chemotherapeutic agent. Exemplary chemotherapeutic agents include, but are not limited to actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vinblastine, vincristine, vindesine, vinorelbine, and the like.

“Therapeutically effective amount” as used herein refers to an amount which is capable of achieving beneficial results in a patient with a condition or a disease condition in which treatment is sought. A therapeutically effective amount can be determined on an individual basis and will be based, at least in part, on consideration of the physiological characteristics of the mammal, the type of delivery system or therapeutic technique used and the time of administration relative to the progression of the disease.

“Treatment” and “treating,” as used herein refer to both therapeutic treatment and prophylactic or preventative measures, wherein the object is to prevent, slow down and/or alleviate the disease or disease condition even if the treatment is ultimately unsuccessful.

Thus, the terms “treatment” and “treating” encompass therapeutic treatments, wherein the object is to reverse, alleviate, ameliorate, inhibit, slow down or stop the progression or severity of a condition associated with a disease or disorder, e.g. cancer. The term “treating” includes reducing or alleviating at least one adverse effect or symptom of a condition, disease or disorder associated with cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “antioxidant agent” refers to a molecule that decreases, inhibits, prevents, or reduces the oxidation of an oxidizable compound. Without limitations, a compound is considered an antioxidant for purposes of this disclosure if it reduces endogenous oxygen radicals in vitro. As nonlimiting examples, antioxidants scavenge oxygen, superoxide anions, hydrogen peroxide, superoxide radicals, lipooxide radicals, hydroxyl radicals, or bind to reactive metals to prevent oxidation damage to lipids, proteins, nucleic acids, etc. Antioxidants remove free radical intermediates and inhibit other oxidation reactions by being oxidized themselves. Examples of antioxidants include, but are not limited to, hydrophilic antioxidants, lipophilic antioxidants, and mixtures thereof. Non-limiting examples of hydrophilic antioxidants include chelating agents (e.g., metal chelators) such as ethylenediaminetetraacetic acid (EDTA), citrate, ethylene glycol tetraacetic acid (EGTA), 1,2-bis(o-aminophenoxy)ethane-N,N,N′,N′-tetraacetic acid (BAPTA), diethylene triamine pentaacetic acid (DTP A), 2,3-dimercapto-1-propanesulfonic acid (DMPS), dimercaptosuccinic acid (DMSA), cc-lipoic acid, salicylaldehyde isonicotinoyl hydrazone (SIH), hexyl thioethylamine hydrochloride (HTA), desferrioxamine, salts thereof, and mixtures thereof. Additional hydrophilic antioxidants include ascorbic acid, cysteine, N-acetyl cysteine, hydrophobic derivatives of N-acetyl cysteine, glutathione, hydrophobic derivative of glutathione, dihydrolipoic acid, 2-mercaptoethane sulfonic acid, 2-mercaptobenzimidazole sulfonic acid, 6-hydroxy-2,5,7,8-tetramethylchroman-2-carboxylic acid, sodium metabisulfite, salts thereof, and mixtures thereof. Non-limiting examples of lipophilic antioxidants include vitamin E isomers such as α-, β-, γ-, and δ-tocopherols and α-, ρ-, γ-, and δ-tocotrienols; polyphenols such as 2-tert-butyl-4-methyl phenol, 2-tert-butyl-5-methyl phenol, and 2-tert-butyl-6-methyl phenol; butylated hydroxyanisole (BHA) (e.g., 2-tert-butyl-4-hydroxyanisole and 3-tert-butyl-4-hydroxyanisole); butylhydroxytoluene (BHT); quinones, e.g., tert-butylhydroquinone (TBHQ); ascorbyl palmitate; n-propyl gallate; salts thereof; and mixtures thereof.

In some embodiments, the antioxidant agent can be glutathione, hydrophobic derivative of glutathione, N-acetyl cysteine, hydrophobic, or hydrophobic derivatives of N-acetyl cysteine.

As used herein, a hydrophobic derivative of glutathione refers to a glutathione derivative comprising at least one hydrophobic group attached to one of the carboxylate groups or the amine group of glutathione. For example, the hydrophobic group can form an ester or amide with the glutathione.

As used herein, a hydrophobic derivative of N-acetyl-cysteine means a N-acetyl cysteine comprising a hydrophobic group attached to the carboxylate group of the N-acetyl cysteine. In some embodiments, the hydrophobic group can form an ester with the N-acetyl cysteine.

As used herein, the term “hydrophobic group” refers to those groups being immiscible in water. The terms “hydrophobic group” refers to any of the groups hydrogen, alkyl, alkoxy, alkoxyalkyl, aryloxy, cycloalkoxy, alkylthio, alkenyl, alkynyl, cycloalkyl, haloalkyl, alkanoyl, aroyl, aminocarbonyl, aminoalkanoyl or optionally substituted aminoalkanoyl, carbocycloalkyl or optionally substituted carbocycloalkyl, heterocyclo or optionally substituted heterocyclo, heteroaryl or optionally substituted heteroaryl, halo, aryl, aralkyl, (heterocyclo)alkyl, (heteroaryl)alkyl, alkoxycarbonyl, alkylcarbonyloxy, alkoxyalkanoyl, carboxyalkyl, amino or substituted amino, amido or substituted amido, and alkanoylamido having at least some affinity for a hydrocarbon. In some embodiments, suitable hydrophobic groups include normal or branched C₁-C₁₈-alkyl groups, arylalkyl groups and aryl groups. In some embodiments, the hydrophobic group can be a C1-18 alkyl.

As discussed elsewhere in the disclosure, the antioxidant agents can be included in the matrix of the nanoparticles or conjugated with a hydrophilic spacer, a hydrophobic spacer, an amphiphilic spacer, or an amphiphilic polymer in the nanosphere. Without wishing to be bound by a theory, an antioxidant agent conjugated with a hydrophilic spacer, a hydrophobic spacer, an amphiphilic spacer, or an amphiphilic polymer in the nanosphere can be present on the surface of the nanosphere. Accordingly, unmodified glutathione and N-acetyl-cysteine can be present conjugated on the surface of the nanosphere by conjugating with a hydrophilic spacer, a hydrophobic spacer, an amphiphilic spacer, or an amphiphilic polymer in the nanosphere. Hydrophobically modified glutathione and N-acetyl-cysteine derivatives can be included in the matrix of the nanoparticles

“Antioxidant and TPLD nanosphere” and “TPLD nanosphere and Antioxidant nanosphere” as used herein refer to a nanosphere comprising molecules of Formula D-I and/or Formula D-II.

“Antioxidant nanosphere” as used herein refers to a nanosphere comprising molecules of Formula A-IV and/or A-V.

“TPL/Antioxidant nanosphere combination” and “Antioxidant/TPLD nanosphere combination” as used herein refer to a nanosphere comprising molecules selected from Formula D-I or Formula D-II and molecules selected from Formula A-IV or A-V.

“TPLD nanosphere/Antioxidant nanosphere composition” and “Antioxidant nanosphere/TPLD nanosphere composition” as used herein refer to a composition comprising Antioxidant nanospheres in combination with TPLD nanospheres or Antioxidant and TPLD nanospheres.

As used herein, the term “aliphatic” means a moiety characterized by a straight or branched chain arrangement of constituent carbon atoms and can be saturated or partially unsaturated with one or more (e.g., one, two, three, four, five or more) double or triple bonds.

As used herein, the term “alicyclic” means a moiety comprising a nonaromatic ring structure. Alicyclic moieties can be saturated or partially unsaturated with one or more double or triple bonds. Alicyclic moieties can also optionally comprise heteroatoms such as nitrogen, oxygen and sulfur. The nitrogen atoms can be optionally quaternerized or oxidized and the sulfur atoms can be optionally oxidized. Examples of alicyclic moieties include, but are not limited to moieties with C₃-C₈ rings such as cyclopropyl, cyclohexane, cyclopentane, cyclopentene, cyclopentadiene, cyclohexane, cyclohexene, cyclohexadiene, cycloheptane, cycloheptene, cycloheptadiene, cyclooctane, cyclooctene, and cyclooctadiene.

As used herein, the term, “aromatic” means a moiety wherein the constituent atoms make up an unsaturated ring system, all atoms in the ring system are sp² hybridized and the total number of pi electrons is equal to 4n+2. An aromatic ring canbe such that the ring atoms are only carbon atoms (e.g., aryl) or can include carbon and non-carbon atoms (e.g., heteroaryl).

As used herein, the term “alkyl” means a straight or branched, saturated aliphatic radical having a chain of carbon atoms. Cₓ alkyl and Cₓ-C_{y}alkyl are typically used where X and Y indicate the number of carbon atoms in the chain. For example, C₁-C₆alkyl includes alkyls that have a chain of between 1 and 6 carbons (e.g., methyl, ethyl, propyl, isopropyl, butyl, sec-butyl, isobutyl, tert-butyl, pentyl, neopentyl, hexyl, and the like). Alkyl represented along with another radical (e.g., as in arylalkyl) means a straight or branched, saturated alkyl divalent radical having the number of atoms indicated or when no atoms are indicated means a bond, e.g., (C₆-C₁₀)aryl(C₀-C₃)alkyl includes phenyl, benzyl, phenethyl, 1-phenylethyl 3-phenylpropyl, and the like. Backbone of the alkyl can be optionally inserted with one or more heteroatoms, such as N, O, or S.

In preferred embodiments, a straight chain or branched chain alkyl has 30 or fewer carbon atoms in its backbone (e.g., C1-C30 for straight chains, C3-C30 for branched chains), and more preferably 20 or fewer. Likewise, preferred cycloalkyls have from 3-10 carbon atoms in their ring structure, and more preferably have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

In some embodiments, alkyl is C1-2 alkyl, C1-3 alkyl, C1-4 alkyl, C1-6 alkyl, C1-8 alkyl, C1-10 alkyl or C1-12 alkyl. In some embodiments, the branched and unbranched alkyl, is C2-3 alkyl, C2-4 alkyl, C2-6 alkyl, C2-8 alkyl, C2-10 alkyl or C2-12 alkyl.

Substituents of a substituted alkyl can include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF3, —CN and the like.

As used herein, the term “alkenyl” refers to unsaturated straight-chain, branched-chain or cyclic hydrocarbon radicals having at least one carbon-carbon double bond. Cₓ alkenyl and Cₓ-C_{y}alkenyl are typically used where X and Y indicate the number of carbon atoms in the chain. For example, C₂-C₆alkenyl includes alkenyls that have a chain of between 1 and 6 carbons and at least one double bond, e.g., vinyl, allyl, 1-propenyl, isopropenyl, 1-butenyl, 2-butenyl, 3-butenyl, 2-methylallyl, 1-hexenyl, 2-hexenyl, 3-hexenyl, and the like). Alkenyl represented along with another radical (e.g., as in arylalkenyl) means a straight or branched, alkenyl divalent radical having the number of atoms indicated. Backbone of the alkenyl can be optionally inserted with one or more heteroatoms, such as N, O, or S.

In some embodiments, the branched and unbranched alkenyl, is C2-3 alkenyl, C2-4 alkenyl, C2-6 alkenyl, C2-8 alkenyl, C2-10 alkenyl or C2-12 alkenyl. As used herein, the term “alkynyl” refers to unsaturated hydrocarbon radicals having at least one carbon-carbon triple bond. Cₓ alkynyl and Cₓ-C_{y}alkynyl are typically used where X and Y indicate the number of carbon atoms in the chain. For example, C₂-C₆alkynyl includes alkynls that have a chain of between 1 and 6 carbons and at least one triple bond, e.g., ethynyl, 1-propynyl, 2-propynyl, 1-butynyl, isopentynyl, 1,3-hexa-diyn-yl, n-hexynyl, 3-pentynyl, 1-hexen-3-ynyl and the like. Alkynyl represented along with another radical (e.g., as in arylalkynyl) means a straight or branched, alkynyl divalent radical having the number of atoms indicated. Backbone of the alkynyl can be optionally inserted with one or more heteroatoms, such as N, O, or S.

In some embodiments, the branched and unbranched alkynyl, is C2-3 alkynyl, C2-4 alkynyl, C2-6 alkynyl, C2-8 alkynyl, C2-10 alkynyl or C2-12alkynyl.

The terms “alkylene,” “alkenylene,” and “alkynylene” refer to divalent alkyl, alkelyne, and alkynylene” radicals. Prefixes Cₓ and Cₓ-C_{y} are typically used where X and Y indicate the number of carbon atoms in the chain. For example, C₁-C₆alkylene includes methylene, (—CH₂—), ethylene (—CH₂CH₂—), trimethylene (—CH₂CH₂CH₂—), tetramethylene (—CH₂CH₂CH₂CH₂—), 2-methyltetramethylene (—CH₂CH(CH₃)CH₂CH₂—), pentamethylene (—CH₂CH₂CH₂CH₂CH₂—) and the like).

As used herein, the term “alkylidene” means a straight or branched unsaturated, aliphatic, divalent radical having a general formula ═CRₐR_{b}. Cₓ alkylidene and Cₓ-C_{y}alkylidene are typically used where X and Y indicate the number of carbon atoms in the chain. For example, C₂-C₆alkylidene includes methylidene (═CH₂), ethylidene (═CHCH₃), isopropylidene (═C(CH₃)₂), propylidene (═CHCH₂CH₃), allylidene (═CH—CH═CH₂), and the like).

The term “aralkyl” or “arylalkyl” group comprises an aryl group covalently attached to an alkyl group, either of which independently is optionally substituted. Preferably, the aralkyl group is C₆₋₁₀ aryl(C₆₋₁₀)alkyl, including, without limitation, benzyl, phenethyl, and naphthylmethyl.

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application. For example, halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl, 2,2,2-trifluoroethyl, perfluoroethyl, 2,2,2-trifluoro-1,1-dichloroethyl, and the like).

The term “aryl” or “cyclic aromatic” refers to monocyclic, bicyclic, or tricyclic fused aromatic ring system. Cₓ aryl and Cₓ-C_{y}aryl are typically used where X and Y indicate the number of carbon atoms in the ring system. Exemplary aryl groups include, but are not limited to, benzyl, phenyl, naphthyl, anthracenyl, azulenyl, fluorenyl, indanyl, indenyl, naphthyl, phenyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

In some embodiments, the cyclic aromatic is C4, C5, C6, C7 or C8 cyclic aromatic. In some embodiments, the cyclic aromatic is C8-12 cyclic aromatic.

The term “heteroaryl” or “aromatic heterocyclic” refers to an aromatic 5-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively. Cₓ heteroaryl and Cₓ-C_{y}heteroaryl are typically used where X and Y indicate the number of carbon atoms in the ring system. Heteroaryls include, but are not limited to, those derived from benzo[b]furan, benzo[b]thiophene, benzimidazole, imidazo[4,5-c]pyridine, quinazoline, thieno[2,3-c]pyridine, thieno[3,2-b]pyridine, thieno[2, 3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3c]pyridine, pyrrolo[3,2-c]pyridine, pyrrolo[3,2-b]pyridine, pyrrolo[2,3-d]pyrimidine, pyrrolo[3,2-d]pyrimidine, pyrrolo[2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole, 2(1H)-pyridinone, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Some exemplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, 2-amino-4-oxo-3,4-dihydropteridin-6-yl, tetrahydroisoquinolinyl, and the like. In some embodiments, 1, 2, 3, or 4 hydrogen atoms of each ring can be substituted by a substituent.

In some embodiments, the aromatic heterocyclic is C4, C5, C6, C7 or C8 aromatic heterocyclic. In some embodiments, the aromatic heterocyclic is C8-12 aromatic heterocyclic

The term “cyclyl” or “cycloalkyl” or “cyclic aliphatic” refers to saturated and partially unsaturated cyclic hydrocarbon groups having 3 to 12 carbons, for example, 3 to 8 carbons, and, for example, 3 to 6 carbons. Cₓcyclyl and Cₓ-C_{y}cylcyl are typically used where X and Y indicate the number of carbon atoms in the ring system. The cycloalkyl group additionally can be optionally substituted, e.g., with 1, 2, 3, or 4 substituents. C₃-C₁₀cyclyl includes cyclopropyl, cyclobutyl, cyclopentyl, cyclohexyl, cyclohexenyl, 2,5-cyclohexadienyl, cycloheptyl, cyclooctyl, bicyclo[2.2.2]octyl, adamantan-1-yl, decahydronaphthyl, oxocyclohexyl, dioxocyclohexyl, thiocyclohexyl, 2-oxobicyclo[2.2.1]hept-1-yl, and the like.

In some embodiments, the cyclic aliphatic is C3, C4, C5, C6, C7, or C8 cyclic aliphatic. In some embodiments, the cyclic aliphatic is C8-12 cyclic aliphatic.

Aryl and heteroaryls can be optionally substituted with one or more substituents at one or more positions, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, —CN, or the like.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓheterocyclyl and Cₓ-C_{y}heterocyclyl are typically used where X and Y indicate the number of carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyland the like.

In some embodiments, the heterocyclic is C4, C5, C6, C7 or C8 heterocyclic. In some embodiments, the heterocyclic is C8-12 heterocyclic.

The terms “bicyclic” and “tricyclic” refers to fused, bridged, or joined by a single bond polycyclic ring assemblies.

The term “cyclylalkylene” means a divalent aryl, heteroaryl, cyclyl, or heterocyclyl.

As used herein, the term “fused ring” refers to a ring that is bonded to another ring to form a compound having a bicyclic structure when the ring atoms that are common to both rings are directly bound to each other. Non-exclusive examples of common fused rings include decalin, naphthalene, anthracene, phenanthrene, indole, furan, benzofuran, quinoline, and the like. Compounds having fused ring systems can be saturated, partially saturated, cyclyl, heterocyclyl, aromatics, heteroaromatics, and the like.

As used herein, the term “carbonyl” means the radical —C(O)—. It is noted that the carbonyl radical can be further substituted with a variety of substituents to form different carbonyl groups including acids, acid halides, amides, esters, ketones, and the like.

The term “carboxy” means the radical —C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like.

The term “cyano” means the radical —CN.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}—, —N⁺(O⁻)═, —O—, —S— or —S(O)₂—, —OS(O)₂—, and —SS—, wherein R^{N} is H or a further substituent.

In some embodiments, the heteroatom is N. In some embodiments, the heteroatom is O.

In some embodiments, the heteroatom is S.

The term “hydroxy” means the radical —OH.

The term “imine derivative” means a derivative comprising the moiety —C(NR)—, wherein R comprises a hydrogen or carbon atom alpha to the nitrogen.

The term “nitro” means the radical —NO₂.

An “oxaaliphatic,” “oxaalicyclic”, or “oxaaromatic” mean an aliphatic, alicyclic, or aromatic, as defined herein, except where one or more oxygen atoms (—O—) are positioned between carbon atoms of the aliphatic, alicyclic, or aromatic respectively.

An “oxoaliphatic,” “oxoalicyclic”, or “oxoaromatic” means an aliphatic, alicyclic, or aromatic, as defined herein, substituted with a carbonyl group. The carbonyl group can be an aldehyde, ketone, ester, amide, acid, or acid halide. As used herein, the term, “aromatic” means a moiety wherein the constituent atoms make up an unsaturated ring system, all atoms in the ring system are sp² hybridized and the total number of pi electrons is equal to 4n+2. An aromatic ring canbe such that the ring atoms are only carbon atoms (e.g., aryl) or can include carbon and non-carbon atoms (e.g., heteroaryl).

As used herein, the term “substituted” refers to independent replacement of one or more (typically 1, 2, 3, 4, or 5) of the hydrogen atoms on the substituted moiety with substituents independently selected from the group of substituents listed below in the definition for “substituents” or otherwise specified. In general, a non-hydrogen substituent can be any substituent that can be bound to an atom of the given moiety that is specified to be substituted. Examples of substituents include, but are not limited to, acyl, acylamino, acyloxy, aldehyde, alicyclic, aliphatic, alkanesulfonamido, alkanesulfonyl, alkaryl, alkenyl, alkoxy, alkoxycarbonyl, alkyl, alkylamino, alkylcarbanoyl, alkylene, alkylidene, alkylthios, alkynyl, amide, amido, amino, amino, aminoalkyl, aralkyl, aralkylsulfonamido, arenesulfonamido, arenesulfonyl, aromatic, aryl, arylamino, arylcarbanoyl, aryloxy, azido, carbamoyl, carbonyl, carbonyls (including ketones, carboxy, carboxylates, CF₃, cyano (CN), cycloalkyl, cycloalkylene, ester, ether, haloalkyl, halogen, halogen, heteroaryl, heterocyclyl, hydroxy, hydroxy, hydroxyalkyl, imino, iminoketone, ketone, mercapto, nitro, oxaalkyl, oxo, oxoalkyl, phosphoryl (including phosphonate and phosphinate), silyl groups, sulfonamido, sulfonyl (including sulfate, sulfamoyl and sulfonate), thiols, and ureido moieties, each of which can optionally also be substituted or unsubstituted. In some cases, two substituents, together with the carbon(s) to which they are attached to, can form a ring.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An“ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The term “aralkyl”, as used herein, refers to an alkyl group substituted with an aryl group (e.g., an aromatic or heteroaromatic group).

The term “alkylthio” refers to an alkyl group, as defined above, having a sulfur radical attached thereto. In preferred embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids, sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

As used herein, the term “amino” means —NH₂. The term “alkylamino” means a nitrogen moiety having at least one straight or branched unsaturated aliphatic, cyclyl, or heterocyclyl radicals attached to the nitrogen. For example, representative amino groups include —NH₂, —NHCH₃, —N(CH₃)₂, —NH(C₁-C₁₀alkyl), —N(C₁-C₁₀alkyl)₂, and the like. The term “alkylamino” includes “alkenylamino,” “alkynylamino,” “cyclylamino,” and “heterocyclylamino.” The term “arylamino” means a nitrogen moiety having at least one aryl radical attached to the nitrogen. For example —NHaryl, and N(aryl)₂. The term “heteroarylamino” means a nitrogen moiety having at least one heteroaryl radical attached to the nitrogen. For example —NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

It is noted in regard to all of the definitions provided herein that the definitions should be interpreted as being open ended in the sense that further substituents beyond those specified can be included. Hence, a C₁ alkyl indicates that there is one carbon atom but does not indicate what are the substituents on the carbon atom. Hence, a C₁ alkyl comprises methyl (i.e., —CH3) as well as CRₐR_{b}R_{c} where Rₐ, R_{b}, and R_{c} can each independently be hydrogen or any other substituent where the atom alpha to the carbon is a heteroatom or cyano. Hence, CF₃, CH₂OH and CH₂CN are all C₁ alkyls.

The term “derivative” as used herein refers to a chemical substance related structurally to another, i.e., an “original” substance, which can be referred to as a “parent” compound. A “derivative” can be made from the structurally-related parent compound in one or more steps. In some embodiments, the general physical and chemical properties of a derivative can be similar to or different from the parent compound.

Drug Delivery system (DDS) is used to deliver cytotoxic chemotherapeutic drugs and can effectively target tumor tissue. Thus, healthy organs, tissues or cells can be protected from the toxic activity of the drugs. The prodrug strategy offers a similar advantage over conventional treatment. Drugs will be released from the prodrugs selective in the site of diseased organs or tissues and a significant reduction of adverse side effects can be achieved. The inventors have previously demonstrated that nanoprodrugs are highly selectively and can accumulate in the tumor tissues. (see U.S. patent application Ser. No. 12/995,125 and PCT/US2012/048703)

The present invention provides novel triptolide derivatives. In some embodiments, the triptolide derivatives are hydrophobic triptolide prodrugs which can be transformed into a nanoprodrug by spontaneous hydrophobic assembly. Triptolide has an anti-immunosuppressive effect, and enhance anticancer immune response of the body. The triptolide derivatives (TPLD) of the invention combines targeted antioxidant, anti-inflammatory, and anticancer therapy with cancer immunotherapy.

The present invention provides for antioxidant, anti-inflammatory, and anticancer derivatives of triptolide. These derivatives are useful for treating various types of cancer. In addition, the present invention provides antioxidant, anti-inflammatory, and antineoplastic nanospheres comprising the derivatives of triptolide and methods of preparing the antioxidant, anti-inflammatory, and antineoplastic nanospheres. These nanospheres can operate as prodrugs.

In one embodiment, the triptolide nanosphere prodrugs are capable of releasing triptolide for a prolonged period of time. In another embodiment, the triptolide nanosphere prodrugs are capable of serving as a vehicle for the delivery of additional pharmaceuticals.

In some embodiments, TPLD is a TPL-chemotherapeutic agent. In some embodiments, TPLD is TPL-NSAID. In some embodiments, TPLD is TPL-statin. In some embodiments, TPLD is TPL-alpha lipoic acid.

In some embodiments, TPLD comprises TPL directly conjugated to a second therapeutic agent (TPL-(second therapeutic compound (TA)). In some embodiments, TPLD comprises TPL conjugated to a second therapeutic agent via a spacer (TPL-spacer-(second TA).

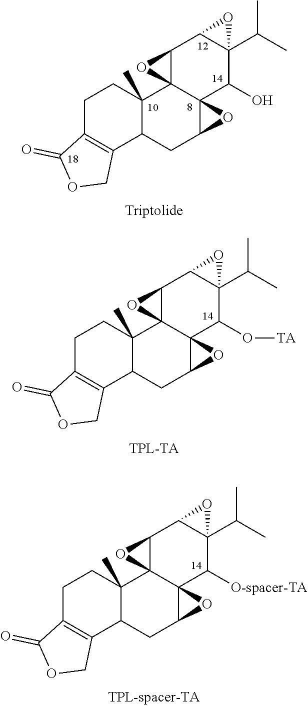

In some embodiments, TPLD is triptolide-α-lipoic acid (“TPL-ALA”).

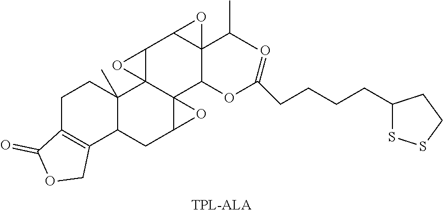 [SMILES: CC(C)C12OC1C1OC13C1(C)CCC4=C(COC4=O)C1CC1OC13C2OC(=O)CCCCC1CCSS1]

In some embodiments, TPLD is triptolide-temozolomide.

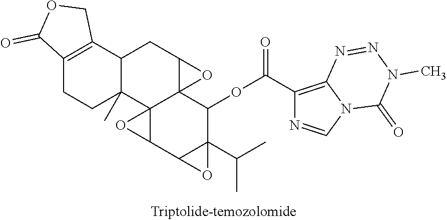 [SMILES: CC(C)C12OC1C1OC13C1(C)CCC4=C(COC4=O)C1CC1OC13C2OC(=O)c1ncn2c(=O)n(C)nnc12]

In some embodiments, TPLD is Triptolide-camptothecin.

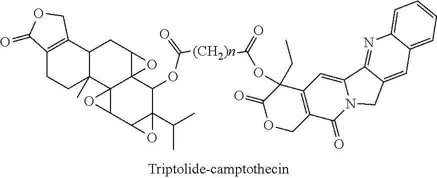

wherein n is an integer 2-12.

In some embodiments, TPLD is triptolide-paclitaxel.

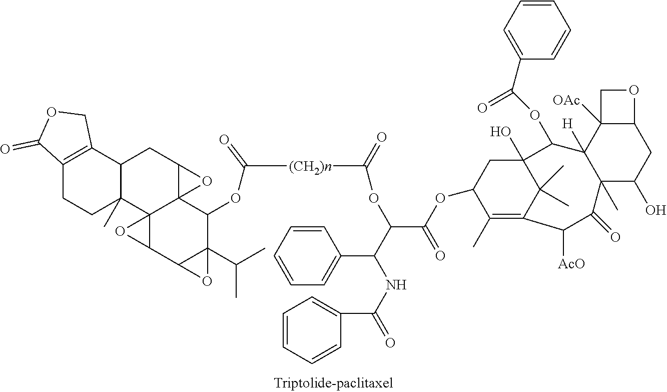

wherein n is an integer 2-12.

In some embodiments, TPLD is Triptolide-NSAID. In some embodiments, triptolide-NSAID is

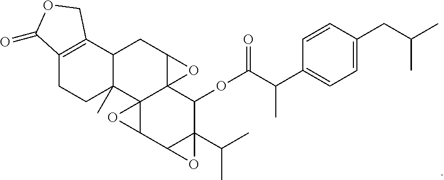 [SMILES: CC(C)Cc1ccc(C(C)C(=O)OC2C3(C(C)C)OC3C3OC34C3(C)CCC5=C(COC5=O)C3CC3OC324)cc1]

In some embodiments, triptolide-NSAID is

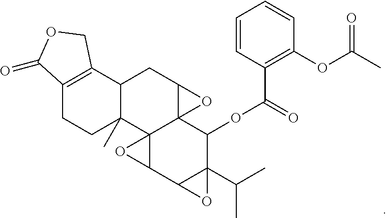 [SMILES: CC(=O)Oc1ccccc1C(=O)OC1C2(C(C)C)OC2C2OC23C2(C)CCC4=C(COC4=O)C2CC2OC213]

In some embodiments, TPLD is triptolide-statin. In some embodiments, triptolide-statin is

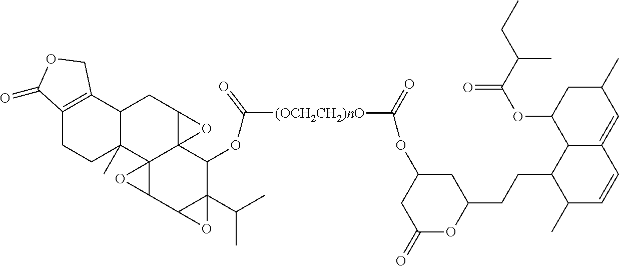

wherein n is an integer 2-12.

In one embodiment, an antioxidant, anti-inflammatory, and anticancer derivative of triptolide is prepared by the conjugation of a triptolide and an antioxidant, anti-inflammatory, or anticancer molecules and is represented by Formula D-I or Formula D-II:

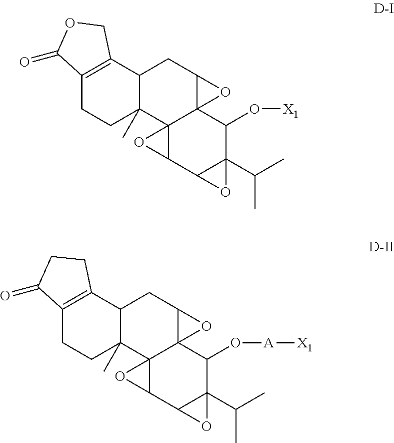

wherein:
X1 is selected from the group of antioxidant, anti-inflammatory, or anticancer molecules consisting of α-lipoic acid, temozolomide carboxylic acid, ibuprofen, ketoprofen, fenoprofen, fenprofen, fluriprofen, tolmetin, ketorolac, indomethacin, aspirin, flufenamic acid, naproxen, mefenamic acid, sulindac, diclofenac, atorvastatin, fluvastatin, rosuvastatin, lovastatin, pitavastatin, simvastatin, and mevastatin, and
A is selected from the group consisting of branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic groups.

Exemplary branched or unbranched alkyl for A in molecules of Formula D-I or Formula D-II include, but are not limited to, C1-2 alkyl, C1-3 alkyl, C1-4 alkyl, C1-6 alkyl, C1-8 alkyl, C1-10 alkyl or C1-12 alkyl. In some embodiments, A is a branched or unbranched alkyl comprising one, two, three, four, five, six, seven, eight, nine, ten, eleven, twelve or more carbons.

Exemplary branched or unbranched alkenyl for A in molecules of Formula D-I or Formula D-II include, but are not limited to, C2-3 alkenyl, C2-4 alkenyl, C2-6 alkenyl, C2-8 alkenyl, C2-10 alkenyl or C2-12 alkenyl. In some embodiments, A is a branched or unbranched alkenyl comprising two, three, four, five, six, seven, eight, nine, ten, eleven, twelve or more carbons.

Exemplary branched or unbranched alkynyl for A in molecules of Formula D-I or Formula D-II include, but are not limited to, C2-3 alkynyl, C2-4 alkynyl, C2-6 alkynyl, C2-8 alkynyl, C2-10 alkynyl or C2-12 alkynyl. In some embodiments, A is a branched or unbranched alkynyl comprising two, three, four, five, six, seven, eight, nine, ten, eleven, twelve or more carbons.

When the A is an heteroatom-containing branched or unbranched alkyl, heteroatom-containing branched or unbranched alkenyl, or heteroatom-containing branched or unbranched alkynyl, each heteroatom can be selected independently from N, O, and S. Accordingly, in some embodiments, the heteroatom is N. In some embodiments, heteroatom is O. In some embodiments, the heteroatom is S.

Exemplary cyclic aliphatic for A in molecules of Formula D-I or Formula D-II include, but are not limited to C3-12 cyclic aliphatic. For example, the cyclic aliphatic can be a C3, C4, C5, C6, C7, or C8 cyclic aliphatic. In some embodiments, the cyclic aliphatic is C8-12 cyclic aliphatic.

Exemplary cyclic aromatics for A in molecules of Formula D-I or Formula D-II include, but are not limited to C4-12 cyclic aromatics. For example, the cyclic aromatic can be a C4, C5, C6, C7, or C8 cyclic aromatic. In some embodiments, the cyclic aromatic is C8-12 cyclic aromatic.

Exemplary heterocyclic for A in molecules of Formula D-I or Formula D-II include, but are not limited to C4-12 heterocyclic. For example, the cyclic aromatic can be a C4, C5, C6, C7, or C8 heterocyclic. In some embodiments, the heterocyclic is C8-12 cyclic heterocyclic.

In various embodiments, A in molecules of Formula D-I or Formula D-II can be a polyol or a moiety that is formed by esterification of at least two free esterifiable hydroxyl groups on a polyol. In some embodiments, the polyol can be HO(CH₂CH₂O)ₙH, wherein n on the polyol can be an integer between 1 and 6. In some embodiments, the polyol can be HO(CH₂)—OH, wherein n on the polyol can be an integer between 3 and 16.

In other embodiments, A in molecules of Formula D-I or Formula D-II can be or formed from esterification of a polyol selected from group consisting of an ethylene glycol, diethylene glycol, triethylene glycol, tetraethylene glycol, pentaethylene glycol, hexaethylene glycol, 1,3-propanediol, and 1,4-butanediol. In some embodiments, the polyol can be selected from the commercial available polyols as shown in Table 1.

Examples of particularly useful antioxidant, anti-inflammatory derivatives of triptolide of this embodiment are:

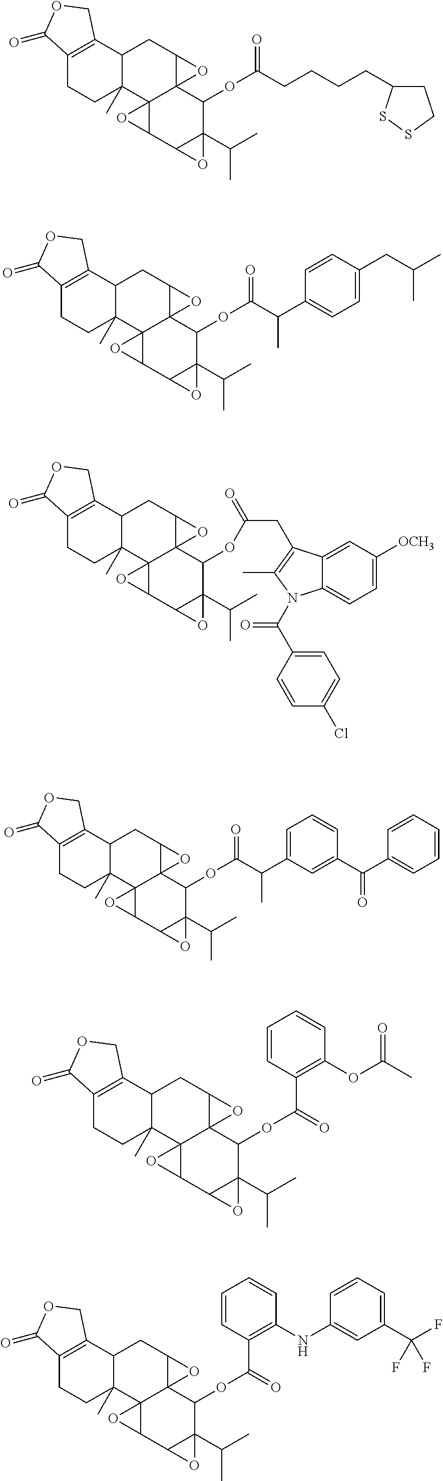 [SMILES: CC(C)C12OC1C1OC13C1(C)CCC4=C(COC4=O)C1CC1OC13C2OC(=O)CCCCC1CCSS1 ; CC(C)Cc1ccc(C(C)C(=O)OC2C3(C(C)C)OC3C3OC34C3(C)CCC5=C(COC5=O)C3CC3OC324)cc1 ; COc1ccc2c(c1)c(CC(=O)OC1C3(C(C)C)OC3C3OC34C3(C)CCC5=C(COC5=O)C3CC3OC314)c(C)n2C(=O)c1ccc(Cl)cc1 ; CC(C(=O)OC1C2(C(C)C)OC2C2OC23C2(C)CCC4=C(COC4=O)C2CC2OC213)c1cccc(C(=O)c2ccccc2)c1 ; CC(=O)Oc1ccccc1C(=O)OC1C2(C(C)C)OC2C2OC23C2(C)CCC4=C(COC4=O)C2CC2OC213 ; CC(C)C12OC1C1OC13C1(C)CCC4=C(COC4=O)C1CC1OC13C2OC(=O)c1ccccc1Nc1cccc(C(F)(F)F)c1]

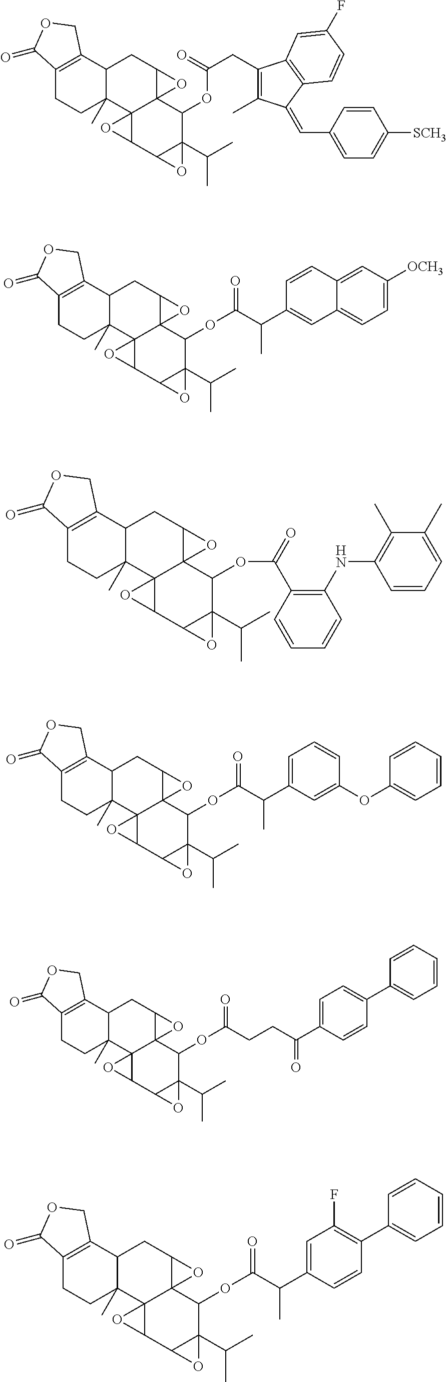 [SMILES: CSc1ccc(/C=C2/C(C)=C(CC(=O)OC3C4(C(C)C)OC4C4OC45C4(C)CCC6=C(COC6=O)C4CC4OC435)c3cc(F)ccc32)cc1 ; COc1ccc2cc(C(C)C(=O)OC3C4(C(C)C)OC4C4OC45C4(C)CCC6=C(COC6=O)C4CC4OC435)ccc2c1 ; Cc1cccc(Nc2ccccc2C(=O)OC2C3(C(C)C)OC3C3OC34C3(C)CCC5=C(COC5=O)C3CC3OC324)c1C ; CC(C(=O)OC1C2(C(C)C)OC2C2OC23C2(C)CCC4=C(COC4=O)C2CC2OC213)c1cccc(Oc2ccccc2)c1 ; CC(C)C12OC1C1OC13C1(C)CCC4=C(COC4=O)C1CC1OC13C2OC(=O)CCC(=O)c1ccc(-c2ccccc2)cc1 ; CC(C(=O)OC1C2(C(C)C)OC2C2OC23C2(C)CCC4=C(COC4=O)C2CC2OC213)c1ccc(-c2ccccc2)c(F)c1]

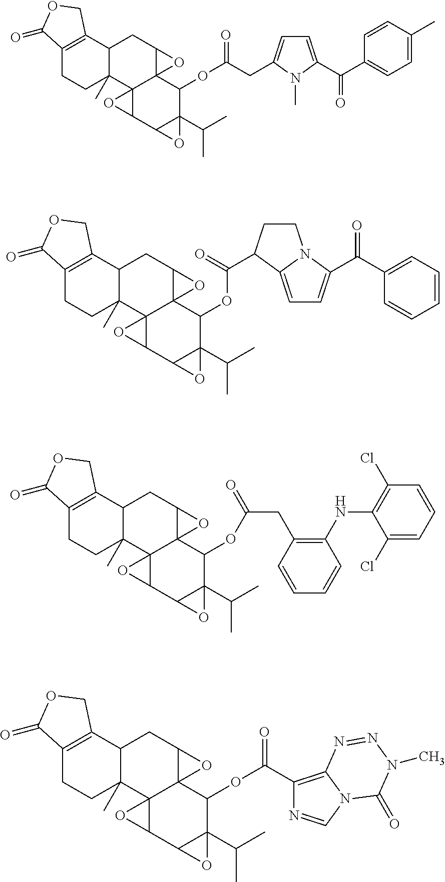 [SMILES: Cc1ccc(C(=O)c2ccc(CC(=O)OC3C4(C(C)C)OC4C4OC45C4(C)CCC6=C(COC6=O)C4CC4OC435)n2C)cc1 ; CC(C)C12OC1C1OC13C1(C)CCC4=C(COC4=O)C1CC1OC13C2OC(=O)C1CCn2c(C(=O)c3ccccc3)ccc21 ; CC(C)C12OC1C1OC13C1(C)CCC4=C(COC4=O)C1CC1OC13C2OC(=O)Cc1ccccc1Nc1c(Cl)cccc1Cl ; CC(C)C12OC1C1OC13C1(C)CCC4=C(COC4=O)C1CC1OC13C2OC(=O)c1ncn2c(=O)n(C)nnc12]

In one embodiment, an antioxidant, anti-inflammatory, or anticancer derivative of triptolide is prepared by the conjugation of a triptolide and an antioxidant, anti-inflammatory, or anticancer molecules and is represented by Formula D-II:

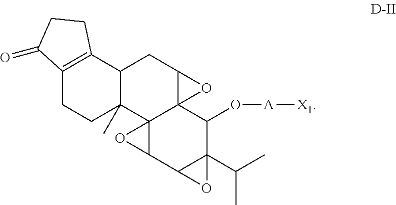

In various embodiments, A is a moiety that is formed by esterification of at least two free esterifiable hydroxyl groups on a polyol.

In various embodiments, polyols that are useful in the present invention include commercially available diols as follows:

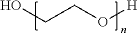

wherein n is an integer between 1 and 6;

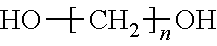

wherein n is an integer between 3 and 16;

In other embodiments, the polyols is selected from the commercial available polyols as shown below:

TABLE 1
Some exemplary polyols.

| Compound | Structure |
|---|---|
| 1 | 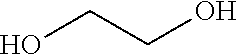 |
| 2 | 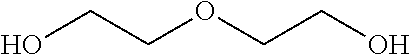 |
| 3 | 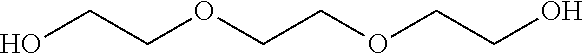 |
| 4 | 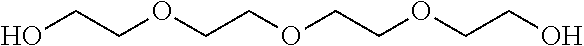 [SMILES: OCCOCCOCCOCCO] |
| 5 | 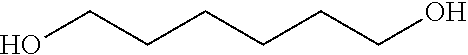 |
| 6 | 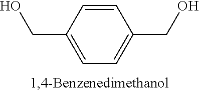 |
| 7 | 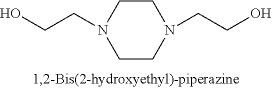 [SMILES: OCCN1CCN(CCO)CC1] |
| 8 | 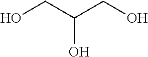 |
| 9 | 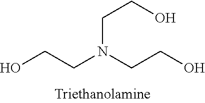 |
| 10 | 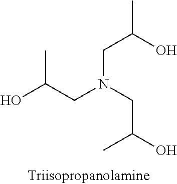 |
| 11 | 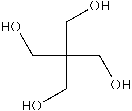 |
| 12 | 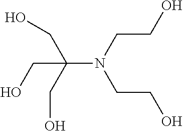 [SMILES: OCCN(CCO)C(CO)(CO)CO] |
| 13 | 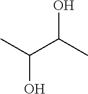 |
| 14 | 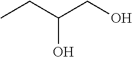 |
| 15 | 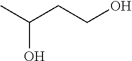 |
| 16 | 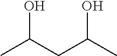 |
| 17 | 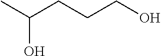 |
| 18 | 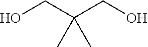 |
| 19 | 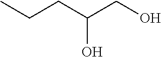 |
| 20 | 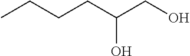 |
| 21 | 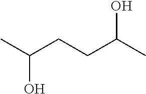 |
| 22 | 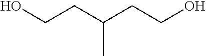 |
| 23 | 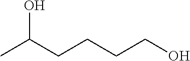 |
| 24 | 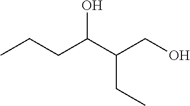 |
| 25 | 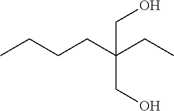 |
| 26 | 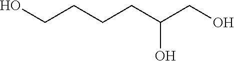 |
| 27 | 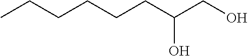 |
| 28 | 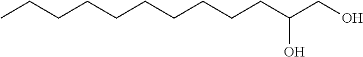 |
| 29 | 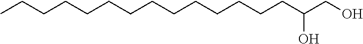 [SMILES: CCCCCCCCCCCCCCC(O)CO] |
| 30 | 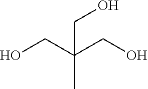 |
| 31 | 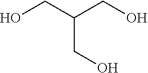 |
| 32 | 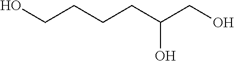 |
| 33 | 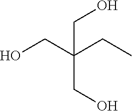 |
| 34 | 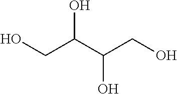 |
| 35 | 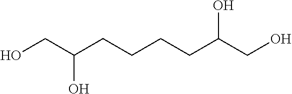 [SMILES: OCC(O)CCCCC(O)CO] |
| 36 | 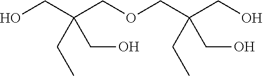 [SMILES: CCC(CO)(CO)COCC(CC)(CO)CO] |
| 37 | 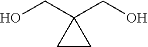 |
| 38 | 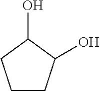 |
| 39 | 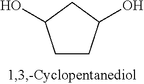 |
| 40 | 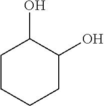 |
| 41 | 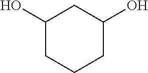 |
| 42 | 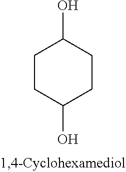 |
| 43 | 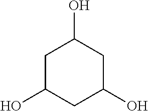 |
| 44 | 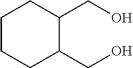 |
| 45 | 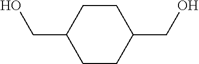 |
| 46 | 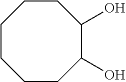 |
| 47 | 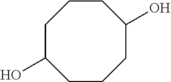 |
| 48 | 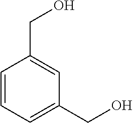 |
| 49 | 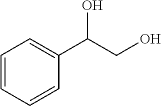 |
| 50 | 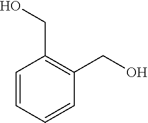 |
| 51 | 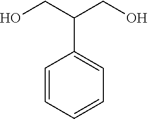 [SMILES: OCC(CO)c1ccccc1] |
| 52 | 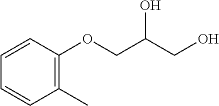 |
| 53 | 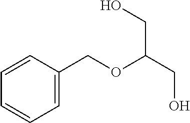 [SMILES: OCC(CO)OCc1ccccc1] |
| 54 | 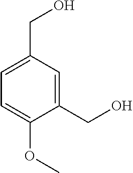 |
| 55 | 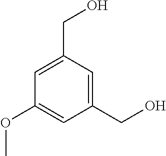 |
| 56 | 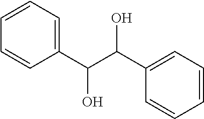 [SMILES: OC(c1ccccc1)C(O)c1ccccc1] |
| 57 | 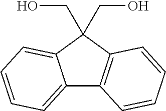 [SMILES: OCC1(CO)c2ccccc2-c2ccccc21] |
| 58 | 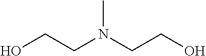 |
| 59 | 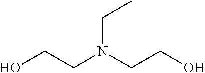 |
| 60 | 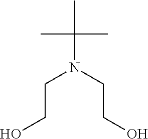 |
| 61 | 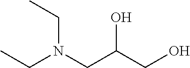 |
| 62 | 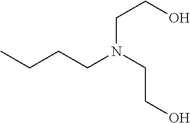 |
| 63 | 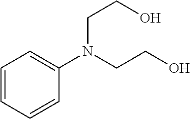 [SMILES: OCCN(CCO)c1ccccc1] |
| 64 | 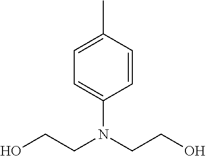 |
| 65 | 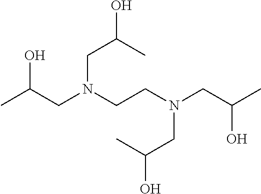 [SMILES: CC(O)CN(CCN(CC(C)O)CC(C)O)CC(C)O] |

In some embodiments, A in molecules of Formula D-I or Formula D-II can be a diamine or a moiety formed by using a diamine. In some embodiments, the diamines can NH₂—X—NH₂, wherein X can be a hydrocarbon group (for example, an alkyl, aryl, cycloaliphatic or aralkyl group), and can be saturated or unsaturated. X can also contain hetero atoms (e.g., nitrogen, oxygen, sulfur, etc.). In some other embodiments, the diamine can be NH₂(CH₂CH₂O)ₙCH₂CH₂NH₂, wherein n is an integer between 1 and 100. In still some other embodiments, diamine can be NH₂(CH₂)ₙNH₂, wherein n is an integer between 2 and 12.

In some embodiments, A in molecules of Formula D-I or Formula D-II can be an aminoalcohol or a moiety formed by using a aminoalcohol as the linker in the process of producing the NSAID derivative. Aminoalcohols that are useful in the present invention can include, but are not limited to, NH₂—Y—OH, wherein Y can be a hydrocarbon group; for example, an alkyl, aryl, cycloaliphatic or aralkyl group; and can be saturated or unsaturated. Y can also contain hetero atoms (e.g., nitrogen, oxygen, sulfur, etc.).

In some other embodiments, the aminoalcohol can be HO(CH₂CH₂O)ₙCH₂CH₂NH₂, wherein n is an integer between 1 and 100. In still some other embodiments, the aminoalcohol can be HO(CH₂)ₙNH₂, wherein n is an integer between 2 and 12.

In one embodiment, an antioxidant, anti-inflammatory derivatives of triptolide is prepared by the conjugation of a triptolide and alpha-lipoic acid and is represented by Formula D-III:

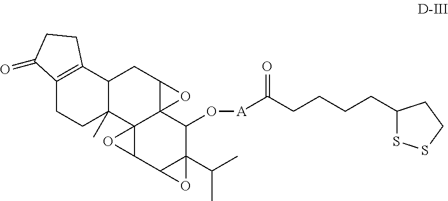

Examples of particularly useful antioxidant, anti-inflammatory derivatives of triptolide of this embodiment are represented by the following formulas:

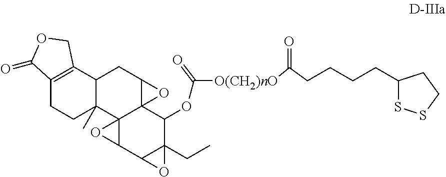

wherein n is an integer of 2-12,

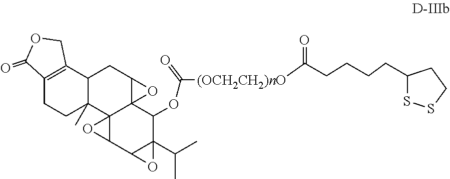

wherein n is an integer of 1-6, and

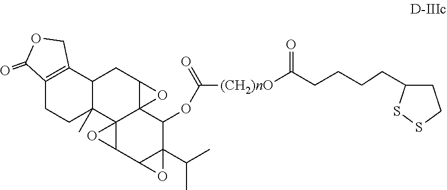

wherein n is an integer of 1-12.

In one embodiment, an anticancer derivative of triptolide is prepared by the conjugation of a triptolide and temozolomide carboxylic acid and is represented by Formula D-IV:

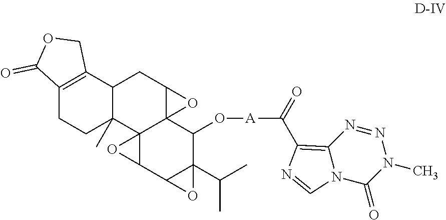

wherein the A is as described above.

Examples of particularly useful antioxidant, anti-inflammatory derivatives of triptolide of this embodiment are represented by the following formulas:

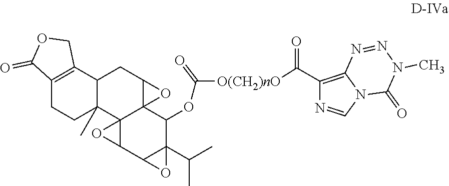

wherein n is an integer of 2-12,

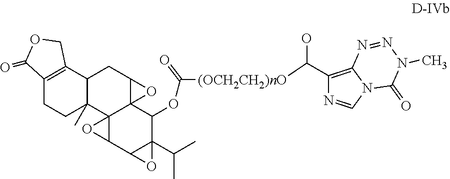

wherein n is an integer of 1-6,

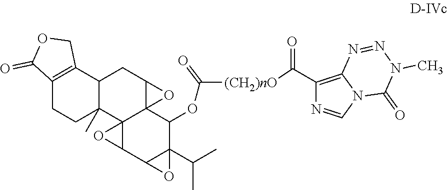

wherein n is an integer 1-12.

In one embodiment, an anticancer derivative of triptolide is prepared by the conjugation of a triptolide and a NSAID and is represented by Formula D-V:

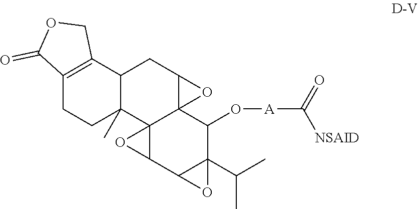

wherein the A is as described above.

In various embodiments, the NSAID is selected from the group consisting of aspirin, ibuprofen, flurbiprofen, ketoprofen, fenoprofen, fenbufen, naproxen, indomethacin, diclofenac, ketorolac, tolmetin, flufenamic acid, mefenamic acid, tolfenamic acid, meclofenamic acid, niflumic acid, sulindac, and sulindac sulfide. Examples of particularly useful antioxidant and anti-inflammatory derivatives of triptolide of this embodiment are represented by the following formulas:

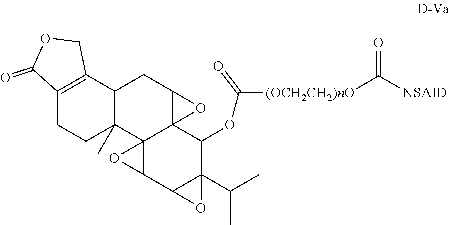

wherein n is an integer of 1-6,

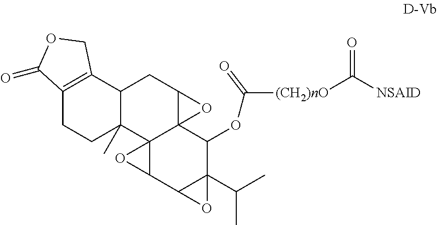

wherein n is an integer of 1-12,

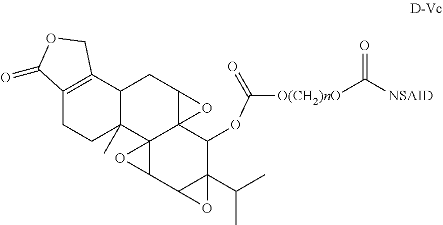

wherein n is an integer of 2-12.

In one embodiment, an anticancer derivative of triptolide is prepared by the conjugation of a triptolide and a camptothecin analog and is represented by Formula D-VI:

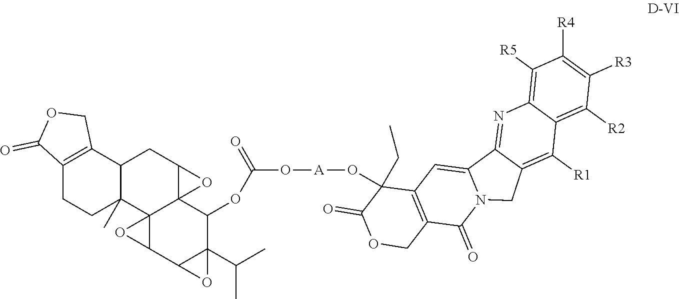

wherein the A is as described above and R1, R2, R3, R4, and R5 are independently selected from the group consisting of hydrogen, hydroxyl, alkoxy, alkyl, aryl, cycloaliphatic, acyl and aralkyl and can each optionally contain a hetero atom. Examples of particularly useful derivatives of triptolide and camptothecin analogs of this embodiment are represented by the following formulas:

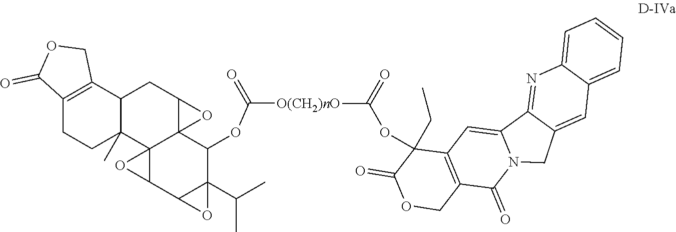

wherein n is an integer of 2-12,

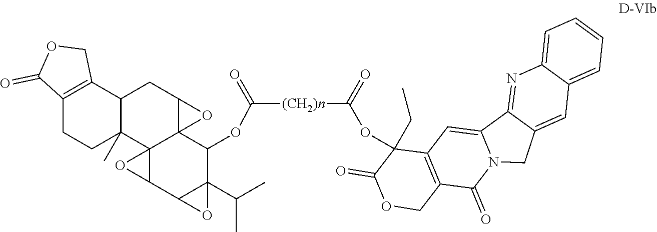

wherein n is an integer of 2-12,

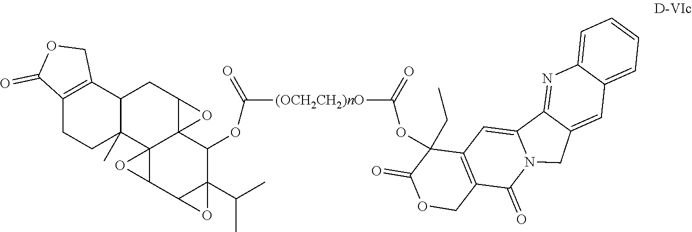

wherein n is an integer of 1-6,

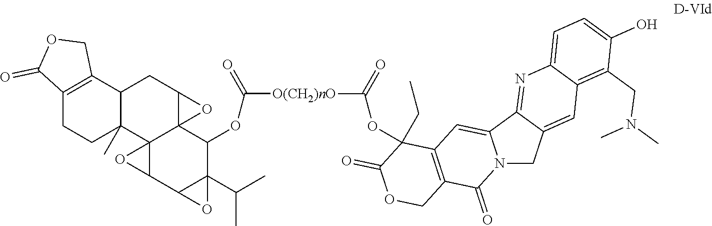

wherein n is an integer of 2-12,

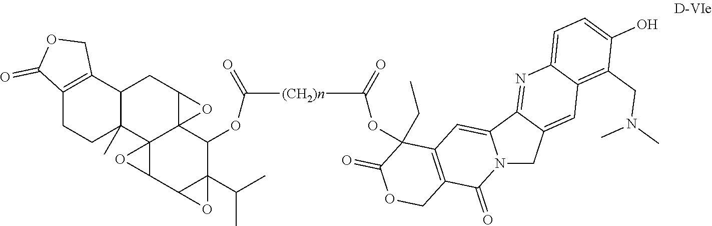

wherein n is an integer of 2-12,

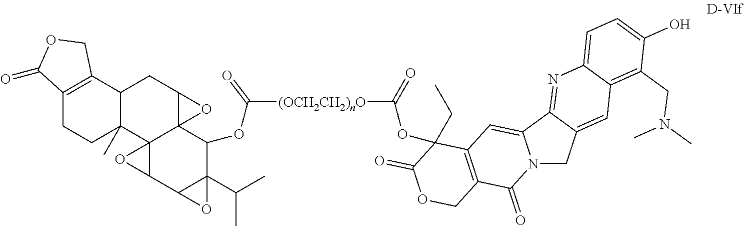

wherein n is an integer of 1-6,

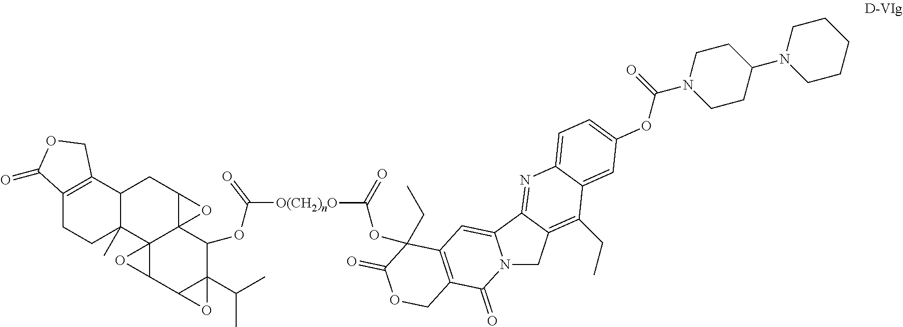

wherein n is an integer of 2-12,

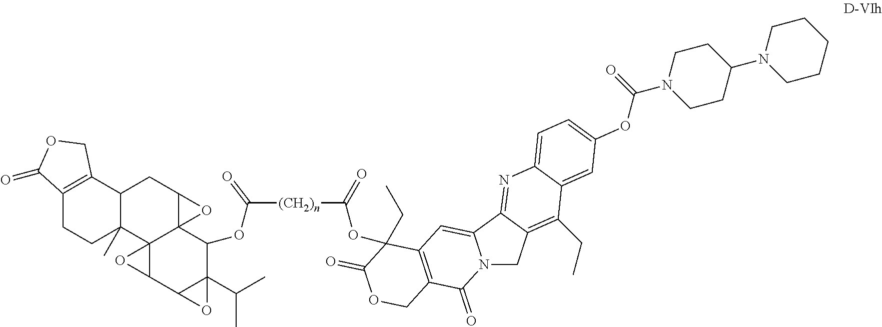

wherein n is an integer of 2-12,

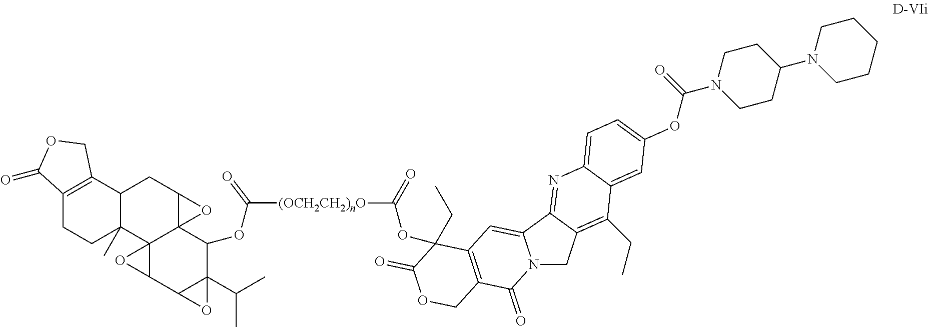

wherein n is an integer of 1-6.

In one embodiment, an anticancer derivative of triptolide is prepared by the conjugation of a triptolide and paclitaxel and is represented by Formula D-VII:

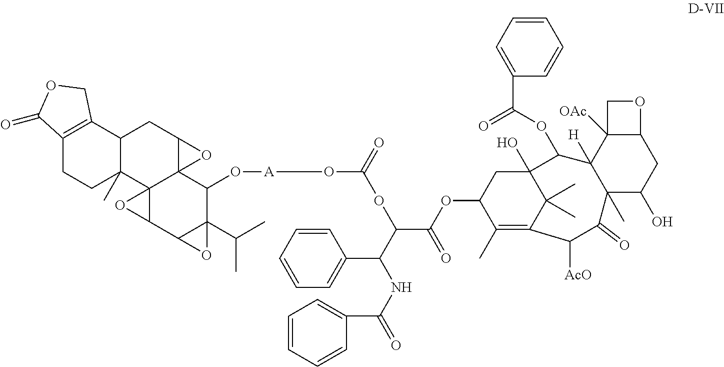

wherein A is as described above. Examples of particularly useful derivatives of triptolide and camptothecin analogs of this embodiment are represented by the following formulas:

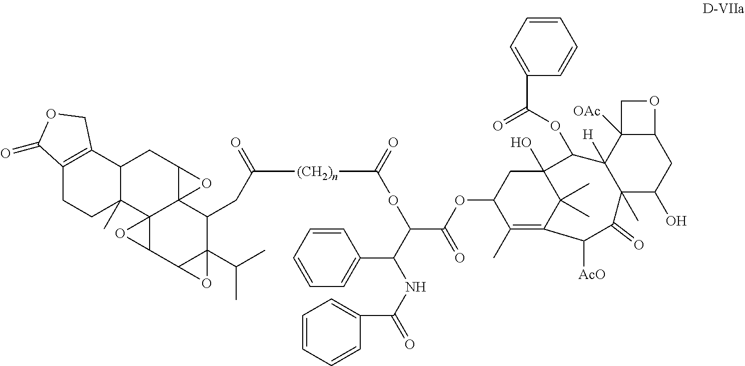

wherein n is an integer of 2-12.

In one embodiment, an anti-inflammatory derivative of triptolide is prepared by the conjugation of a triptolide and a statin lactone and is represented by Formula D-VIII:

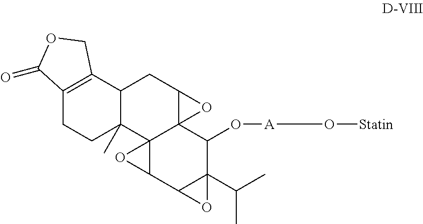

wherein A is as described above. In various embodiments, statins can be selected from the group consisting of atorvastatin, fluvastatin, rosuvastatin, lovastatin, pitavastatin, simvastatin, and mevastatin. Examples of particularly useful derivatives of triptolide and statin lactones of this embodiment are represented by the following formulas:

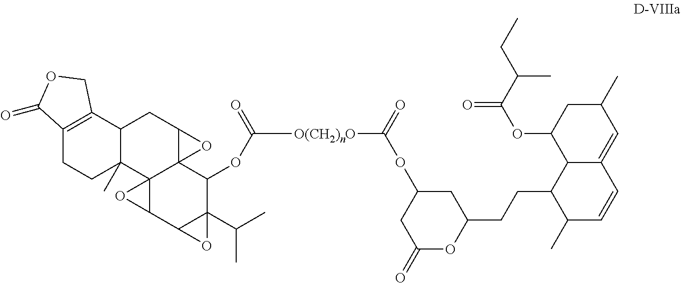

wherein n is an integer of 2-12,

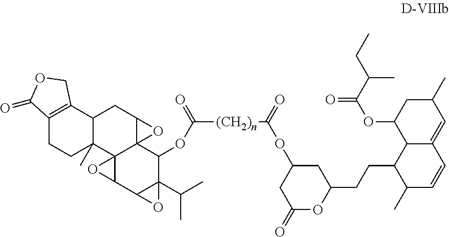

wherein n is an integer of 2-12,

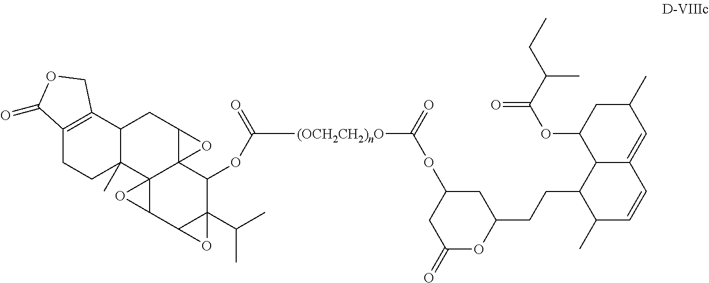

wherein n is an integer of 1-6,

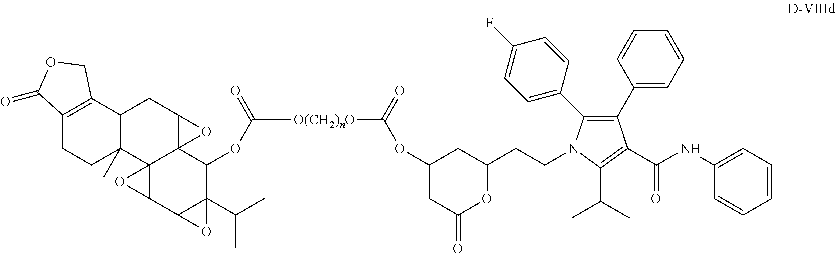

wherein n is an integer of 2-12,

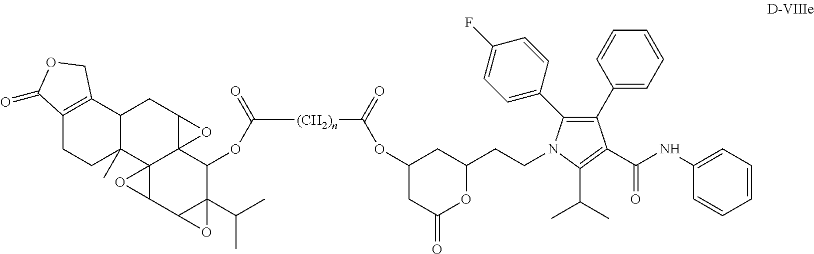

wherein n is an integer of 2-12,

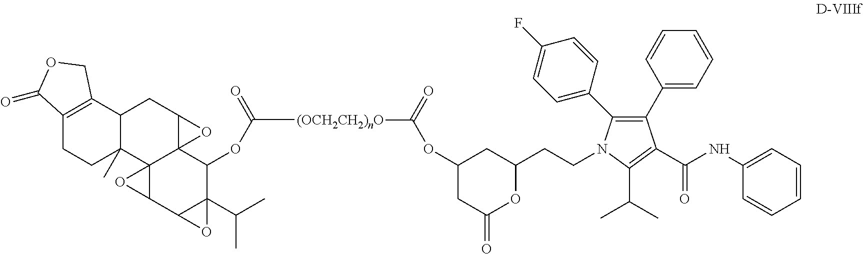

wherein n is an integer of 1-6.

Nanostructured materials have potential for precise targeting, improved tolerability, and drug efficacy (Ferrari, M. Cancer nanotechnology: opportunities and challenges. Nat. Rev. Cancer 2005, 5, 161-171.) Another advantage of nanostructured materials is that water-insoluble therapeutics can be transported more efficiently in the aqueous physiological environment when integrated into stable nanostructures (Kuo, F. et al. Nanoemulsions of an antioxidant synergy formulation containing gamma tocopherol have enhanced bioavailability and anti-inflammatory properties. Int. J. Pharm. 2008, 363, 206-213.)

Various embodiments of the present invention provide for nanospheres comprising a therapeutic agent of the invention or diagnostic agent on an amphiphilic spacer. Other embodiments of the present invention provide for nanospheres comprising a therapeutic agent or a diagnostic agent on an amphiphilic polymer.

### α-Lipoic Acid-Containing Nanospheres

In certain embodiments, the nanospheres are formed with antioxidant α-lipoic acid-containing hydrophobic compounds. Thus, in certain embodiments, the nanospheres comprise antioxidant α-lipoic acid-containing hydrophobic compounds. These compounds are disclosed in U.S. Provisional Application Ser. No. 61/018,749, filed Jan. 3, 2008, and International Application Publication No. WO 2009/086547, filed Dec. 30, 2008, which are incorporated by reference in their entirety as though fully set forth. Examples of these antioxidant α-lipoic acid-containing hydrophobic compounds include, but are not limited to the following:

Antioxidant α-lipoic acid-containing hydrophobic compounds represented by Formula A-IV

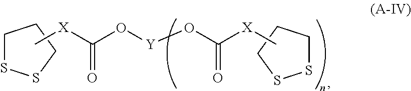

wherein X is selected from the group consisting of a substituted, unsubstituted, branched or unbranched chain of carbon atoms, and can optionally contain a heteroatom; Y is selected from the group consisting of a branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic group; and n is an integer of at least one. In particular embodiments, n is an integer from 1 to 4; and X is an unsubstituted, unbranched chain of 1 to 6 carbon atoms.

In some embodiments, the [1,2]-dithiolane moieties in molecules of formula A-IV can be independently a α-lipoic acid, and the antioxidants molecules are generally represented by the formula A-V.

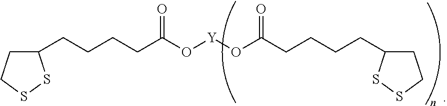

Exemplary branched or unbranched alkyl for X in molecules of formula A-IV or A-V include, but are not limited to, C1-2 alkyl, C1-3 alkyl, C1-4 alkyl, C1-6 alkyl, C1-8 alkyl, C1-10 alkyl or C1-12 alkyl. In some embodiments, A is a branched or unbranched alkyl comprising one, two, three, four, five, six, seven, eight, nine, ten, eleven, twelve or more carbons.

Exemplary branched or unbranched alkenyl for X in molecules of formula A-IV or A-V include, but are not limited to, C2-3 alkenyl, C2-4 alkenyl, C2-6 alkenyl, C2-8 alkenyl, C2-10 alkenyl or C2-12 alkenyl. In some embodiments, X is a branched or unbranched alkenyl comprising two, three, four, five, six, seven, eight, nine, ten, eleven, twelve or more carbons.

Exemplary branched or unbranched alkynyl for X in molecules of formula A-IV or A-V include, but are not limited to, C2-3 alkynyl, C2-4 alkynyl, C2-6 alkynyl, C2-8 alkynyl, C2-10 alkynyl or C2-12 alkynyl. In some embodiments, X is a branched or unbranched alkynyl comprising two, three, four, five, six, seven, eight, nine, ten, eleven, twelve or more carbons.

When X is an heteroatom-containing branched or unbranched alkyl, heteroatom-containing branched or unbranched alkenyl, or heteroatom-containing branched or unbranched alkynyl, each heteroatom can be selected independently from N, O, and S. Accordingly, in some embodiments, the heteroatom is N. In some embodiments, heteroatom is O. In some embodiments, the heteroatom is S.

Exemplary branched or unbranched alkyl for Y in molecules of formula A-IV or A-V include, but are not limited to, C1-2 alkyl, C1-3 alkyl, C1-4 alkyl, C1-6 alkyl, C1-8 alkyl, C1-10 alkyl or C1-12 alkyl. In some embodiments, Y is a branched or unbranched alkyl comprising one, two, three, four, five, six, seven, eight, nine, ten, eleven, twelve or more carbons.

Exemplary branched or unbranched alkenyl for Y in molecules of formula A-IV or A-V include, but are not limited to, C2-3 alkenyl, C2-4 alkenyl, C2-6 alkenyl, C2-8 alkenyl, C2-10 alkenyl or C2-12 alkenyl. In some embodiments, Y is a branched or unbranched alkenyl comprising two, three, four, five, six, seven, eight, nine, ten, eleven, twelve or more carbons.

Exemplary branched or unbranched alkynyl for Y in molecules of formula A-IV or A-V include, but are not limited to, C2-3 alkynyl, C2-4 alkynyl, C2-6 alkynyl, C2-8 alkynyl, C2-10 alkynyl or C2-12 alkynyl. In some embodiments, A is a branched or unbranched alkynyl comprising two, three, four, five, six, seven, eight, nine, ten, eleven, twelve or more carbons.

When the Y is an heteroatom-containing branched or unbranched alkyl, heteroatom-containing branched or unbranched alkenyl, or heteroatom-containing branched or unbranched alkynyl, each heteroatom can be selected independently from N, O, and S. Accordingly, in some embodiments, the heteroatom is N. In some embodiments, heteroatom is O. In some embodiments, the heteroatom is S.

Exemplary cyclic aliphatic for Y in molecules of formula A-IV or A-V include, but are not limited to C3-12 cyclic aliphatic. For example, the cyclic aliphatic can be a C3, C4, C5, C6, C7, or C8 cyclic aliphatic. In some embodiments, the cyclic aliphatic is C8-12 cyclic aliphatic.

Exemplary cyclic aromatics for Y in molecules of formula A-IV or A-V include, but are not limited to C4-12 cyclic aromatics. For example, the cyclic aromatic can be a C4, C5, C6, C7, or C8 cyclic aromatic. In some embodiments, the cyclic aromatic is C8-12 cyclic aromatic.

Exemplary heterocyclic for Y in molecules of formula A-IV or A-V include, but are not limited to C4-12 heterocyclic. For example, the cyclic aromatic can be a C4, C5, C6, C7, or C8 heterocyclic. In some embodiments, the heterocyclic is C8-12 cyclic heterocyclic.

In various embodiments, Y in molecules of formula A-IV or A-V can be a polyol or a moiety that is formed by esterification of at least two free esterifiable hydroxyl groups on a polyol. In some embodiments, the polyol can be HO(CH₂CH₂O)ₙH, wherein n on the polyol can be an integer between 1 and 6. In some embodiments, the polyol can be HO(CH₂)ₙH, wherein n on the polyol can be an integer between 3 and 16.

In other embodiments, Y in molecules of formula A-IV or A-V can be or formed from esterification of a polyol selected from group consisting of an ethylene glycol, diethylene glycol, triethylene glycol, tetraethylene glycol, pentaethylene glycol, hexaethylene glycol, 1,3-propanediol, and 1,4-butanediol. In some embodiments, Y in molecules of formula A-IV or A-V can be a polyol selected from those listed in Table 1.

In some embodiments, Y in molecules of formula A-IV or A-V can be a diamine or a moiety formed by using a diamine. In some embodiments, the diamines can NH₂—X—NH₂, wherein X can be a hydrocarbon group (for example, an alkyl, aryl, cycloaliphatic or aralkyl group), and can be saturated or unsaturated. Y can also contain hetero atoms (e.g., nitrogen, oxygen, sulfur, etc.). In some other embodiments, the diamine can be NH₂(CH₂CH₂O)ₙCH₂CH₂NH₂, wherein n is an integer between 1 and 100. In still some other embodiments, diamine can be NH₂(CH₂)ₙNH₂, wherein n is an integer between 2 and 12.

In some embodiments, Y in molecules of formula A-IV or A-V can be an aminoalcohol or a moiety formed by using a aminoalcohol as the linker in the process of producing the compound of Formula A-IV or A-V. Aminoalcohols that are useful in the present invention can include, but are not limited to, NH₂—Z—OH, wherein Z can be a hydrocarbon group; for example, an alkyl, aryl, cycloaliphatic or aralkyl group; and can be saturated or unsaturated. Y can also contain hetero atoms (e.g., nitrogen, oxygen, sulfur, etc.). In some other embodiments, the aminoalcohol can be HO(CH₂CH₂O)ₙCH₂CH₂NH₂, wherein n is an integer between 1 and 100. In still some other embodiments, the aminoalcohol can be HO(CH₂)ₙNH₂, wherein n is an integer between 2 and 12.

In various embodiments, Y can be a moiety formed by esterification of the hydroxyl groups of a polyol. In various embodiments, the polyol is selected from the group consisting of

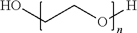

wherein n is an integer between 1 and 4 and

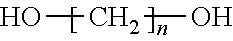

wherein n is an integer between 3 and 16.

One example of a particularly useful multiple α-lipoic acid-containing hydrophobic compound is represented as follows:

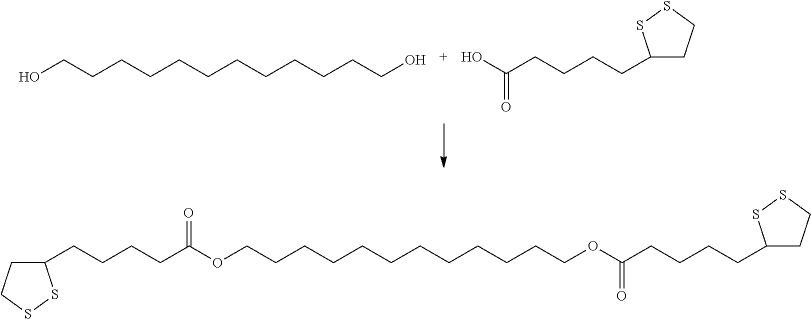 [SMILES: OCCCCCCCCCCCCO ; O=C(O)CCCCC1CCSS1 ; O=C(CCCCC1CCSS1)OCCCCCCCCCCCCOC(=O)CCCCC1CCSS1]

Various embodiments of the present invention provide for methods of using the nanospheres described herein. Various embodiments of the present invention provide for methods of using the nanospheres comprising a therapeutic agent or diagnostic agent on an amphiphilic spacer describe herein. Various embodiments of the present invention provide for methods of using nanospheres comprising a therapeutic agent or a diagnostic agent on an amphiphilic polymer as described herein. Methods of using these nanospheres include administering a nanosphere of the present invention to a subject in need of treatment for cancer.

### TPL-Derivative (TPLD) Nanospheres

In certain embodiments, the nanospheres are formed with hydrophobic TPL derivatives. Thus, in certain embodiments, the nanospheres comprise hydrophobic TPL derivatives. In certain embodiments, the nanospheres are formed with hydrophobic antioxidant, anti-inflammatory and anticancer derivatives of TPL. Thus, in certain embodiments, the nanospheres comprise hydrophobic antioxidant, anti-inflammatory, and anticancer derivatives of TPL. In various embodiments, the nanospheres are antioxidant nanospheres.

In certain embodiments, the nanospheres are formed with tocopherol. Thus, in certain embodiments, the TPLD nanospheres comprise tocopherol.

In one embodiment, an antioxidant derivative of TLP and/or an antioxidant derivative of a TLP analog can be represented by Formula D-IX:

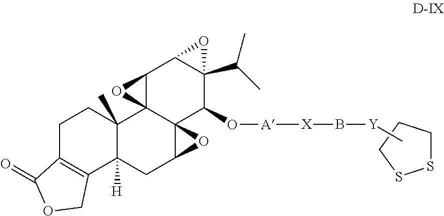

wherein A′ and B are independently selected from the group consisting of —OC(O)—, —OC(O)O—, and —OC(O)N(R)—, wherein R is a hydrogen atom, or a substituted, unsubstituted, branched or unbranched chain of carbon atoms and can contain heteroatoms (e.g., nitrogen, oxygen, sulfur, etc.); wherein X and Y can be each be a linker that can be a substituted, unsubstituted, branched or unbranched chain of carbon atoms and can contain heteroatoms (e.g., nitrogen, oxygen, sulfur, etc.).

In one embodiment, an antioxidant derivative of TLP and/or antioxidant derivative of a TLP analog is prepared by the conjugation of a TLP or a TLP analog and an α-lipoic acid and is represented by Formula D-IXa:

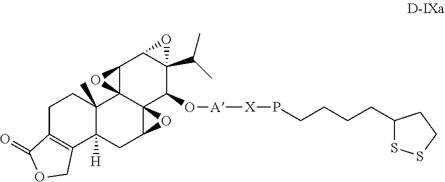

wherein A′ is selected from the group consisting of —OC(O)—, —OC(O)O—, and —OC(O)N(R)—, wherein R is a hydrogen atom, or a substituted, unsubstituted, branched or unbranched chain of carbon atoms and can contain heteroatoms (e.g., nitrogen, oxygen, sulfur, etc.); wherein P is selected from the group consisting of —OC(O)—, and —N(R)C(O)—, wherein R is a hydrogen atom, or a substituted, unsubstituted, branched or unbranched chain of carbon atoms and can contain heteroatoms (e.g., nitrogen, oxygen, sulfur, etc.); wherein X can be a linker that can be a substituted, unsubstituted, branched or unbranched chain of carbon atoms and can contain heteroatoms (e.g., nitrogen, oxygen, sulfur, etc.).

In another embodiment, an antioxidant derivative of TLP and/or antioxidant derivative of a TLP analog is prepared by the conjugation of TLP or a TLP analog and α-lipoic acid via a diol and is represented Formula D-IXb:

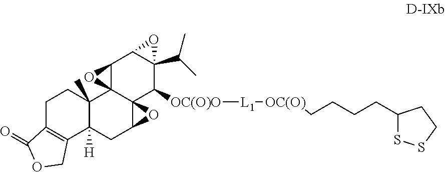

wherein L₁ can be a moiety formed by esterification of two free esterifiable hydroxyl groups on a diol.

In various embodiments, diols that are useful in the present invention can be represented by the following formula:

HO-W-OH

wherein W can be a hydrocarbon group; for example, an alkyl, aryl, cycloaliphatic or aralkyl group; and can be saturated or unsaturated. W can also contain hetero atoms (e.g., nitrogen, oxygen, sulfur, etc.).

Additional examples of diols that are useful in the present invention include, but are not limited to commercially available one as follows:

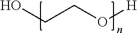

wherein n is an integer between 1 and 100.

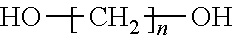

wherein n is an integer between 2 and 12.

### 1,4-Bis(2-hydroxyethyl)-piperazine

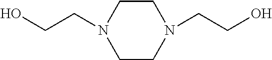 [SMILES: OCCN1CCN(CCO)CC1]

### 1,3-Cyclopentanediol

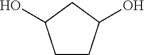

### 1,4-cyclohexanediol

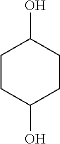

Examples of particularly useful antioxidant derivatives of TLP and/or antioxidant derivatives of TLP analogs of this embodiment are represented by the following formulas:

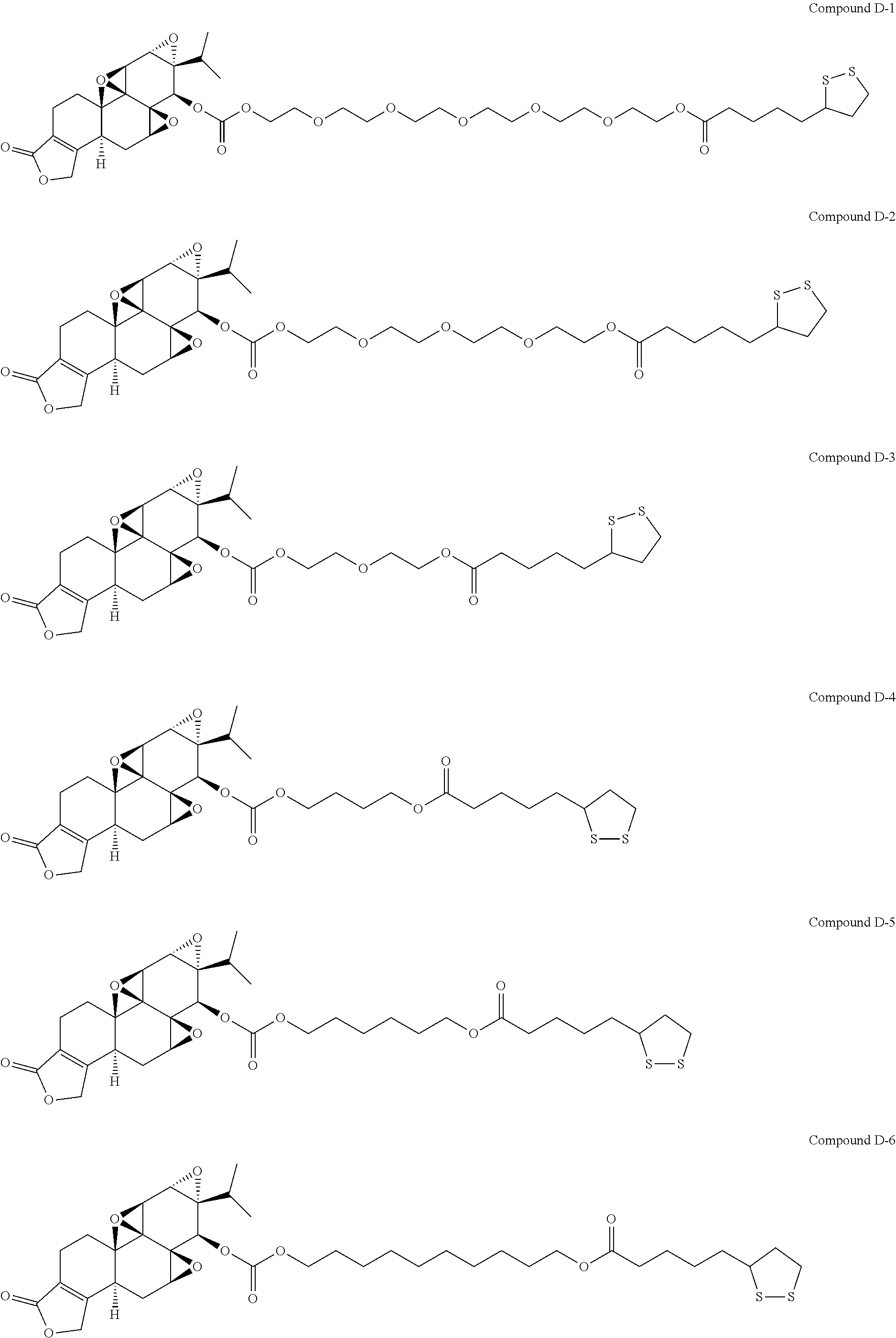

One exemplary compound and its synthesis are shown below.

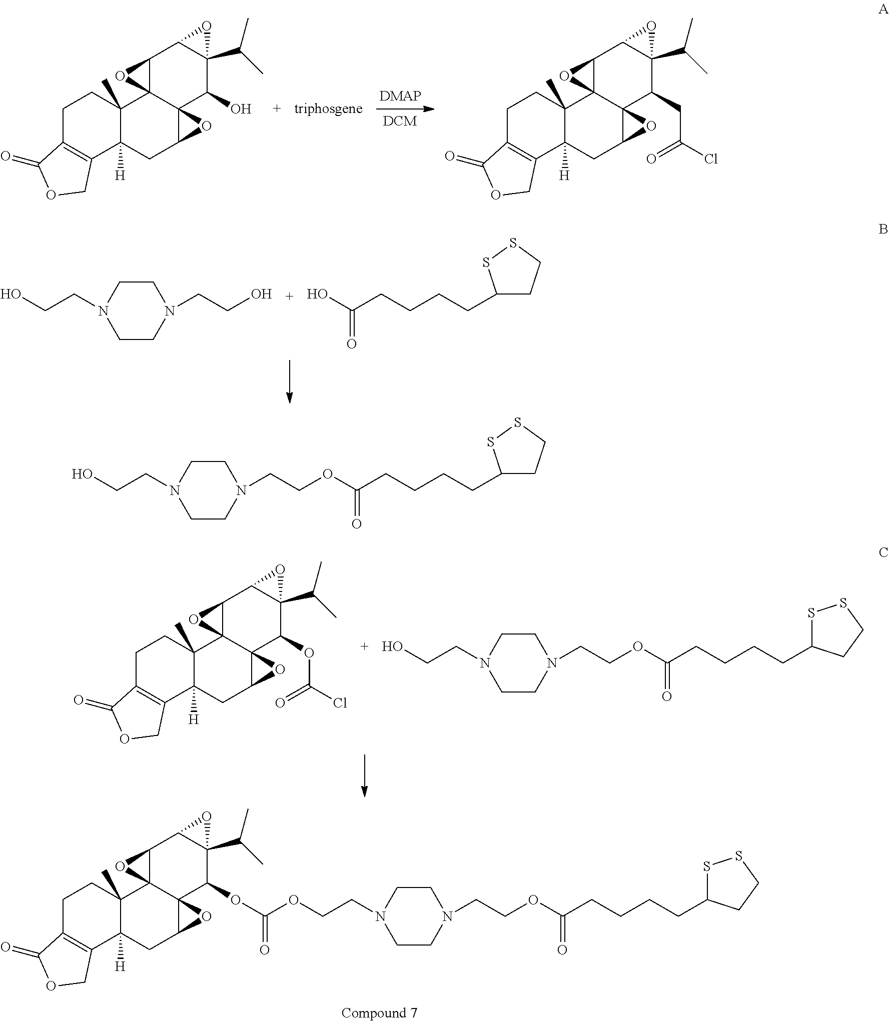

In another embodiment, an antioxidant derivative of a TLP and/or antioxidant derivative of a TLP analog is prepared by the conjugation of TLP or a TLP analog and an α-lipoic acid via a diamine and is represented by Formula D-IXc:

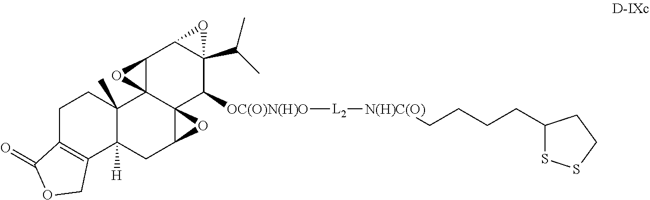

wherein L₂ can be a moiety formed by using a diamine as the linker in the process of producing the antioxidant TLP derivative or the antioxidant TLP analog derivative.

In one embodiment, diamines that are useful in the present invention can be represented by the following formula:

N₂N—X—NH₂

wherein X can be a hydrocarbon group; for example, an alkyl, aryl, cycloaliphatic or aralkyl group; and can be saturated or unsaturated. X can also contain hetero atoms (e.g., nitrogen, oxygen, sulfur, etc.).

In other embodiments, diamines that are useful in the present inventive compounds include, but are not limited to commercially available ones as follows:

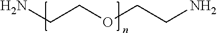

wherein n is an integer between 1 and 100.

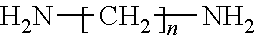

wherein n is an integer between 2 and 12.

Examples of particularly useful antioxidant derivatives of TLP and/or antioxidant derivatives of TLP analogs of this embodiment are represented by the following formulas:

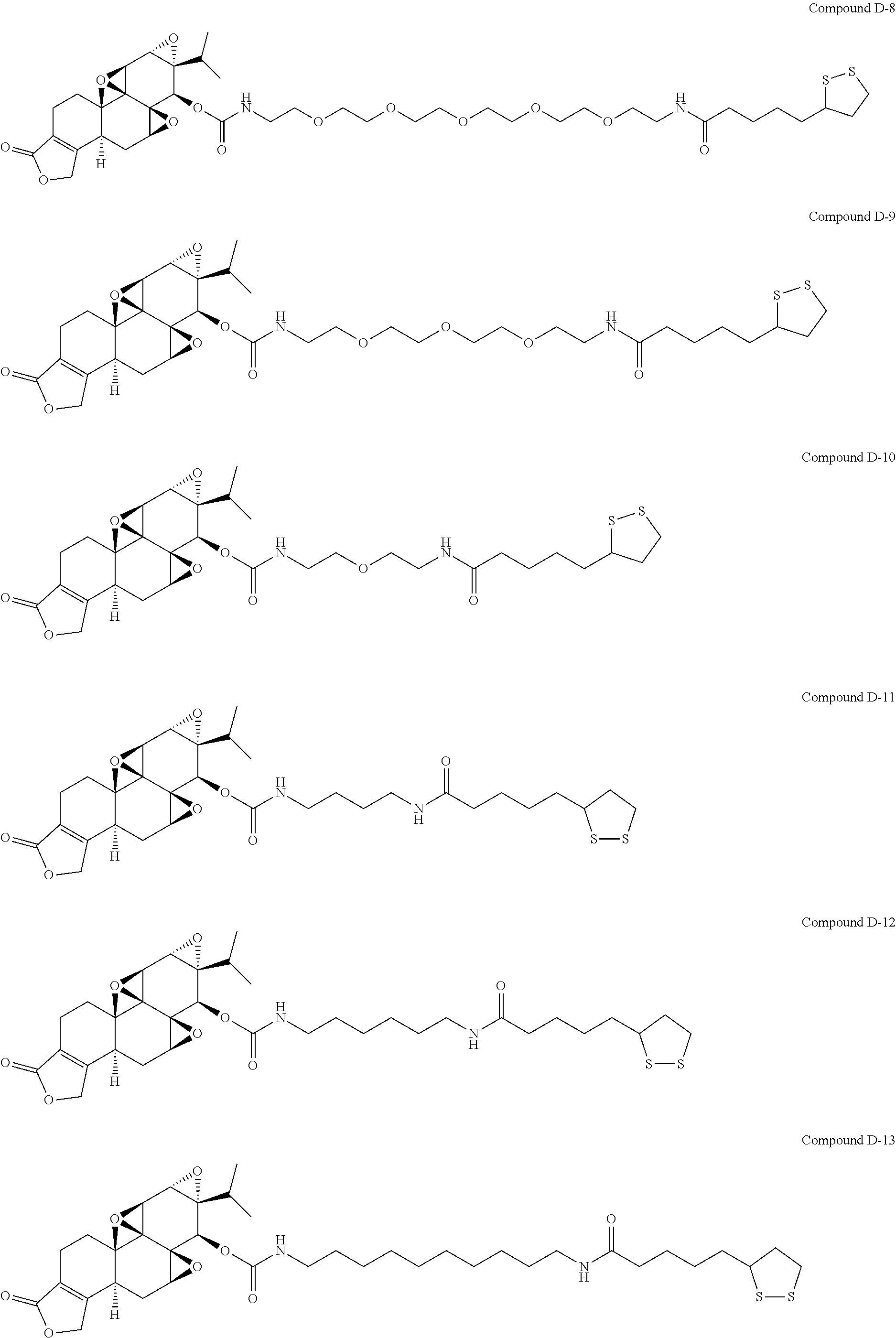

One exemplary compound and its synthesis are shown below.

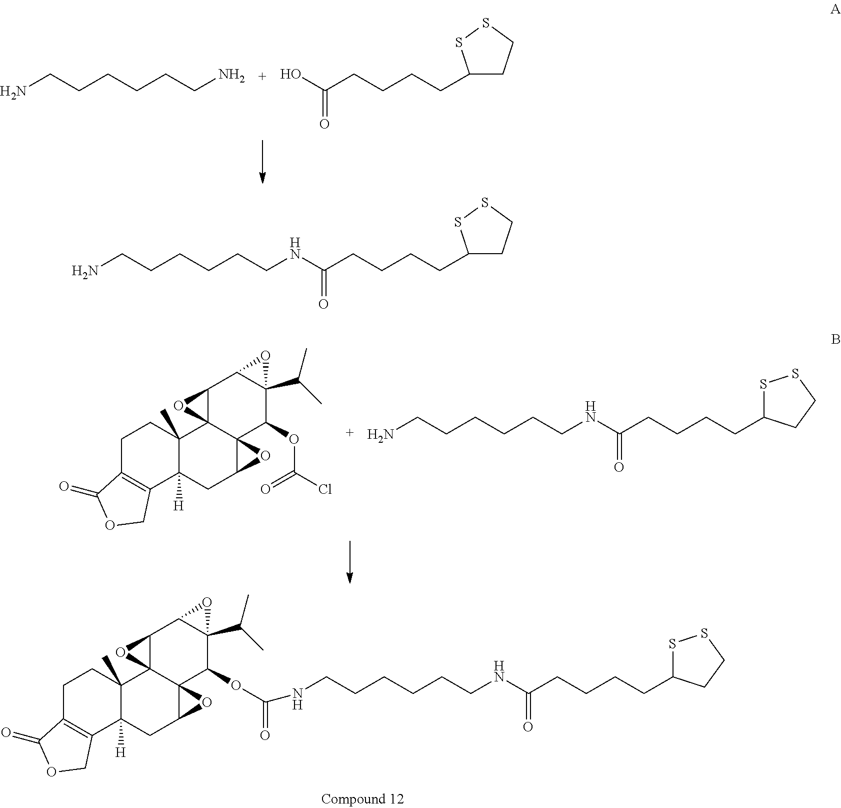 [SMILES: NCCCCCCN ; O=C(O)CCCCC1CCSS1 ; NCCCCCCNC(=O)CCCCC1CCSS1 ; CC(C)[C@]12O[C@H]1[C@@H]1O[C@]13[C@]1(O[C@H]1C[C@H]1C4=C(CC[C@@]13C)C(=O)OC4)[C@@H]2OC(=O)Cl ; NCCCCCCNC(=O)CCCCC1CCSS1 ; CC(C)[C@]12O[C@H]1[C@@H]1O[C@]13[C@]1(O[C@H]1C[C@H]1C4=C(CC[C@@]13C)C(=O)OC4)[C@@H]2OC(=O)NCCCCCCNC(=O)CCCCC1CCSS1]

In another embodiment, an antioxidant derivative of TLP and/or antioxidant derivative of a TLP analog is prepared by the conjugation of TLP or a TLP analog and an α-lipoic acid via an aminoalcohol and is represented by Formula D-IXd:

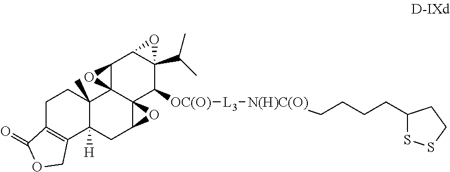

wherein L₃ can be a moiety formed by using an aminoalcohol as the linker in the process of producing the antioxidant TLP derivative or the antioxidant TLP analog derivative.

Aminoalcohols that are useful in the present invention can be represented by the following formula:

H₂N—Y—OH

wherein Y can be a hydrocarbon group; for example, an alkyl, aryl, cycloaliphatic or aralkyl group; and can be saturated or unsaturated. Y can also contain hetero atoms (e.g., nitrogen, oxygen, sulfur, etc.).

Examples of aminoalcohols that are useful in the present inventive compounds include, but are not limited to commercially available one as follows:

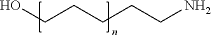

wherein n is an integer between 1 and 100.

HOCH₂ₙNH₂

wherein n is an integer between 2 and 12.

Examples of particularly useful antioxidant derivatives of TLP and/or antioxidant derivatives of TLP analogs of this embodiment are represented by the following formulas:

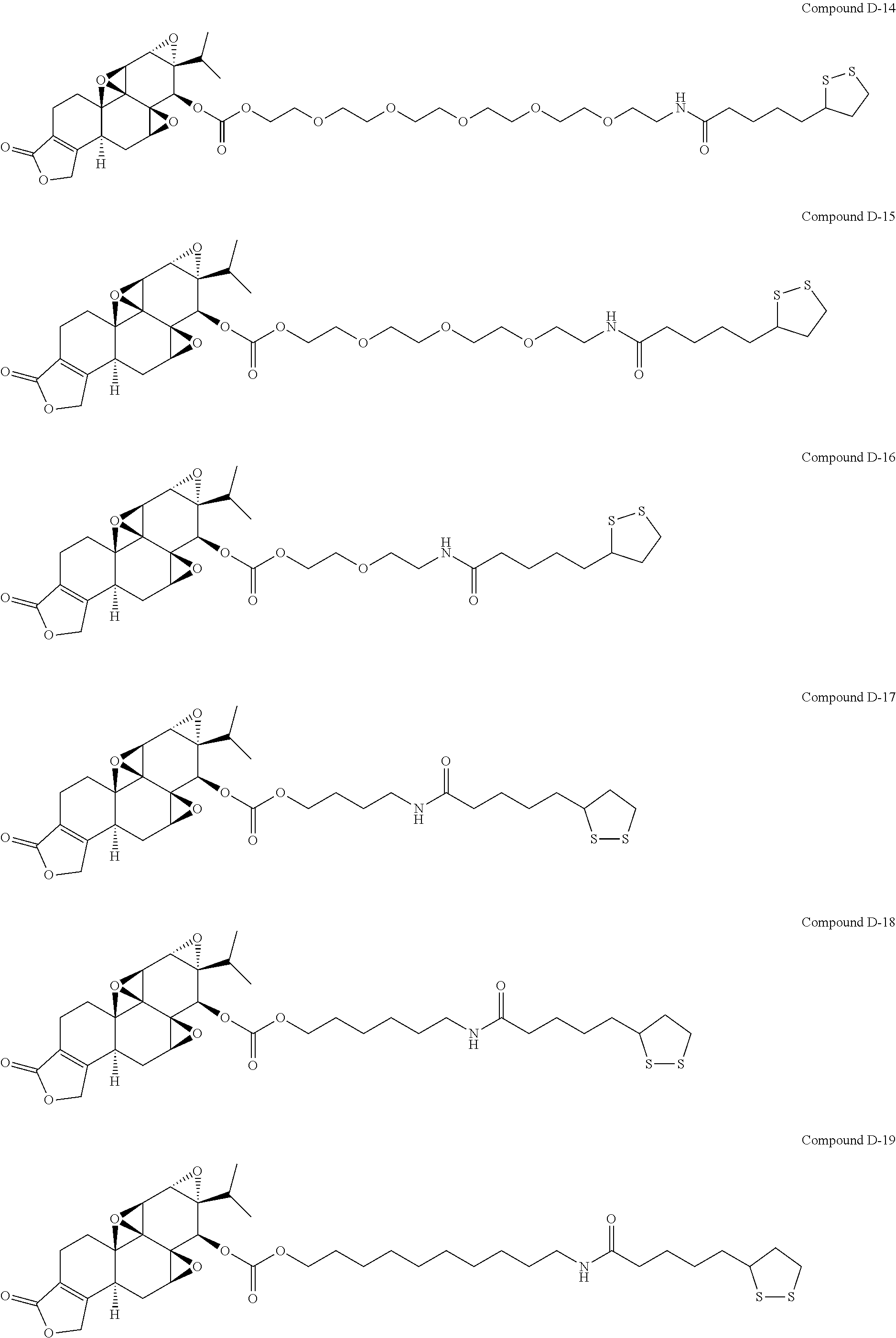

One exemplary compound and its synthesis are shown below.

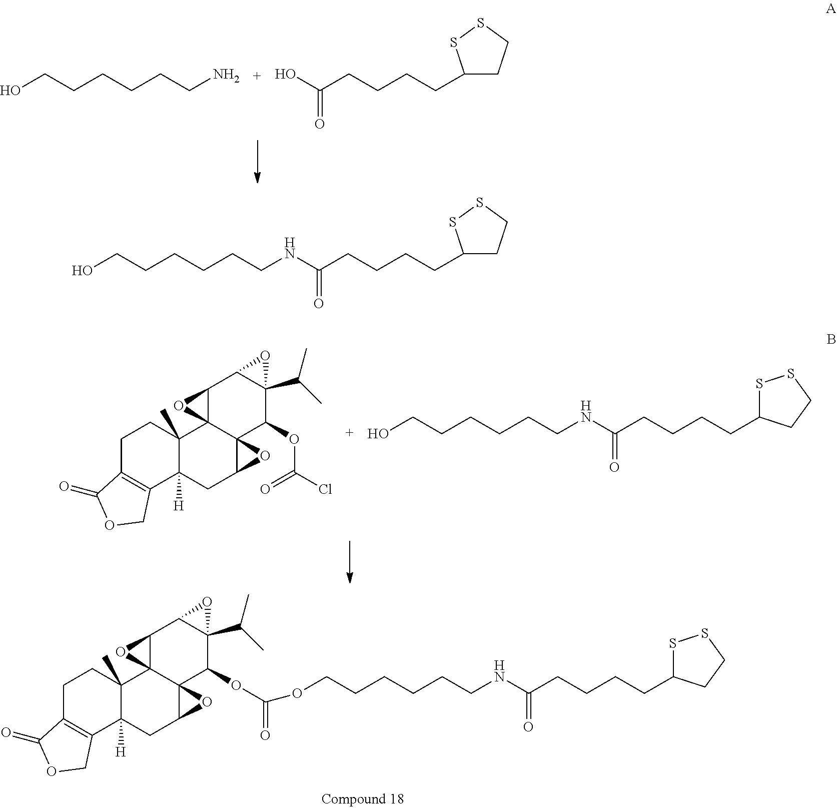

Additional embodiments of the present invention provide for the following compounds:

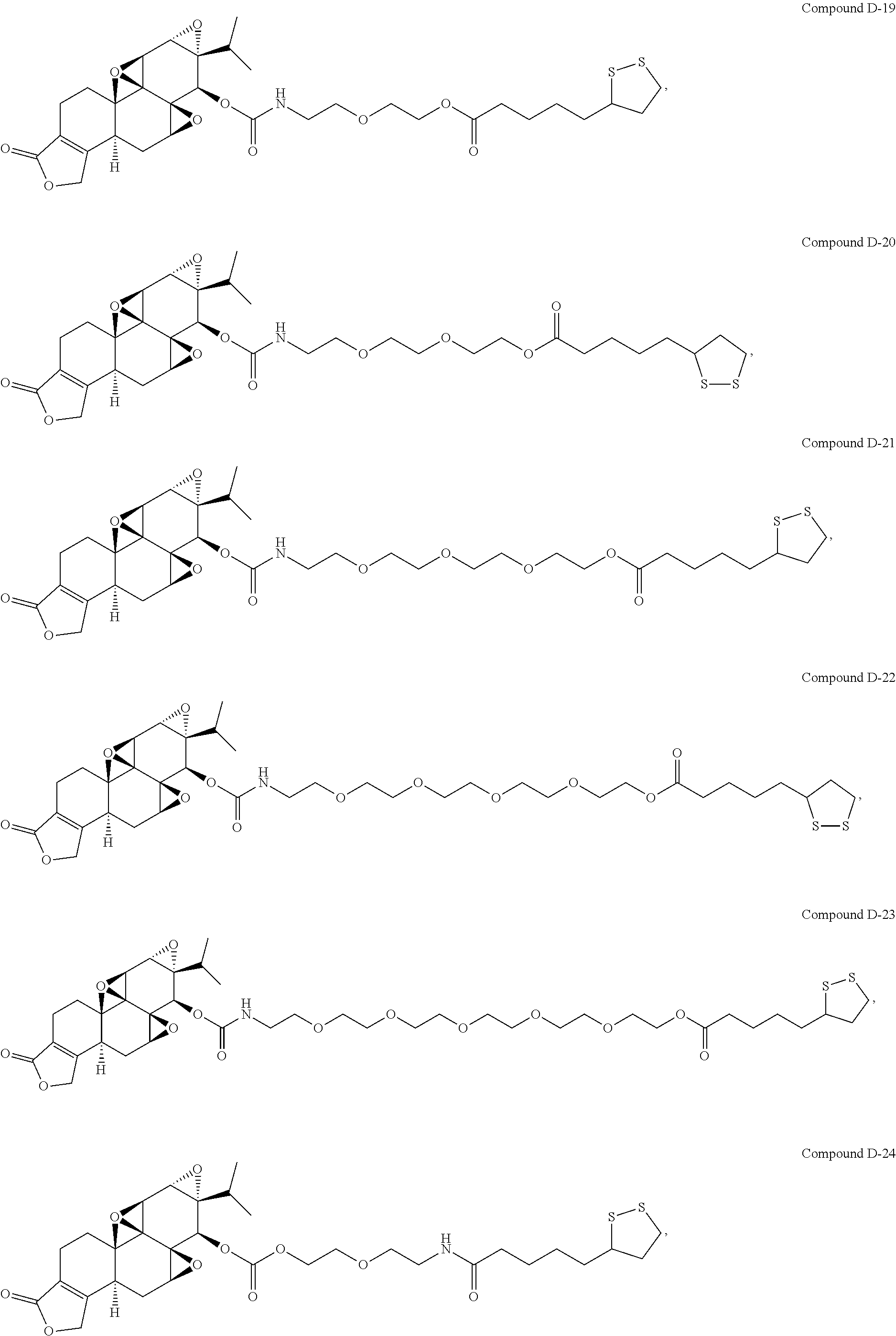

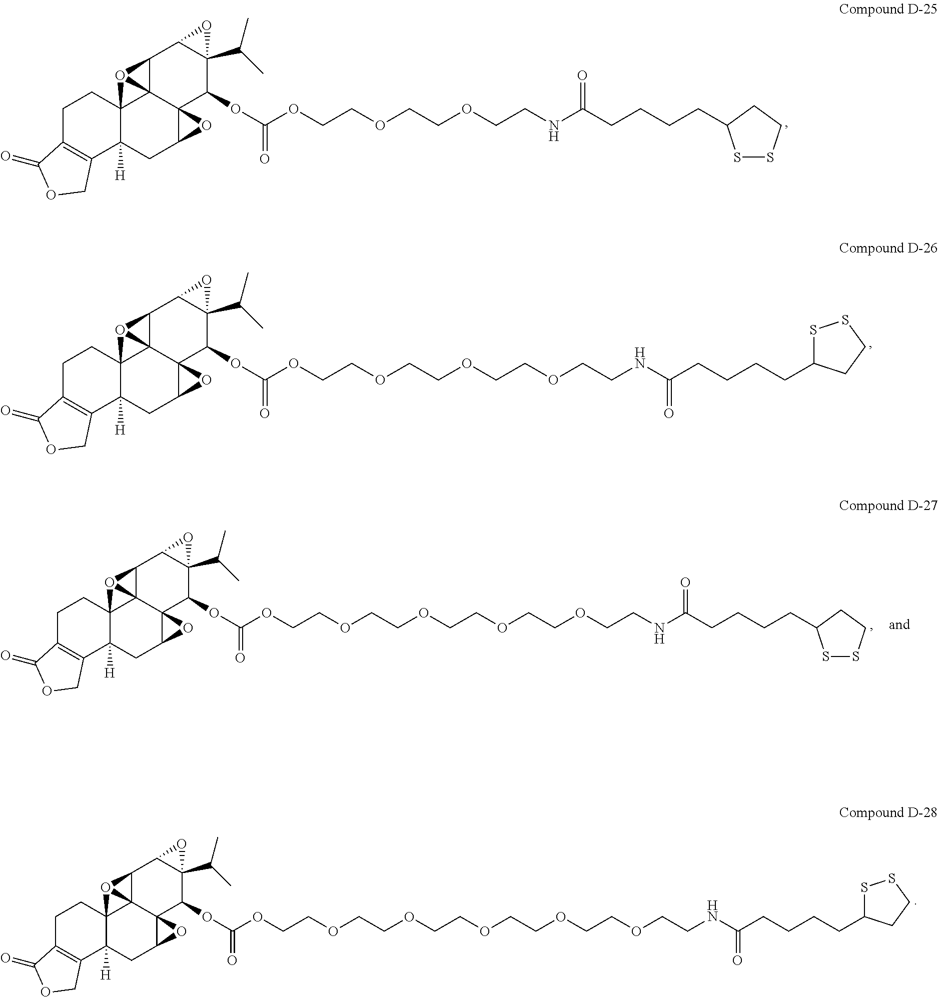

In another embodiment, the TLP analogs are modified by reaction with succinic anhydride or glutaric anhydride and an antioxidant derivative of TLP and/or antioxidant derivative of a TLP analog is prepared by the conjugation of an α-lipoic acid and the modified TLP or TLP analog. One exemplary compound and its synthesis are shown below.

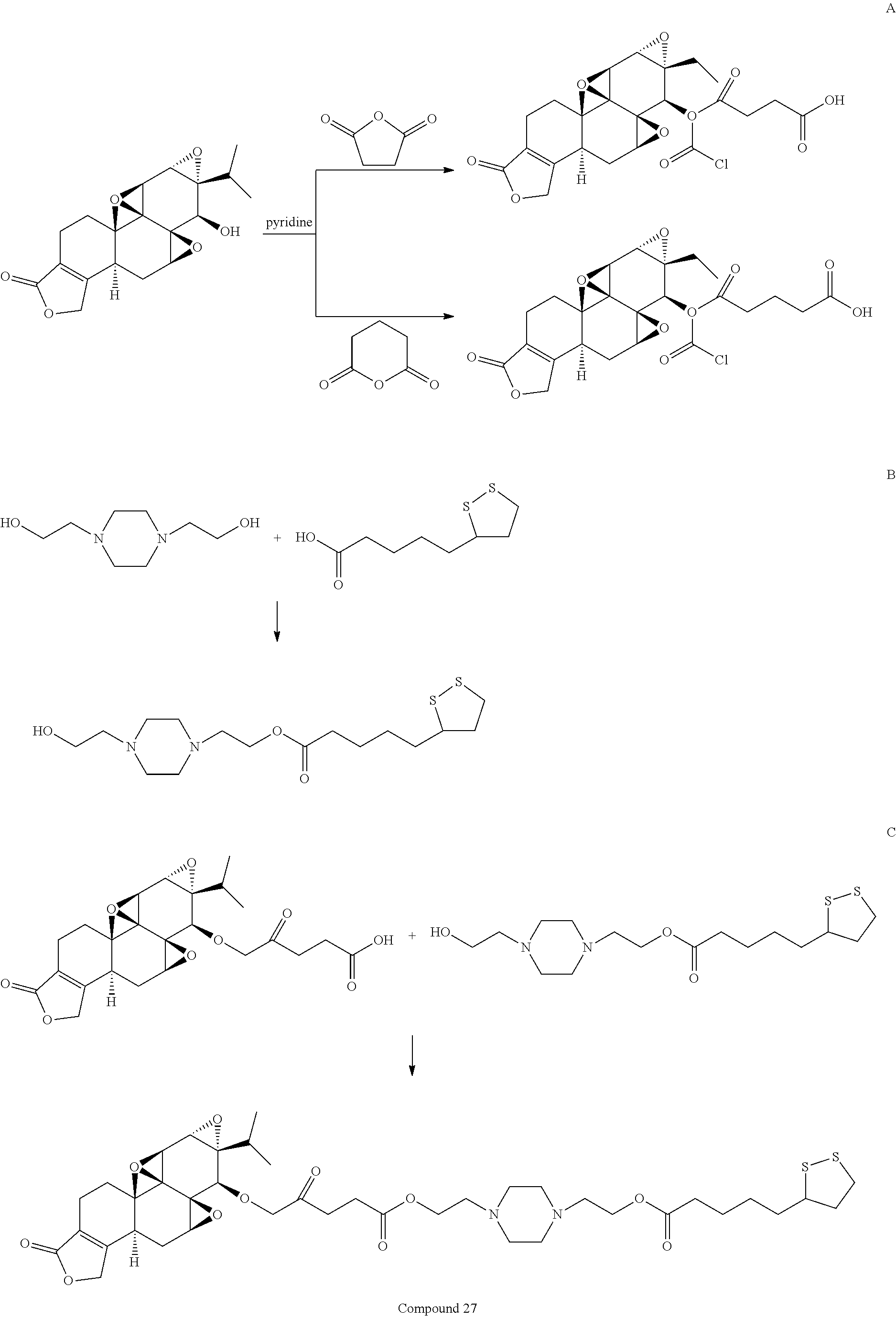

Additional examples of particularly useful antioxidant derivatives of TLP and/or antioxidant derivatives of TLP analogs are represented by formulas as follows:

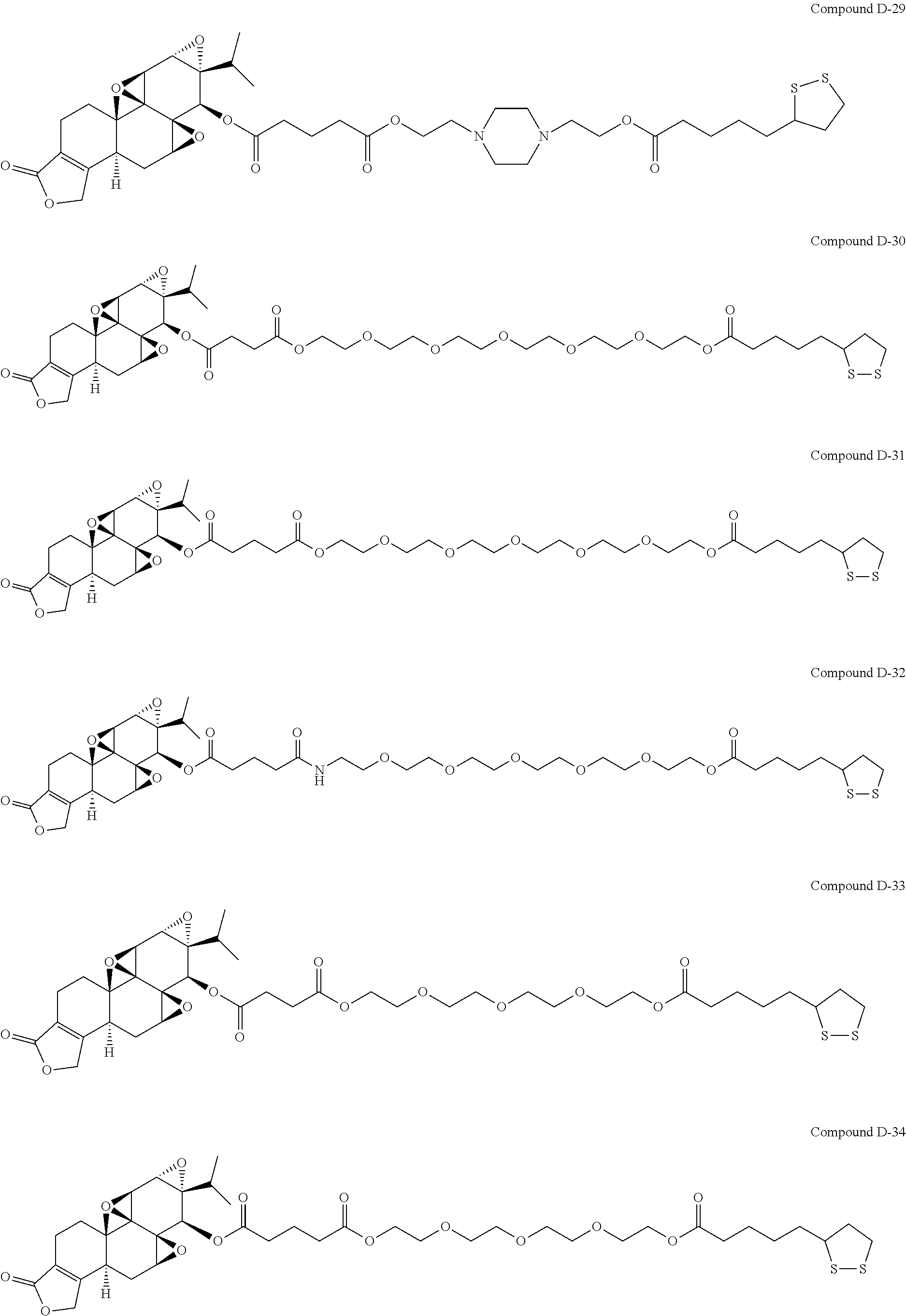

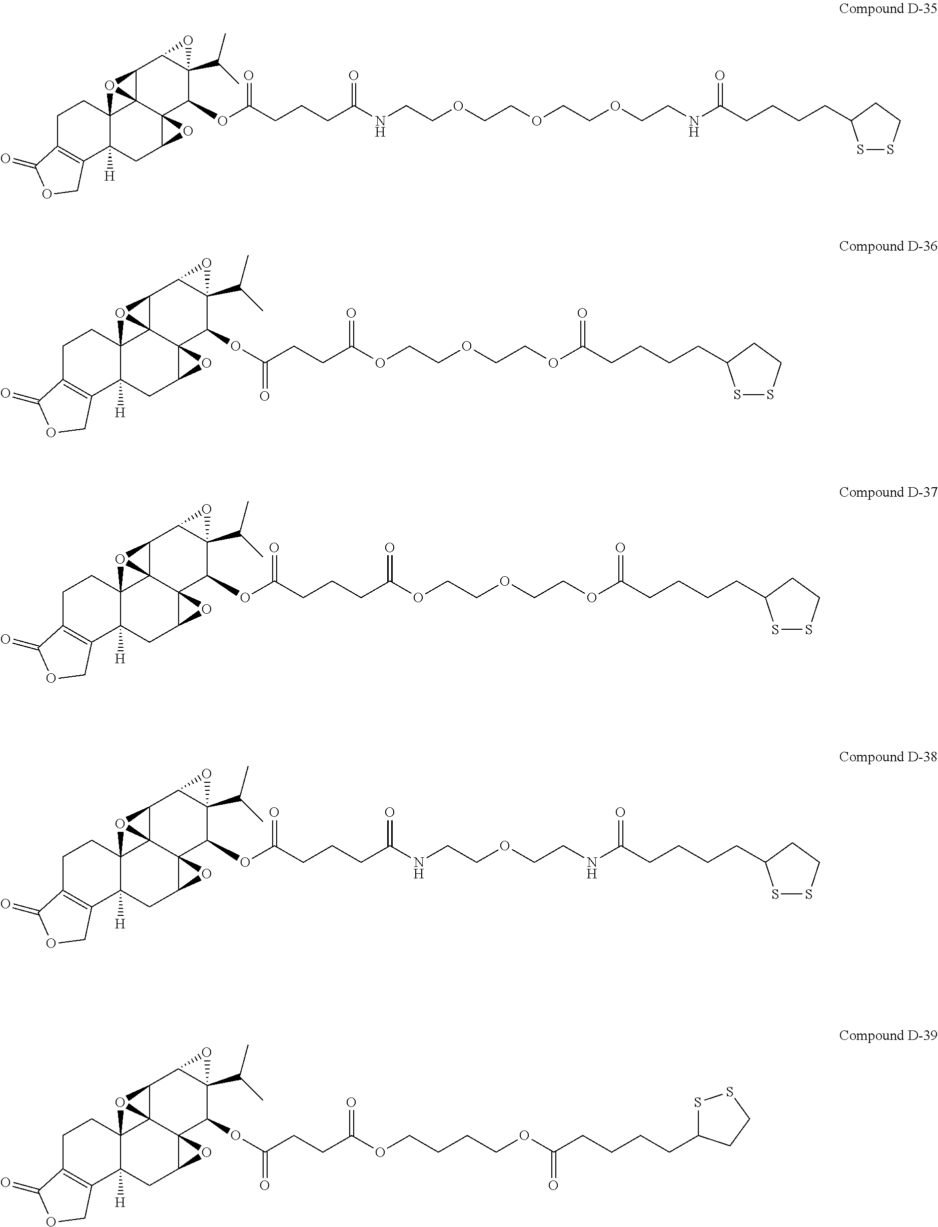

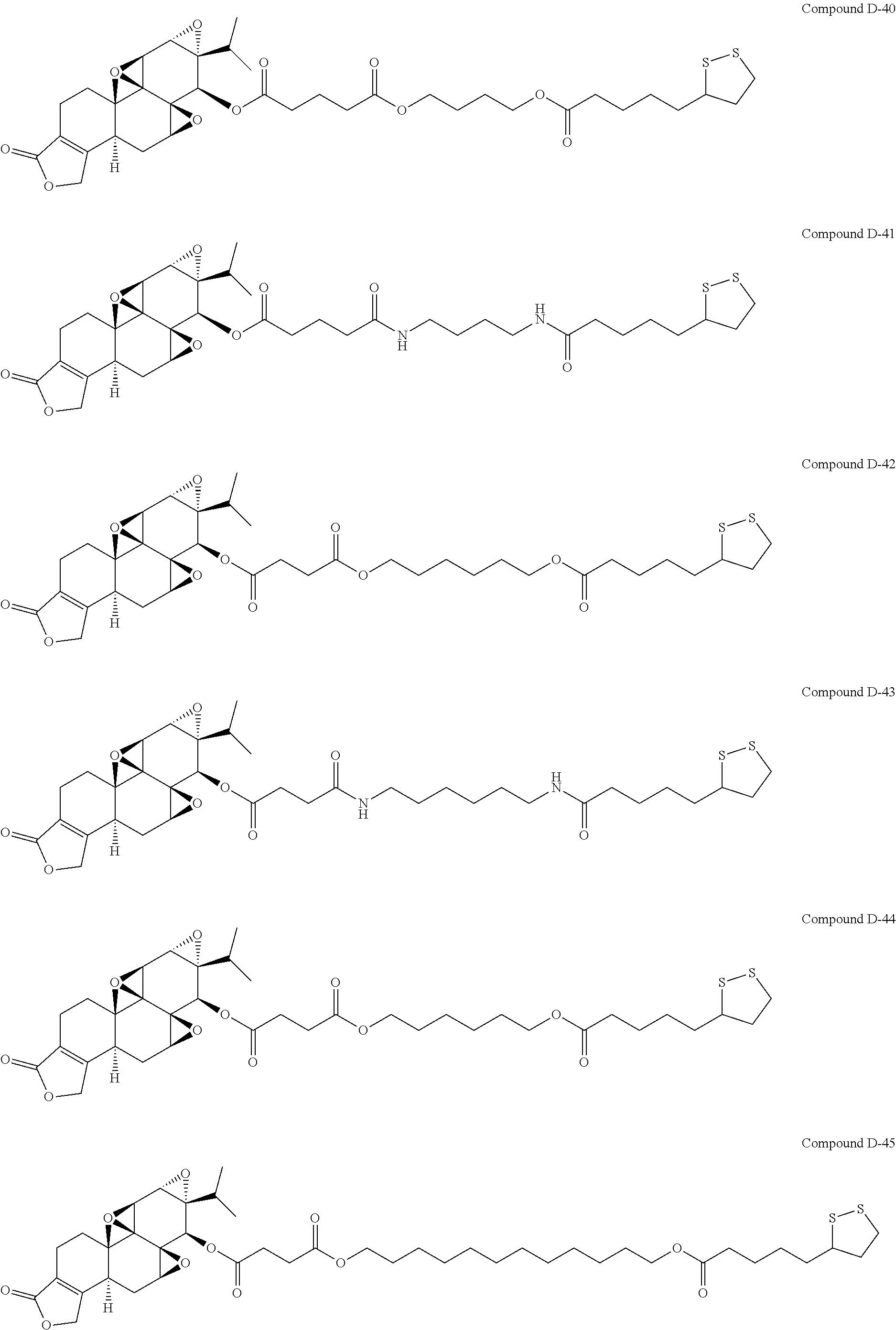

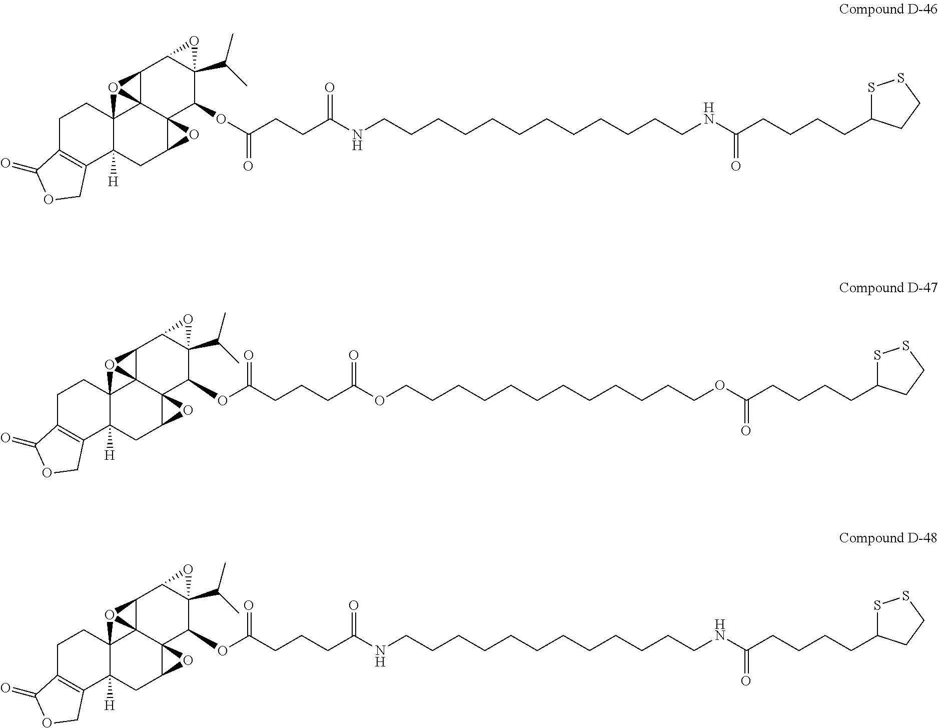

In certain embodiments, the nanospheres are formed with tocopherol. Thus, in certain embodiments, the nanospheres comprise tocopherol.

### TPL Derivatives and Nanospheres

Various embodiments of the present invention use TPLD nanospheres comprising a hydrophobic derivative of a TPL. In one embodiment, the nanosphere comprising TPL of the present invention is capable of releasing the TPLD during a prolonged period of time.

The TPLD nanospheres comprise derivatives of TPL. Hydrophobic TPLD of the present invention can be represented by Formula D-I and/or Formula D-II:

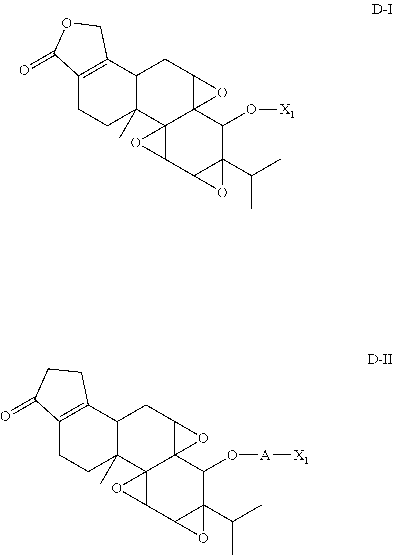

wherein X1 is a antioxidant, an anti-inflammatory or an anticancer agent.

As such, examples of particularly useful hydrophobic derivatives of TPL are represented above.

### Antioxidant, Anti-inflammatory and Anti-Cancer Derivatives and Nanospheres

Various embodiments of the present invention use antioxidant and TPLD nanospheres. In one embodiment, antioxidant and TPLD nanospheres are capable of releasing the TPLDs during a prolonged period of time.

Hydrophobic antioxidant and anti-inflammatory derivatives of a TPL of the present invention can be represented by Formula D-III:

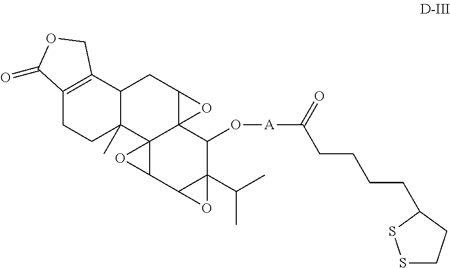

wherein A is as described above.

In some embodiment, the TPL derivatives of the present invention can be represented by Formula D-V:

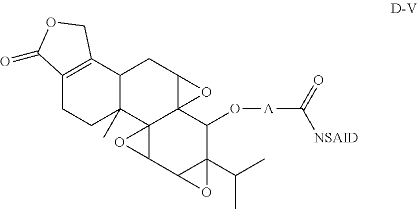

wherein A is as described above.

In some embodiments, the TPL derivatives of the present invention can be represented by Formula D-X:

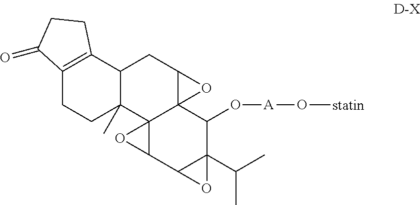

wherein A is as described above.

In some embodiments, the TPL derivatives of the present invention can be represented by Formula D-XI:

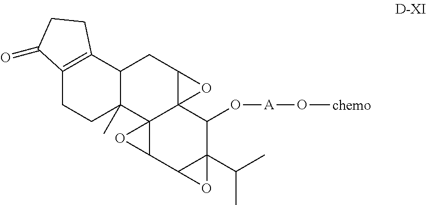

wherein A is as described above.

In some embodiments, the TPL derivatives of the present invention can be represented by Formula D-IV:

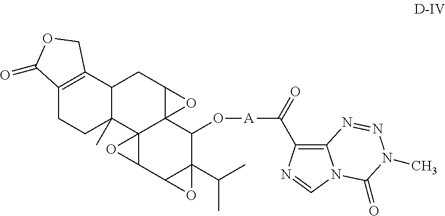

wherein A is as described above.

In some embodiments, the TPL derivatives of the present invention can be represented by Formula D-XII:

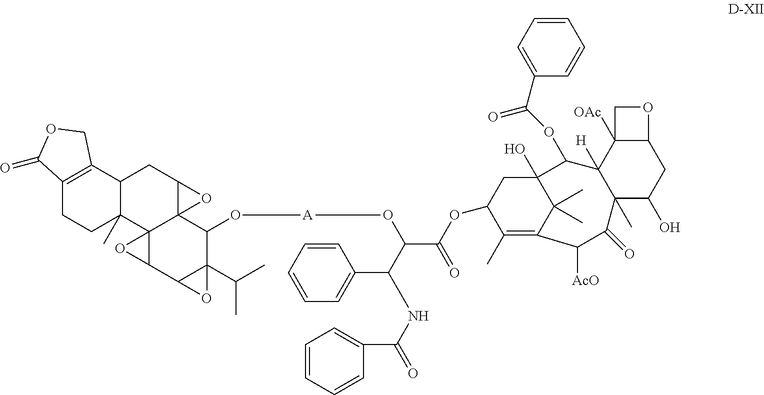

wherein A is as described above.

In some embodiments, the TPL derivatives of the present invention can be represented by Formula D-XIII:

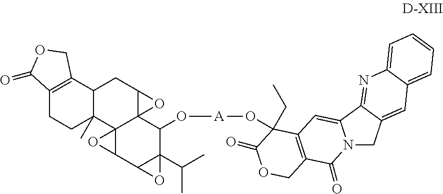

wherein A is as described above.

Examples of particularly useful hydrophobic antioxidant and TPL derivatives are described above.

### Therapeutic Agents

In various embodiments, the therapeutic agent is a triptolide derivative of the invention. It is appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It is appreciated that the therapies employed can achieve a desired effect for the same disorder (for example, an inventive compound can be administered concurrently with another chemotherapeutic agent), or they can achieve different effects (e.g., control of an adverse effects).

For example, other therapies, imaging agents, and/or cancer therapeutic agents that can be used in combination with the inventive compounds of the present invention for imaging, targeting, detecting and/or treating tumors including, radiation therapy, surgery, gemcitabine, cisplastin, paclitaxel, carboplatin, bortezomib, AMG479, vorinostat, rituximab, temozolomide, rapamycin, ABT-737, PI-103; alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE®. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb.®.); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

In some embodiments, the therapeutic agent is a statin. Exemplary statins include, but are not limited to atorvastatin, cerivastatin, fluvastatin, lovastatin, lactones of lovastatin, mevastatin, pitavastatin, pravastatin, rosuvastatin, velostatin, rivastatin, itavastatin, simvastatin, and lactones thereof.

In some embodiments, the therapeutic agent is an antioxidant.

In some embodiments, the therapeutic agent is erythropoietin, peptide, antisense nucleic acid, DNA, RNA, or protein.

In certain embodiments, the pharmaceutical compositions of the present invention further comprise one or more additional therapeutically active ingredients (e.g., chemotherapeutic and/or palliative). For purposes of the invention, the term “palliative” refer, to treatment that is focused on the relief of symptoms of a disease and/or side effects of a therapeutic regimen, but is not curative.

The radioactive salts of the present invention can be especially useful in the treatment of cancer, although other patient treatments are also within the scope of the present invention.

In embodiments which involve salts of compounds of the invention containing radiolabelled phosphorous, and especially, the implantation of the radioactive salts in a tumor in vivo can provide desirable exposure of the tumor to radiation while minimizing the exposure to radiation of nearby, normal tissue. It is contemplated that a wide variety of cancers, especially solid tumor cancers, can be treated using the radioactive salts of the present invention. Examples of such solid tumor cancers include, for example, cancers of the head, such as brain cancer, and cancers of the neck, endometrium, liver, breast, ovaries, cervix and prostate. Embodiments of the invention which involve radioactive salts compounds of the invention, can be particularly suitable for use in the treatment of cancer. The radioactive salts of the present invention can be administered to the patient in a variety of forms, depending on the particular route of administration, the particular salt and/or isotope involved, the particular cancer being treated, and the like. In the case of brachytherapy, the radioactive salts can be administered using techniques which are well known to those skilled in the art, including, for example, surgical implantation. In the case of the administration of radioactive salts in the form, for example, of an aqueous composition or suspension (discussed more fully hereafter), the aqueous composition or suspension can be administered by being injected at the desired site. In addition, the radioactive salts of the present invention can be administered in the form of a radiopharmaceutical matrix (discussed more fully hereafter), also by injection or surgical implantation at the desired site. The particular technique employed for administering the matrix can depend, for example, on the shape and dimensions of the involved matrix. In some embodiments, the radioactive salt is introduced substantially homogeneously in a tumor to minimize the occurrence in the tumor of cold (untreated) areas. In certain embodiments, the radioactive salt is administered in combination with a pharmaceutically acceptable carrier. A wide variety of pharmaceutically acceptable carriers are available and can be combined with the present radioactive salts. Such carriers would be apparent to one skilled in the art, based on the present disclosure. In some embodiments, any material used as a carrier is biocompatible.

Nanospheres Prepared from Mixture of the Inventive TPL Derivatives and Polymers and/or Oils

Various embodiments of the present invention also provide for a nanosphere comprising a TPL derivative and a polymer and/or oily product. The TPL derivatives can be ones as described above. Examples of polymers include, but not limited to, polyanhydrides, polyesters, polyorthoesters, polyesteramides, polyacetals, polyketals, polycarbonates, polyphosphoesters, polyphosphazene, polyvinylpyrrolidone, polydioxanones, poly(malic acid), poly(amino acids), polymers of N-2-(hydroxypropyl)methacrylamide (HPMA), polymers of N-isopropyl acrylamide (NIPAAm), polyglycolide, polylactide, copolymers of glycolide and lactide (e.g., poly(lactide-co-glycolide), and blends thereof. Examples of oily products include, but not limited to, vegetable oils, mineral oils, vitamins, esters of carboxylic acids and combinations thereof.

TPLD Nanospheres Combined with Antioxidant Nanospheres

Various embodiments of the present invention also provide for a composition comprising Antioxidant nanospheres in combination with TPLD nanospheres or Antioxidant and TPLD nanospheres (“TPLD nanosphere/antioxidant nanosphere composition”). The TPLD nanospheres and the Antioxidant and TPLD nanospheres can be ones as described above. The antioxidant nanospheres can be ones as described in International Application No. PCT/US08/88541, incorporated herein by references as though fully set forth.

Briefly, the antioxidant nanospheres comprise an antioxidant molecule represented by the Formula A-IV:

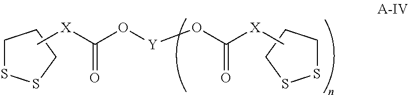

wherein X is selected from the group consisting of a substituted, unsubstituted, branched or unbranched chain of carbon atoms and can optionally contain a heteroatom; Y is selected from the group consisting of branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic groups; and n is an integer and is at least one.

In some embodiments, X is selected from the group consisting of branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl. In some embodiments Y is selected from the group consisting of branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic groups. In various embodiments, Y is a moiety that is formed by esterification of at least two free esterifiable hydroxyl groups on a polyol. In one embodiment, the [1,2]-dithiolane moieties are from α-lipoic acid, and the antioxidants molecules are generally represented by the formula A-V:

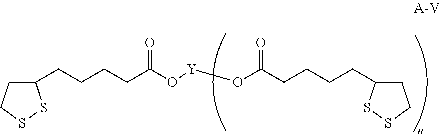

In this embodiment, at least two α-lipoic acids are linked to a polyol via ester bonds. The polyols can be ones known in the art and as described above.

### TPLD/Antioxidant Nanosphere Combination

Various embodiments of the present invention also provide for a nanosphere comprising molecules selected from Formula D-I and/or Formula D-II as described above, and molecules selected from Formula A-IV or A-V as described above (“TPLD/antioxidant nanosphere combination”).

### Amphiphilic Spacer

A hydrophilic or hydrophobic spacer used in the present disclosure is a molecule that comprises hydrophilic or hydrophobic parts in one molecule, and further comprises chemically active functional group on one end or both ends which can be used as a carrier for a therapeutic agent, diagnostic agent, or another spacer by conjugating it with the therapeutic agent, diagnostic agent, or another spacer molecule.

An amphiphilic spacer used in the present disclosure is a molecule that comprises both hydrophilic and hydrophobic parts in one molecule, and the hydrophilic part can further comprise a chemically active functional group which can be used as a carrier for a therapeutic or diagnostic agent by conjugating it with the therapeutic agent or diagnostic agent. In various embodiments, the chemically active functional group can be selected from the group consisting of thiol, amine, carboxylic acid, carboxylic acid NHS ester, maleimide, hydrazine, ketone, and aldehyde. An amphiphilic spacer used in the present disclosure also can be made by conjugating a hydrophilic spacer with a hydrophobic spacer. The end of the hydrophilic part further comprises chemically active functional group which can be used as a carrier for a therapeutic or diagnostic agent by conjugating it with the therapeutic agent or diagnostic agent.

In various embodiments, the amphiphilic spacer comprises a hydrophobic part and hydrophilic part. In various embodiments, the hydrophobic part of amphiphilic spacer is selected from the group consisting of branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic groups, and combinations thereof.

In various embodiments, the hydrophilic part of amphiphilic spacer comprises a molecule selected from the group consisting of heteroatom-containing branched and unbranched alkenyl, heteroatom-containing branched and unbranched alkynyl, aryl, cyclic aliphatic, cyclic aromatic, heterocyclic, and aromatic heterocyclic groups, and a chemically active group selected from the group consisting of thiol, amine, carboxylic acid, carboxylic acid NHS ester, maleimide, hydrazine, ketone, aledehyde, and combinations thereof.

In various embodiments, the amphiphilic spacer comprises an alkylthiol. In various embodiments, the amphiphilic spacer is an alkylthiol. In some embodiments, the alkylthiol is C2-4alkylthiol. In some embodiments, the alkylthiol is C2-4alkylthiol. In some embodiments, the alkylthiol is C4-6alkylthiol. In some embodiments, the alkylthiol is C6-8alkylthiol. In some embodiments, the alkylthiol is C8-10alkylthiol. In some embodiments, the alkylthiol is C10-12alkylthiol. In some embodiments, the alkylthiol is C12-14alkylthiol. In some embodiments, the alkylthiol is C14-18alkylthiol. In some embodiments, the alkylthiol is C18-20alkylthiol. In some embodiments, the alkylthiol is C10-18alkylthiol. In some embodiments, the alkylthiol is C22-24alkylthiol. In some embodiments, the alkylthiol is C24-30alkylthiol. In various embodiments, the alkylthiol is a straight chain alkylthiol.

In various embodiments, the amphiphilic spacer is selected from a C10, C11, C12, C13, C14, C15, C16, C17, C18, C19, C20, C21, C22, C23, C24, C25, C26, C27, C28, C29, C30, C31, C32, C34, C35, C36, C37, C38, C39 and C40 straight chain alkylthiol. In some embodiments, the amphiphilic spacer is 1-octadecanethiol.

In various embodiments, the amphiphilic spacer comprises an alkylamine. In various embodiments, the amphiphilic spacer is an alkylamine. In some embodiments, the alkylamine is C2-4alkylamine. In some embodiments, the alkylamine is C2-4alkylamine. In some embodiments, the alkylamine is C4-6alkylamine. In some embodiments, the alkylamine is C6-8alkylamine. In some embodiments, the alkylamine is C8-10alkylamine. In some embodiments, the alkylamine is C10-12alkylamine. In some embodiments, the alkylamine is C12-14alkylamine. In some embodiments, the alkylamine is C14-18alkylamine. In some embodiments, the alkylamine is C18-20alkylamine. In some embodiments, the alkylamine is C10-18alkylamine. In some embodiments, the alkylamine is C22-24alkylamine. In some embodiments, the alkylamine is C24-30alkylamine. In some embodiments, the alkylamine is a straight chain alkylamine.

In various embodiments, the amphiphilic spacer is selected from a C10, C11, C12, C13, C14, C15, C16, C17, C18, C19, C20, C21, C22, C23, C24, C25, C26, C27, C28, C29, C30, C31, C32, C34, C35, C36, C37, C38, C39 and C40 straight chain alkylamine.

### Amphiphilic Polymer

In various embodiments, the amphiphilic polymer comprises a polymer backbone, a hydrophilic part of the polymer and a hydrophobic part of the polymer. In various embodiments, the polymer backbone can be from natural polymer, modified natural polymer, synthetic polymer, and combinations thereof.

In some embodiments, the nanosphere further comprises an amphiphilic spacer.

In various embodiments, the polymer backbone is selected from the group consisting of a polyanhydride, polyester, polyorthoester, polyesteramide, polyacetal, polyketal, polycarbonate, polyphosphoester, polyphosphazene, polyvinylpyrrolidone, polydioxanone, poly(malic acid), poly(amino acid), polymer of N-2-(hydroxypropyl)methacrylamide (HPMA), polymer of N-isopropyl acrylamide (NIPAAm), polyglycolide, polylactide, copolymer of glycolide and lactide, and combinations thereof.

In various embodiments, the hydrophobic part of amphiphilic polymer is selected from the group consisting of branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic groups, and combinations thereof.

In various embodiments, the hydrophilic part of amphiphilic polymer comprises a molecule selected from the group consisting of heteroatom-containing branched and unbranched alkenyl, heteroatom-containing branched and unbranched alkynyl, aryl, cyclic aliphatic, cyclic aromatic, heterocyclic, and aromatic heterocyclic groups, and a chemically active group selected from the group consisting of thiol, amine, carboxylic acid, carboxylic acid NHS ester, maleimide, hydrazine, ketone, aledehyde, and combinations thereof.

In various embodiments, the amphiphilic polymer comprises an alkylthiol. In various embodiments, the amphiphilic spacer is an alkylthiol. In some embodiments, the alkylthiol is C2-4alkylthiol. In some embodiments, the alkylthiol is C2-4alkylthiol. In some embodiments, the alkylthiol is C4-6alkylthiol. In some embodiments, the alkylthiol is C6-8alkylthiol. In some embodiments, the alkylthiol is C8-10alkylthiol. In some embodiments, the alkylthiol is C10-12alkylthiol. In some embodiments, the alkylthiol is C12-14alkylthiol. In some embodiments, the alkylthiol is C14-18alkylthiol. In some embodiments, the alkylthiol is C18-20alkylthiol. In some embodiments, the alkylthiol is C10-18alkylthiol. In some embodiments, the alkylthiol is C22-24alkylthiol. In some embodiments, the alkylthiol is C24-30alkylthiol. In some embodiments, the alkylthiol is a straight chain alkylthiol. In various embodiments, the amphiphilic polymer is selected from a C2, C3, C4, C6, C7, C8, C9, C10, C11, C12, C13, C14, C15, C16, C17, C18, C19, C20, C21, C22, C23, C24, C25, C26, C27, C28, C29, C30, C31, C32, C34, C35, C36, C37, C38, C39 and C40 straight chain alkylthiol.

In various embodiments, the amphiphilic polymer comprises an alkylamine. In various embodiments, the amphiphilic polymer is an alkylamine. In some embodiments, the alkylamine is C2-4alkylamine. In some embodiments, the alkylamine is C2-4alkylamine. In some embodiments, the alkylamine is C4-6alkylamine. In some embodiments, the alkylamine is C6-8alkylamine. In some embodiments, the alkylamine is C8-10alkylamine. In some embodiments, the alkylamine is C10-12alkylamine. In some embodiments, the alkylamine is C12-14alkylamine. In some embodiments, the alkylamine is C14-18alkylamine. In some embodiments, the alkylamine is C18-20alkylamine. In some embodiments, the alkylamine is C10-18alkylamine. In some embodiments, the alkylamine is C22-24alkylamine. In some embodiments, the alkylamine is C24-30alkylamine. In some embodiments, the alkylamine is a straight chain alkylamine.

In various embodiments, the amphiphilic polymer is selected from a C2, C3, C4, C6, C7, C8, C9, C10, C11, C12, C13, C14, C15, C16, C17, C18, C19, C20, C21, C22, C23, C24, C25, C26, C27, C28, C29, C30, C31, C32, C34, C35, C36, C37, C38, C39 and C40 straight chain alkylamine

Accordingly, in various embodiments, the nanospheres used in the present invention comprise a hydrophobic TPL derivative, tocopherol and a therapeutic agent or a diagnostic agent conjugated to a hydrophilic, hydrophobic, or amphiphilic spacer.

In certain embodiments, the nanospheres comprise a hydrophobic TPL derivative, tocopherol and an antioxidant α-lipoic acid-containing hydrophobic compound and therapeutic agent or a diagnostic agent conjugated to a hydrophilic, hydrophobic, or amphiphilic spacer.

In certain embodiments, the nanospheres comprise tocopherol and a hydrophobic antioxidant and anti-inflammatory derivative of a TPL and a therapeutic agent or a diagnostic agent conjugated to a hydrophilic, hydrophobic, or amphiphilic spacer.

In various embodiments, the nanospheres comprise a hydrophobic TPL derivative, tocopherol and a therapeutic agent or a diagnostic agent conjugated to an amphiphilic polymer.

In certain embodiments, the nanospheres comprise a hydrophobic TPL derivative, tocopherol and an antioxidant α-lipoic acid-containing hydrophobic compound and therapeutic agent or a diagnostic agent conjugated to an amphiphilic polymer.

In certain embodiments, the nanospheres comprise tocopherol and a hydrophobic antioxidant and anti-inflammatory derivative of a TPL and a therapeutic agent or a diagnostic agent conjugated to an amphiphilic polymer.

### Labeling

Various embodiments provide for methods of imaging and diagnosing cancer. The method can comprise providing a cancer-targeted nanosphere of the present invention

wherein the nanosphere further comprises a detectable label; administering the nanosphere to a subject in need thereof; and imaging the subject to detect the cancer. In some embodiments, detectable label is conjugated to a hydrophilic spacer, a hydrophobic spacer, an amphiphilic spacer, or an amphiphilic polymer.

As used herein, the term “detectable label” refers to a composition capable of producing a detectable signal indicative of the presence of a target. Generally, a detectable label is any composition detectable by spectroscopic, photochemical, biochemical, immunochemical, electrical, optical or chemical means. Suitable labels include fluorescent molecules, radioisotopes, nucleotide chromophores, enzymes, substrates, chemiluminescent moieties, magnetic particles, bioluminescent moieties, and the like. As such, a label is any composition detectable by spectroscopic, photochemical, biochemical, immunochemical, electrical, optical or chemical means needed for the methods and devices described herein. For example, the peptides can be labeled with a detectable tag which can be detected using an antibody specific to the label.

In some embodiments, the detectable label can be an imaging agent, diagnostic agent, or contrast agent. As used herein, the term “imaging agent” refers to an element or functional group in a molecule that allows for the detection, imaging, and/or monitoring of the presence and/or progression of a condition(s), pathological disorder(s), and/or disease(s). The imaging agent can be an echogenic substance (either liquid or gas), non-metallic isotope, an optical reporter, a boron neutron absorber, a paramagnetic metal ion, a ferromagnetic metal, a gamma-emitting radioisotope, a positron-emitting radioisotope, or an x-ray absorber. As used herein the term “contrast agent” refers to any molecule that changes the optical properties of tissue or organ containing the molecule. Optical properties that can be changed include, but are not limited to, absorbance, reflectance, fluorescence, birefringence, optical scattering and the like. In some embodiments, the detectable labels also encompass any imaging agent (e.g., but not limited to, a bubble, a liposome, a sphere, a contrast agent, or any detectable label described herein) that can facilitate imaging or visualization of a tissue or an organ in a subject, e.g., for diagnosis of an infection. In some embodiments, the imaging agent can be an antibody, or an epitope binding fragment thereof, that binds a protein expressed or overexpressed in cancer.

Suitable optical reporters include, but are not limited to, fluorescent reporters and chemiluminescent groups. A wide variety of fluorescent reporter dyes are known in the art. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound.

Exemplary fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein (pH 10); 5-Carboxytetramethylrhodamine (5-TAMRA); 5-FAM (5-Carboxyfluorescein); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Anilin Blue; Anthrocyl stearate; APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); BG-647; Bimane; Bisbenzamide; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CFDA; CFP—Cyan Fluorescent Protein; Chlorophyll; Chromomycin A; Chromomycin A; CMFDA; Coelenterazine; Coelenterazine cp; Coelenterazine f; Coelenterazine fcp; Coelenterazine h; Coelenterazine hcp; Coelenterazine ip; Coelenterazine O; Coumarin Phalloidin; CPM Methylcoumarin; CTC; Cy2™; Cy3.1 8; Cy3.5™; Cy3™; Cy5.1 8; Cy5.5™; Cy5™; Cy7™; Cyan GFP; cyclic AMP Fluorosensor (FiCRhR); d2; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); DIDS; Dihydorhodamine 123 (DHR); DiO (DiOC18(3)); DiR; DiR (DiIC18(7)); Dopamine; DsRed; DTAF; DY-630-NHS; DY-635-NHS; EBFP; ECFP; EGFP; ELF 97; Eosin; Erythrosin; Erythrosin ITC; Ethidium homodimer-1 (EthD-1); Euchrysin; Europium (III) chloride; Europium; EYFP; Fast Blue; FDA; Feulgen (Pararosaniline); FITC; FL-645; Flazo Orange; Fluo-3; Fluo-4; Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura-2, high calcium; Fura-2, low calcium; Genacryl Brilliant Red B; Genacryl Brilliant Yellow 10GF; Genacryl Pink 3G; Genacryl Yellow 5GF; GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indodicarbocyanine (DiD); Indotricarbocyanine (DiR); Intrawhite Cf; JC-1; JO-J0-1; JO-PRO-1; LaserPro; Laurodan; LDS 751; Leucophor PAF; Leucophor SF; Leucophor WS; Lissamine Rhodamine; Lissamine Rhodamine B; LOLO-1; LO-PRO-1; Lucifer Yellow; Mag Green; Magdala Red (Phloxin B); Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant lavin E8G; Oregon Green™; Oregon Green 488-X; Oregon Green™ 488; Oregon Green™ 500; Oregon Green™ 514; Pacific Blue; Pararosaniline (Feulgen); PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26; PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B 540; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycoerythrin (PE); red shifted GFP (rsGFP, S65T); S65A; S65C; S65L; S65T; Sapphire GFP; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SPQ (6-methoxy-N-(3-sulfopropyl)-quinolinium); Stilbene; Sulphorhodamine B can C; Sulphorhodamine G Extra; Tetracycline; Tetramethylrhodamine; Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodamineIsoThioCyanate); True Blue; TruRed; Ultralite; Uranine B; Uvitex SFC; wt GFP; WW 781; XL665; X-Rhodamine; XRITC; Xylene Orange; Y66F; Y66H; Y66W; Yellow GFP; YFP; YO-PRO-1; YO-PRO-3; YOYO-1; and YOYO-3. Many suitable forms of these fluorescent compounds are available and can be used.

Other exemplary detectable labels include luminescent and bioluminescent markers (e.g., biotin, luciferase (e.g., bacterial, firefly, click beetle and the like), luciferin, and aequorin), radiolabels (e.g., 3H, 125I, 35S, 14C, or 32P), enzymes (e.g., galactosidases, glucorinidases, phosphatases (e.g., alkaline phosphatase), peroxidases (e.g., horseradish peroxidase), and cholinesterases), and calorimetric labels such as colloidal gold or colored glass or plastic (e.g., polystyrene, polypropylene, and latex) beads. Patents teaching the use of such labels include U.S. Pat. Nos. 3,817,837, 3,850,752, 3,939,350, 3,996,345, 4,277,437, 4,275,149, and 4,366,241, each of which is incorporated herein by reference.

Suitable echogenic gases include, but are not limited to, a sulfur hexafluoride or perfluorocarbon gas, such as perfluoromethane, perfluoroethane, perfluoropropane, perfluorobutane, perfluorocyclobutane, perfluropentane, or perfluorohexane. Suitable non-metallic isotopes include, but are not limited to, ¹¹C, ¹⁴C, ¹³N, ¹⁸F, ¹²³I, ¹²⁴I, and ¹²⁵I. Suitable radioisotopes include, but are not limited to, ⁹⁹mTc, ⁹⁵Tc, ¹¹¹In, ⁶²Cu, ⁶⁴Cu, Ga, ⁶⁸Ga, and ¹⁵³Gd. Suitable paramagnetic metal ions include, but are not limited to, Gd(III), Dy(III), Fe(III), and Mn(II). Suitable X-ray absorbers include, but are not limited to, Re, Sm, Ho, Lu, Pm, Y, Bi, Pd, Gd, La, Au, Au, Yb, Dy, Cu, Rh, Ag, and Ir.

In some embodiments, the radionuclide can be bound to a chelating agent. Suitable radionuclides for direct conjugation include, without limitation, ¹⁸F, ¹²⁴I, ¹²⁵I, ¹³¹I, and mixtures thereof. Suitable radionuclides for use with a chelating agent include, without limitation, ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In ¹¹⁷msn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and mixtures thereof. Suitable chelating agents include, but are not limited to, DOTA, BAD, TETA, DTPA, EDTA, NTA, HDTA, their phosphonate analogs, and mixtures thereof.

Means of detecting such labels are well known to those of skill in the art. Thus, for example, radiolabels can be detected using photographic film or scintillation counters, fluorescent markers can be detected using a photo-detector to detect emitted light. Enzymatic labels are typically detected by providing the enzyme with an enzyme substrate and detecting the reaction product produced by the action of the enzyme on the enzyme substrate, and calorimetric labels can be detected by visualizing the colored label. Exemplary methods for in vivo detection or imaging of detectable labels include, but are not limited to, radiography, magnetic resonance imaging (MRI), Positron emission tomography (PET), Single-photon emission computed tomography (SPECT, or less commonly, SPET), Scintigraphy, ultrasound, CAT scan, photoacoustic imaging, thermography, linear tomography, poly tomography, zonography, orthopantomography (OPT or OPG), and computed Tomography (CT) or Computed Axial Tomography (CAT scan).

In some embodiments, the detectable label is a fluorophore or a quantum dot. Without wishing to be bound by a theory, using a fluorescent reagent can reduce signal-to-noise in the imaging/readout, thus maintaining sensitivity.

In various embodiments, the imaging and/or diagnostic agents can include, but are not limited to fluorescent dyes, radiolabels, and antibodies against proteins overexpressed in cancer.

Exemplary fluorescent labeling reagents include, but are not limited to, Hydroxycoumarin, Succinimidyl ester, Aminocoumarin, Methoxycoumarin, Cascade Blue, Hydrazide, Pacific Blue, Maleimide, Pacific Orange, Lucifer yellow, NBD, NBD-X, R-Phycoerythrin (PE), a PE-Cy5 conjugate (Cychrome, R670, Tri-Color, Quantum Red), a PE-Cy7 conjugate, Red 613, PE-Texas Red, PerCP, Peridinin chlorphyll protein, TruRed (PerCP-Cy5.5 conjugate), FluorX, Fluoresceinisothyocyanate (FITC), BODIPY-FL, TRITC, X-Rhodamine (XRITC), Lissamine Rhodamine B, Texas Red, Allophycocyanin (APC), an APC-Cy7 conjugate, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, Alexa Fluor 790, Cy2, Cy3, Cy3B, Cy3.5, Cy5, Cy5.5 or Cy7.

Examples of radiolabels include but are not limited to ²H, ¹³C, ¹⁵N, iodophenylalanine, Tc99m, iodination.

One aspect of the invention relates to the fluorescent labeling of the nanospheres comprising TPLD. In some embodiments, a compound of Formula D-I and/or Formula D-II is combined with an antioxidant and a thiol. In some embodiments the thiol is a C18-C20 thiol. In some embodiments the thiol is C18-C22 thiol. In some embodiments, the thiol is C18-C25 thiol. In some embodiments, the thiol is C18-C28 thiol. In some embodiments, the thiol is C18-C32 thiol. In some embodiments, the thiol is C20-C25 thiol.

In some embodiments the compound of Formula D-I and/or D-II is combined with an antioxidant and a thiol, and further with a fluorescent tag. In some embodiments, the fluorescent tag comprises a maleimide functionality. In some embodiments, the fluorescent tag is a cyanine. In some embodiments, the cyanine is Cy3 or Cy5. In some embodiments, the fluorescent tag is an Alexa fluor dye, FluoProbes dye, Sulfo Cy dye or Seta dye. In some embodiments, the fluorescent is another fluorophore.

### Methods of Using the Nanospheres

Additional embodiments of the present invention provide for methods of using the TPLD nanospheres of the present invention, the antioxidant and TPLD nanospheres of the present invention, the TPLD nanosphere/antioxidant nanosphere composition of the present invention, or TPLD/antioxidant nanosphere combination of the present invention.

In some embodiments, methods of deliverying a nanosphere to a tumor or cancer tissue in a subject, comprising: administering a therapeutically effective amount of a nanosphere to the subject, wherein the nanosphere comprises a compound selected from Formula D-I, Formula D-II, and any combinations thereof.

In some embodiments, the nanosphere further comprises an antioxidant. In certain embodiments, the antioxidant is tocopherol or a derivative thereof.

In some embodiments, the nanosphere further comprises a compound of Formula A-IV or Formula A-V.

In some embodiments, the nanosphere further comprises an amphiphilic spacer.

In some embodiments, the amphiphilic spacer comprises a chemically active group selected from the group consisting of thiol, amine, carboxylic acid, carboxylic acid NHS ester, maleimide, hydrazine, ketone, aledehyde, and combinations thereof

In some embodiments, the amphiphilic spacer is an alkylthiol or an alkylamine. In some embodiments, the amphiphilic spacer is 1-octadecanethiol.

In some embodiments, the nanosphere further comprises a polymer. In some embodiments, the polymer is poly(lactide-co-glycolide) (PLGA).

In some embodiments, the nanosphere further comprises a therapeutic agent.

In some embodiments, the therapeutic agent is selected from the group consisting of: chemotherapeutic agents, statins, nonsteroidal anti-inflammatory drugs (NSAID), erythropoietin, peptides, antisense nucleic acid, DNA, RNA, protein, and combinations thereof.

In some embodiments, the therapeutic agent is delivered to the tumor or cancer tissue.

In some embodiments, the delivery of the therapeutic agent to the tumor or cancer tissue treats cancer.

In some embodiments, the therapeutic agent is conjugated to a hydrophilic spacer, a hydrophobic spacer, an amphiphilic spacer, or an amphiphilic polymer.

In some embodiments, the nanosphere further comprises an imaging agent. In some embodiments, the imaging agent is conjugated to a hydrophilic spacer, a hydrophobic spacer, an amphiphilic spacer, or an amphiphilic polymer.

In some embodiments, the imaging agent is selected from the group consisting of fluorescent molecules, radioisotopes, nucleotide chromophores, enzymes, substrates, chemiluminescent moieties, magnetic particles, bioluminescent moieties, antibody against a protein expressed or overexpressed in cancer, and combinations thereof.

In some embodiments, delivering the nanosphere to the tumor or cancer tissue treats cancer.

In some embodiments nanospheres can be used for treating inflammation or diseases or disease conditions that are caused by or related to inflammation in subjects in need thereof. The method comprises providing a composition comprising the TPLD nanospheres of the present invention, the antioxidant and TPLD nanospheres of the present invention, the TPLD nanosphere/antioxidant nanosphere composition of the present invention, or TPLD/antioxidant nanosphere combination of the present invention, and administering a therapeutically effective amount of the composition to the subject in need thereof.

In some particular embodiments, the TPLD nanospheres of the present invention, the antioxidant and TPLD nanospheres of the present invention, the TPLD nanosphere/antioxidant nanosphere composition of the present invention, or TPLD/antioxidant nanosphere combination of the present invention are used to treat cancer in a subject in need thereof. The method comprises providing a composition comprising the TPLD nanospheres of the present invention, the antioxidant and TPLD nanospheres of the present invention, the TPLD nanosphere/antioxidant nanosphere composition of the present invention, or TPLD/antioxidant nanosphere combination of the present invention and administering a therapeutically effective amount of the composition to the subject.

In some particular embodiments, the nanospheres comprising TPLD of the present are used to treat cancer in a subject in need thereof. The method comprises providing a composition comprising nanospheres comprising TPLD of the present invention, and administering a therapeutically effective amount of the composition to the subject.

In some embodiments, the method of treating cancer comprises providing a nanosphere of the present invention wherein the nanosphere further comprises a therapeutic agent conjugated to a hydrophilic spacer, a hydrophobic spacer, an amphiphilic spacer, or an amphiphilic polymer; and administering the nanosphere to a subject in need thereof.

In some embodiments, the method of treating cancer comprises providing a nanosphere of the present invention wherein a therapeutic agent is not conjugated to a hydrophilic spacer, a hydrophobic spacer, an amphiphilic spacer, or an amphiphilic polymer; and administering the nanosphere to a subject in need thereof.

In another embodiment, the TPLD nanospheres of the present invention, the antioxidant and TPLD nanospheres of the present invention, the TPLD nanosphere/antioxidant nanosphere composition of the present invention, or TPLD/antioxidant nanosphere combination of the present invention can be used as a carrier of a therapeutic agent. In one embodiment, the therapeutic agent is an additional TPLD that is useful for cancer treatment. In another embodiment, the therapeutic agent is an additional agent that is useful for cancer treatment. Accordingly, the present invention provides for a composition comprising the TPLD nanospheres of the present invention, the antioxidant and TPLD nanospheres of the present invention, the TPLD nanosphere/antioxidant nanosphere composition of the present invention, or TPLD/antioxidant nanosphere combination of the present invention and a therapeutic agent.

In another embodiment, the TPLD nanospheres of the present invention, the antioxidant and TPLD nanospheres of the present invention, the TPLD nanosphere/antioxidant nanosphere composition of the present invention, or TPLD/antioxidant nanosphere combination of the present invention can also be used as pharmaceutical and/or drug delivery vehicles to deliver small molecules, peptides, oligonucleotides, polynucleotides, proteins, antigens, chemotherapeutics, antisense nucleic acid molecules and the like, to tissue, organ, cell, etc.

### Methods of Preparing the Nanospheres

In another embodiment, the present invention provides for a method of preparing TPLD nanospheres comprising a TPL derivative of the present invention. The method comprises providing a TPL derivative of formula D-I or D-II and processing the TPL derivative in a spontaneous emulsification process.

In another embodiment, the present invention provides for a method of preparing the TPLD/antioxidant nanosphere combination of the present invention. The antioxidant nanosphere can be a molecule as described by International Application No. PCT/US08/88541, which is incorporated herein by reference in its entirety as though fully set forth (e.g., formulas A-IV and A-V). The method comprises providing a TPL derivative of formula D-I or D-II and an antioxidant molecule of formula A-IV or A-V and processing the TPL derivative and antioxidant molecule in a spontaneous emulsification process. In another embodiment the method comprises providing molecules of Formula D-I or D-II and an antioxidant molecule of formula A-IV or A-V and processing the molecules of Formula D-I or D-II and antioxidant molecule in a spontaneous emulsification process

In another embodiment, the present invention provides for a method of preparing the antioxidant and TPLD nanospheres. The method comprises providing a molecule of formula D-I or D-II and processing the molecule in a spontaneous emulsification process.

### Pharmaceutical Compositions

In various embodiments, the present invention provides pharmaceutical compositions including a pharmaceutically acceptable excipient along with a therapeutically effective amount of the nanospheres of the present invention. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

In various embodiments, the pharmaceutical compositions according to the invention can be formulated for delivery via any route of administration. “Route of administration” can refer to any administration pathway known in the art, including but not limited to aerosol, nasal, oral, transmucosal, transdermal, parenteral, enteral, or ocular. “Transdermal” administration can be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions can be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions can be in the form of solutions or suspensions for infusion or for injection. Via the topical route, the pharmaceutical compositions based on compounds according to the invention can be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. Via the ocular route, they can be in the form of eye drops.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier can be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it can come in contact, meaning that it must not carry a risk of toxicity, irritation, allergic response, immunogenicity, or any other complication that excessively outweighs its therapeutic benefits.

Pharmaceutically acceptable carriers include any and all solvents, dispersion media, coatings, antibacterial and antifungal agents, isotonic and absorption delaying agents, and the like which are compatible with the activity of the active agent and are physiologically acceptable to the subject. Some examples of materials which can serve as pharmaceutically-acceptable carriers include: (i) sugars, such as lactose, glucose and sucrose; (ii) starches, such as corn starch and potato starch; (iii) cellulose, and its derivatives, such as sodium carboxymethyl cellulose, methylcellulose, ethyl cellulose, microcrystalline cellulose and cellulose acetate; (iv) powdered tragacanth; (v) malt; (vi) gelatin; (vii) lubricating agents, such as magnesium stearate, sodium lauryl sulfate and talc; (viii) excipients, such as cocoa butter and suppository waxes; (ix) oils, such as peanut oil, cottonseed oil, safflower oil, sesame oil, olive oil, corn oil and soybean oil; (x) glycols, such as propylene glycol; (xi) polyols, such as glycerin, sorbitol, mannitol and polyethylene glycol (PEG); (xii) esters, such as ethyl oleate and ethyl laurate; (xiii) agar; (xiv) buffering agents, such as magnesium hydroxide and aluminum hydroxide; (xv) alginic acid; (xvi) pyrogen-free water; (xvii) isotonic saline; (xviii) Ringer's solution; (xix) ethyl alcohol; (xx) pH buffered solutions; (xxi) polyesters, polycarbonates and/or polyanhydrides; (xxii) bulking agents, such as polypeptides and amino acids (xxiii) serum component, such as serum albumin, HDL and LDL; (xxiv) C2-C12 alcohols, such as ethanol; and (xxv) other non-toxic compatible substances employed in pharmaceutical formulations.

The pharmaceutical compositions according to the invention can also be encapsulated, tableted or prepared in an emulsion or syrup for oral administration. Pharmaceutically acceptable solid or liquid carriers can be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier can also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation can be administered directly p.o. or filled into a soft gelatin capsule.

The pharmaceutical compositions according to the invention can be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

### Dosage

Typical dosages of an effective amount of the antioxidant derivatives of the composition of the invention can be in the ranges recommended by the manufacturer where known therapeutic compounds are used, and also as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on the in vitro responsiveness of the relevant primary cultured cells or histocultured tissue sample, such as biopsied malignant tumors, or the responses observed in the appropriate animal models, as previously described.

### Kits

The present invention is also directed to a kit to deliver a nanosphere of the present invention to a tumor or cancer and a kit to treat cancer. The kit is an assemblage of materials or components, including at least one of the inventive compositions. Thus, in some embodiments the kit contains a composition including the nanospheres of the present invention as described above.

The exact nature of the components configured in the inventive kit depends on its intended purpose. For example, some embodiments are configured for the purpose of delivering a nanosphere of the present invention to a tumor or cancer tissue, and other embodiments are configured for the purpose of treating cancer. In some embodiments, the kit is configured particularly for the purpose of delivering to or treating mammalian subjects. In another embodiment, the kit is configured particularly for the purpose of deliverying to or treating human subjects. In further embodiments, the kit is configured for veterinary applications, treating subjects such as, but not limited to, farm animals, domestic animals, and laboratory animals.

Instructions for use can be included in the kit. “Instructions for use” typically include a tangible expression describing the technique to be employed in using the components of the kit to effect a desired outcome, such as delivery a nanosphere of the present invention to tumor or cancer tissue or to treat cancer. Optionally, the kit also contains other useful components, such as, diluents, buffers, pharmaceutically acceptable carriers, syringes, catheters, applicators, pipetting or measuring tools, or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a glass vial used to contain suitable quantities of an inventive nanospheres comprising a therapeutic agent or an imaging agent optionally conjugated to a hydrophilic spacer, a hydrophobic spacer, an amphiphilic spacer, or an amphiphilic polymer. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1

### Ethics Statement

All experiments were approved by the Institutional Animal Care and Use Committee (IACUC) at the Cedars-Sinai Medical Center (protocol #2620). All efforts were made to minimize suffering through the use of anesthesia, analgesia, and post-injury care and monitoring.

### Triptolide Nanoprodrug

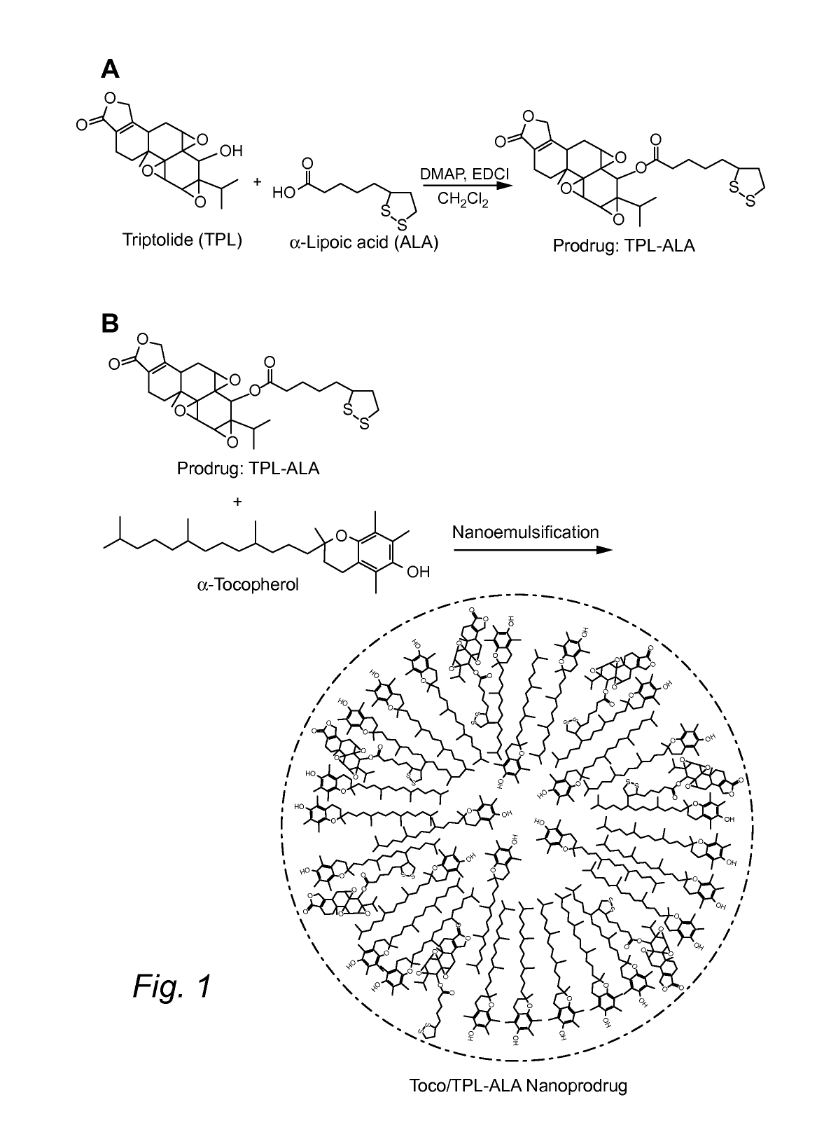

In order to obtain a hydrophobic, biodegradable prodrug, triptolide prodrug from triptolide and α-lipoic acid (ALA) is synthesized by direct conjugation via ester bonds (FIG. 1). The nanoprodrug contains two antioxidant components, α-tocopherol and α-lipoic acid. α-Tocopherol serves as structural matrix and lipid-soluble antioxidant and α-lipoic acid in the triptolide prodrug serves as molecular switch. α-Tocopherol is the most relevant form of vitamin E and believed to be the most potent lipid-soluble antioxidant that can break the propagation of the free lipid radical chain reaction in the biological membrane. As a structural matrix, α-tocopherol reduced the size and increased chemical and physical stability of nanoprodrugs. α-Lipoic acid moiety of the triptolide prodrug scavenges a number of ROS, resulting in increased hydrophilicity and structural change, leading to enhanced prodrug activation.

In our previous patent applications and publications, inventors demonstrated that the α-lipoic acid moiety efficiently scavenged ROS, leading to accelerated destabilization of the nanoprodrug and increased prodrug activation, suggesting that the nanoprodrug is activated preferably in an oxidative environment, including the highly inflammatory tumor microenvironment.

### Synthesis of Triptolide Prodrug TPL-ALA

The synthesis of TPL-ALA by the coupling of triptolide (TPL) and α-lipoic acid (ALA) via ester bond was performed as follows. TPL (3.6 g, 10 mmol) and ALA (3.1 g, 15 mol) were placed in 50 mL of anhydrous dichloromethane (DCM) and reacted with 4-(dimethylamino)-pyridine (DMAP, 1.83 g, 15 mmol) in the presence of a molecular sieve (Fluka, 3 A, 10-20 mesh beads) for 10 min at room temperature. N-(3-Dimethylaminopropyl)-N-ethylcarbodiimide hydrochloride (EDCI, 2.9 g, 15 mmol) was added and the reaction mixture was stirred for 12 h at room temperature in the dark. The reaction mixture was filtered, and the solvent was evaporated until dryness and the residue was dissolved in acetone (50 mL) and insoluble matter was removed by filtration. H₂O (50 mL) was added to the clear acetone solution and the precipitate was collected and dried under vacuum. The precipitate was dissolved in acetone (50 mL). The solution was stored at −20° C. overnight and the insoluble matter was removed by centrifugation (20000×g for 10 min) The solvent was evaporated to dryness to yield the triptolide prodrug TPL-ALA. The HPLC analysis was performed with _{C18} RP column under isocratic condition with 50% acetonitrile containing 0.1% trifluoroacetic acid (TFA) at a flow rate of 1 mL/min. The compounds were detected using UV detector at 210 nm.

Additional antioxidant, anti-inflammatory, and anticancer derivatives of triptolide for nanoprodrug preparation:

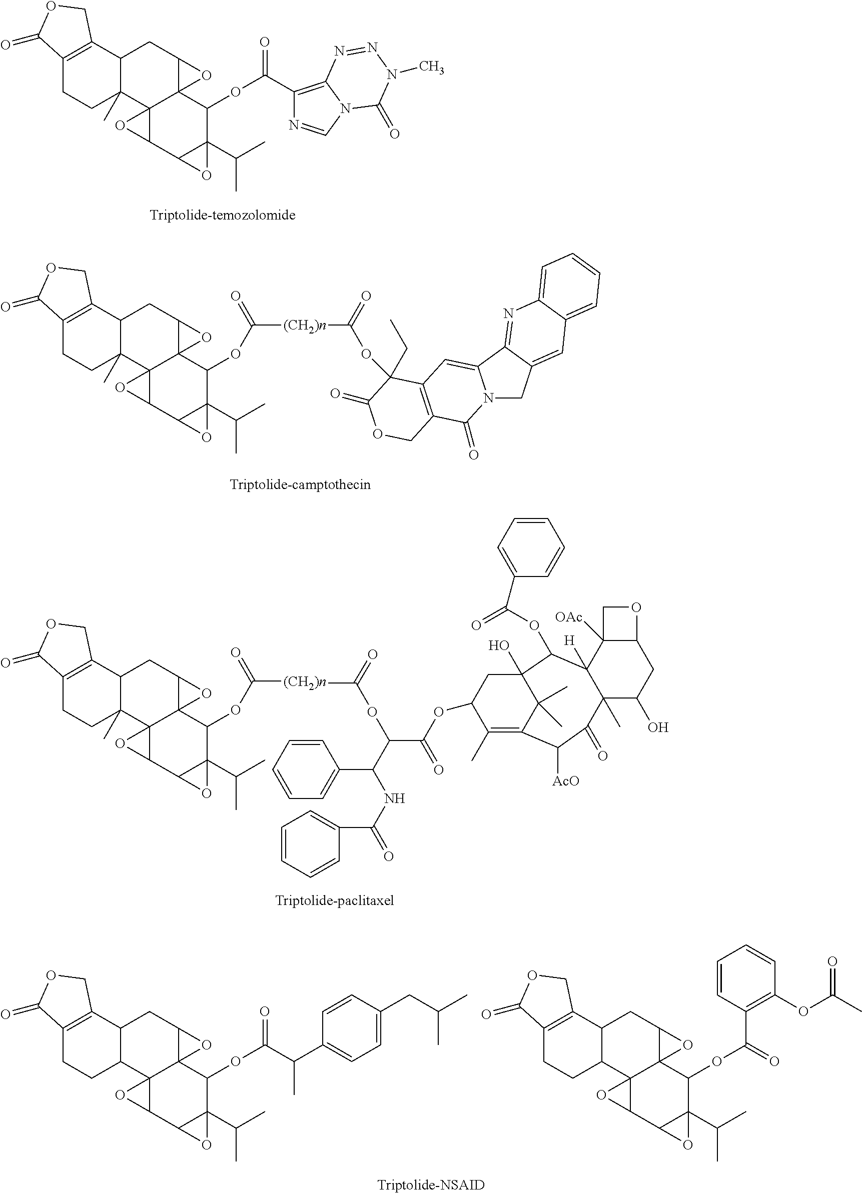

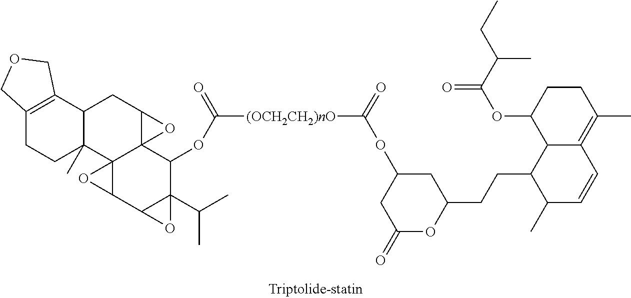

The inventive anti-immunosuppressive compounds and corresponding nanoprodrugs uses the nanoprodrug concept whereby two therapeutically active compounds are conjugated via biodegradable bond and transformed into reactive nanoprodug system. The presence of triptolide prodrug makes the nanoprodrug anti-immunosupopressive and thus augments the antinflammatory and anticancer efficacy of the second component in the nanoprodrug. This combination results in a synergistic effect in cancer treatment.

Various embodiments of the invention are described above in the Detailed Description. While these descriptions directly describe the above embodiments, it is understood that those skilled in the art can conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications can be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

1. A compound of formula D-I or D-II: 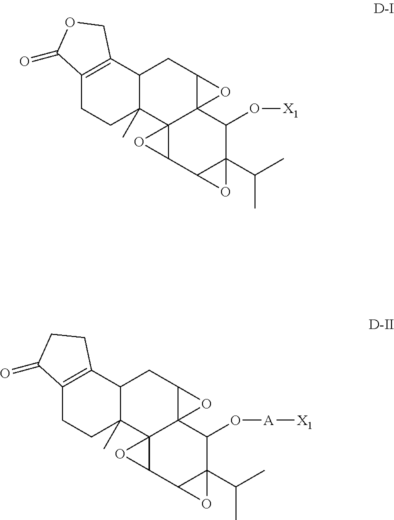
wherein
X1 is a antioxidant, an anti-inflammatory or an anticancer agent; and
A is selected from the group consisting of branched and unbranched alkyl, branched and unbranched alkenyl, branched and unbranched alkynyl, heteroatom-containing branched and unbranched alkyl, heteroatom-containing branched and unbranched alkenyl, heteroatom-containing branched and unbranched alkynyl, aryl, cyclic aliphatic, cyclic aromatic, heterocyclic, and aromatic heterocyclic groups.

2. The compound of claim 1, wherein X1 is selected from the group consisting of camptothecin, camptothecin analogs, temozolomide, temozolomide analogs, nonsteroidal anti-inflammatory drug (NSAID), statin, alpha-lipoic acid and chemotherapeutic agents.

3. The compound of claim 1, wherein the compound is of formula D-I.

4. The compound of claim 3, wherein the compound of formula D-I is selected from the group consisting of:

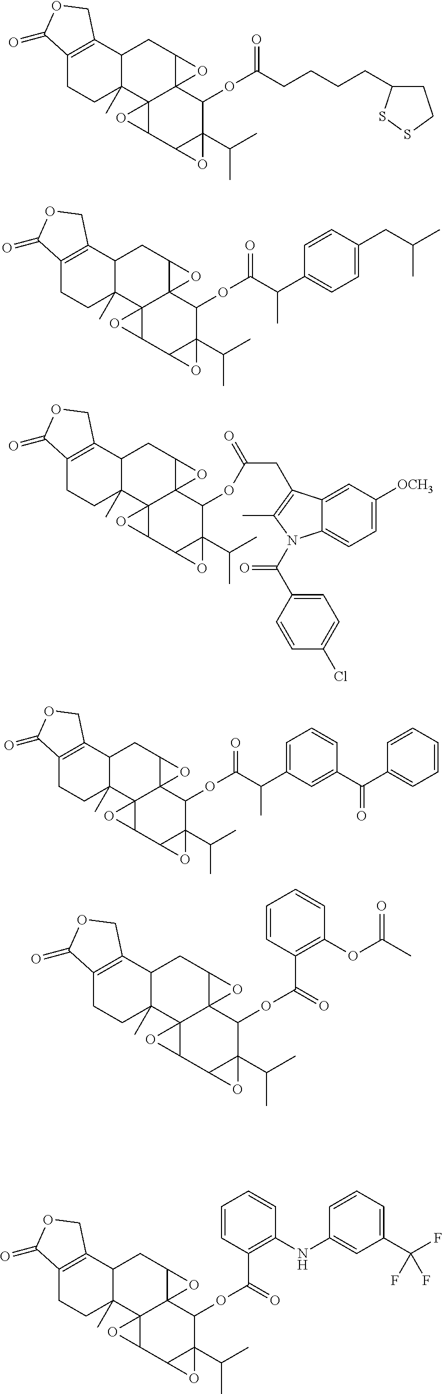 [SMILES: CC(C)C12OC1C1OC13C1(C)CCC4=C(COC4=O)C1CC1OC13C2OC(=O)CCCCC1CCSS1 ; CC(C)Cc1ccc(C(C)C(=O)OC2C3(C(C)C)OC3C3OC34C3(C)CCC5=C(COC5=O)C3CC3OC324)cc1 ; COc1ccc2c(c1)c(CC(=O)OC1C3(C(C)C)OC3C3OC34C3(C)CCC5=C(COC5=O)C3CC3OC314)c(C)n2C(=O)c1ccc(Cl)cc1 ; CC(C(=O)OC1C2(C(C)C)OC2C2OC23C2(C)CCC4=C(COC4=O)C2CC2OC213)c1cccc(C(=O)c2ccccc2)c1 ; CC(=O)Oc1ccccc1C(=O)OC1C2(C(C)C)OC2C2OC23C2(C)CCC4=C(COC4=O)C2CC2OC213 ; CC(C)C12OC1C1OC13C1(C)CCC4=C(COC4=O)C1CC1OC13C2OC(=O)c1ccccc1Nc1cccc(C(F)(F)F)c1]

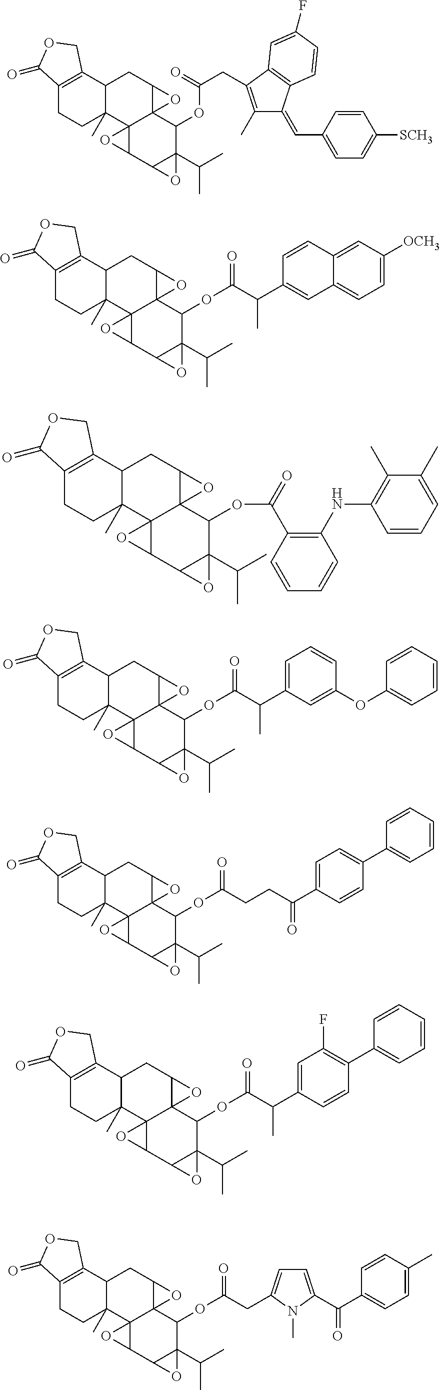

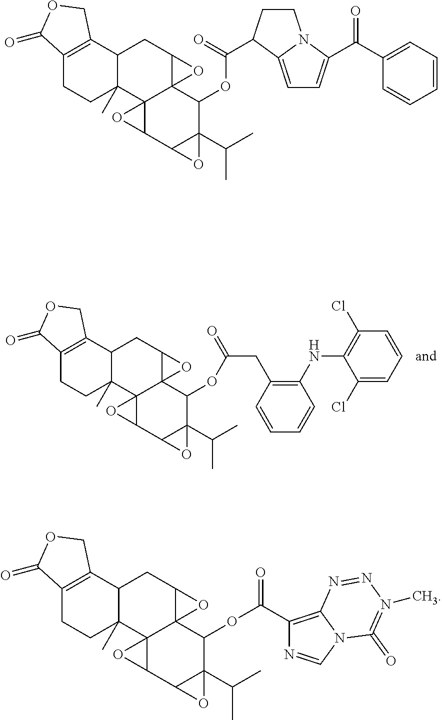

5. The compound of claim 1, wherein the compound is of formula D-II.

6. The compound of claim 5, wherein the compound of formula D-II is selected from the group consisting of: 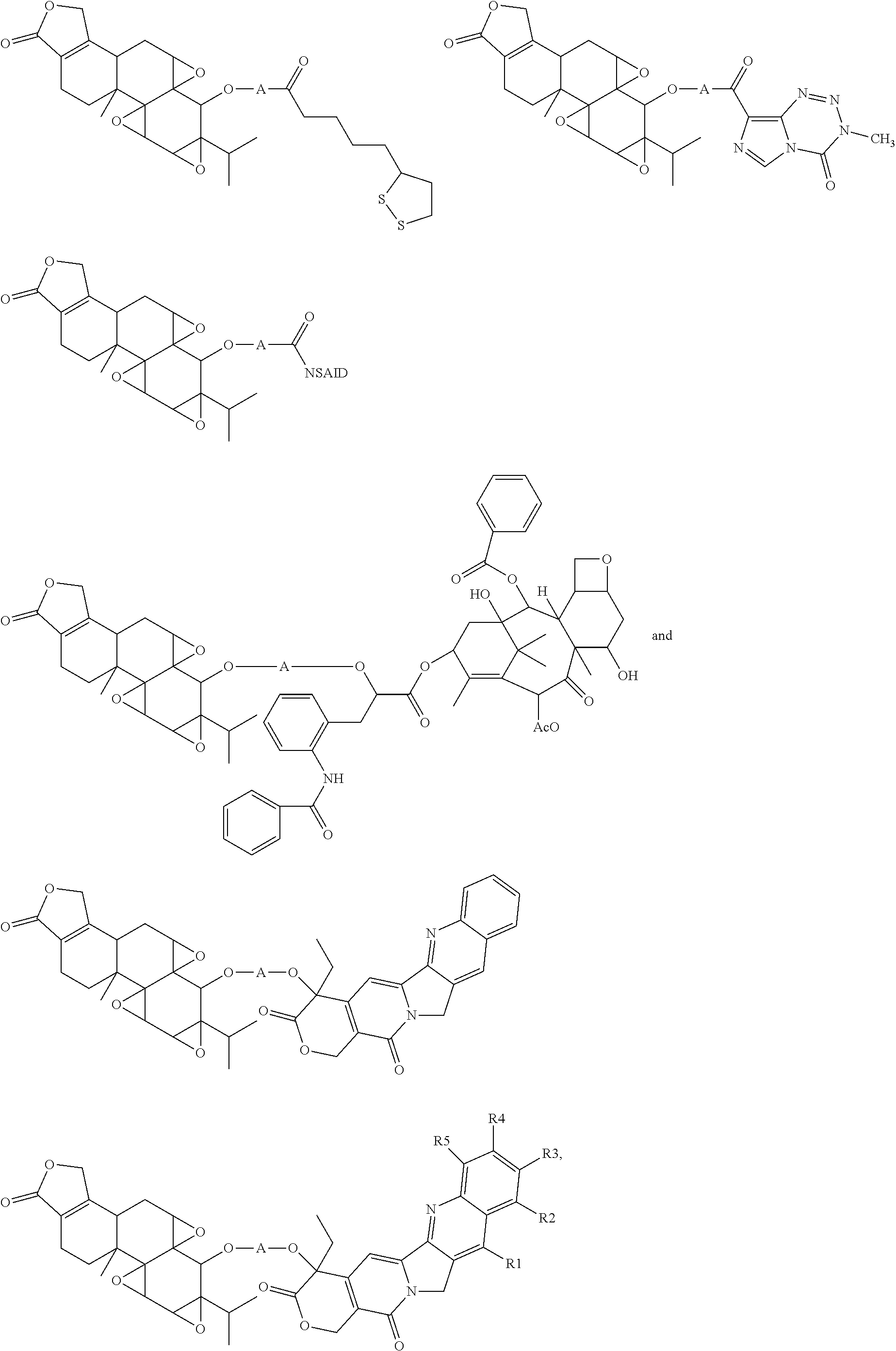
wherein R1, R2, R3, R4, and R5 are independently selected from the group consisting of hydrogen, alkyl, aryl, acyl, cycloaliphatic, aralkyl, acyl and hydroxyl, and can each optionally contain a hetero atom; and

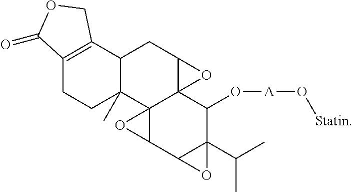

7. The compound of claim 6, wherein the compound of formula D-II is selected from the group consisting of 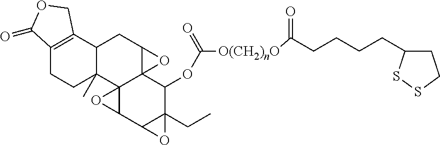
wherein n is an integer of 2-12, 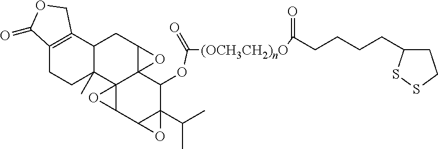
wherein n is an integer of 1-6, and 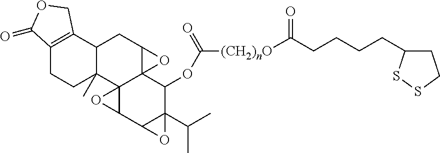
wherein n is an integer of 1-12.

8. The compound of claim 5, wherein the compound of formula D-II is selected from the group consisting of: 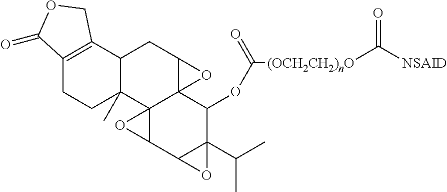
wherein n is an integer of 1-6, 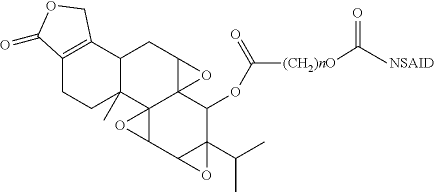
wherein n is an integer of 1-12, and 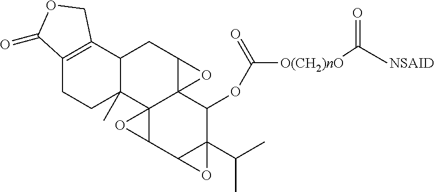
wherein n is an integer of 1-12, 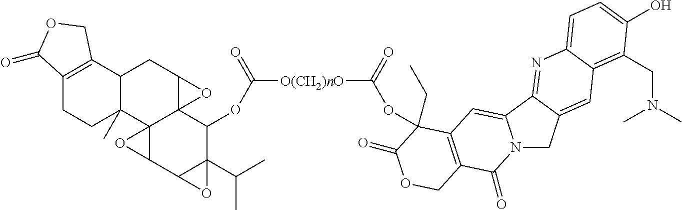
wherein n is an integer of 2-12, 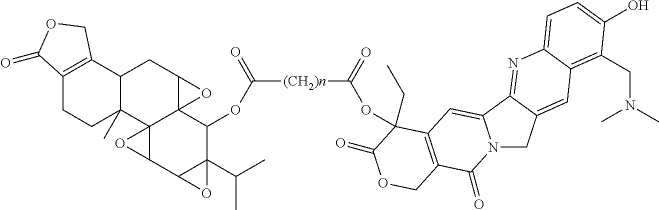
wherein n is an integer of 2-12, 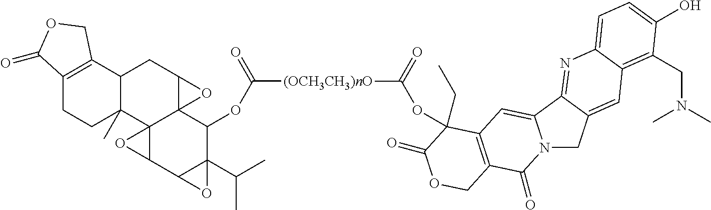
wherein n is an integer of 1-6, 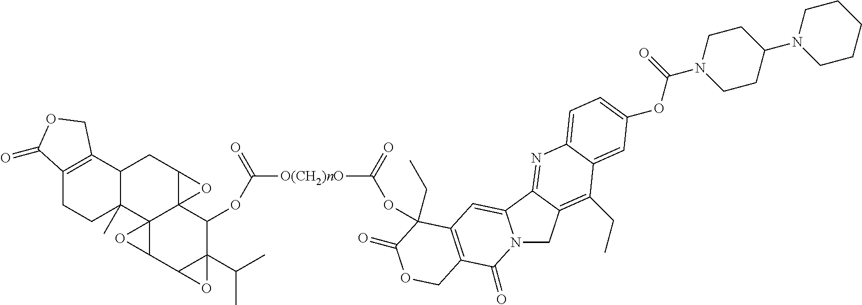
wherein n is an integer of 2-12, 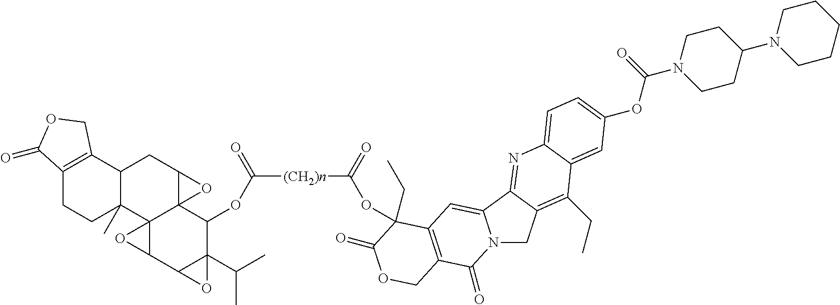
wherein n is an integer of 2-12, and 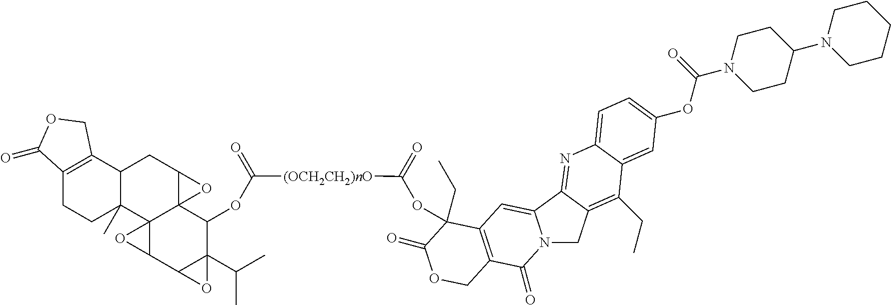
wherein n is an integer of 1-6.

9. The compound of claim 6, wherein the compound of formula D-II is: 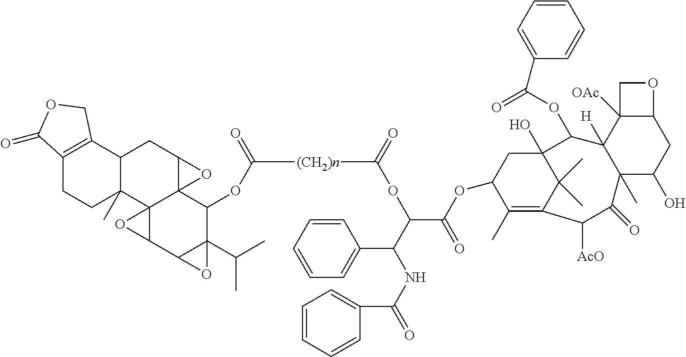
wherein n is an integer of 2-12.

10. The compound of claim 5, wherein formula D-II is selected from the group consisting of: 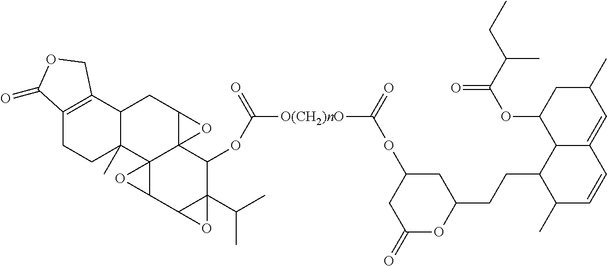
wherein n is an integer of 2-12, 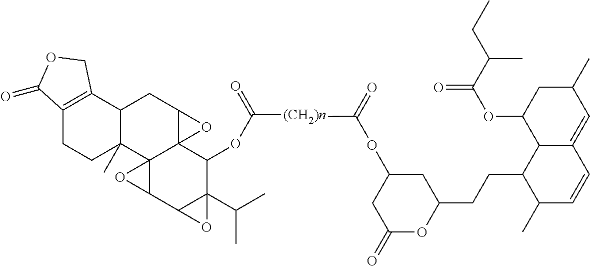
wherein n is an integer of 2-12, 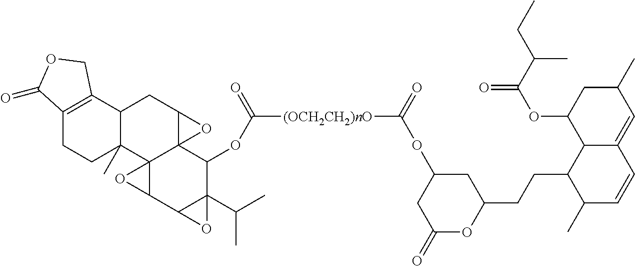
wherein n is an integer of 1-6, 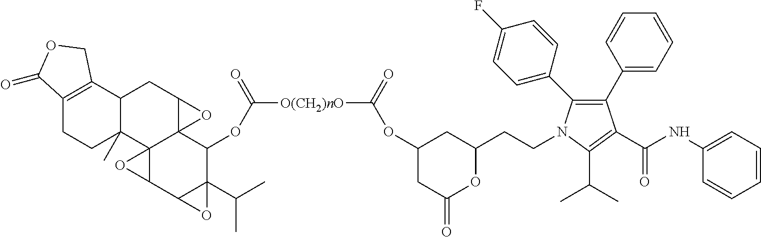
wherein n is an integer of 1-12, 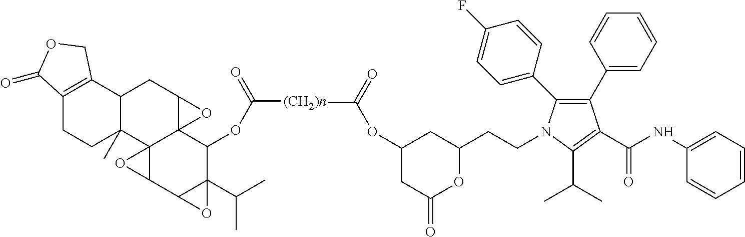
wherein n is an integer of 1-12, and 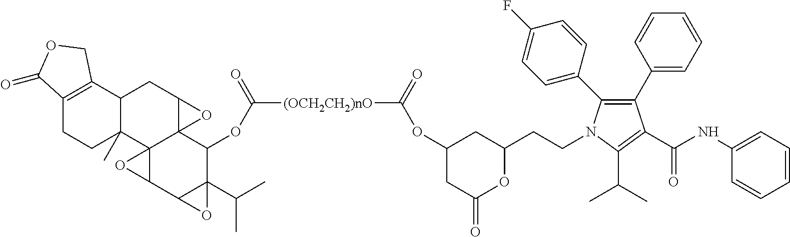
wherein n is an integer of 1-6.

11. A pharmaceutical composition comprising a compound of claim 1 and a pharmaceutically acceptable carrier or excipient.

12. A nanosphere comprising a compound of claim 1.

13. The nanosphere of claim 12, wherein the nanosphere further comprises an antioxidant.

14. The nanosphere of claim 13, wherein the antioxidant is tocopherol or a derivative thereof.

15. The nanosphere of claim 12, wherein the nanosphere further comprises a c compound of formula A-IV or A-V.

16. The nanosphere of claim 12, wherein the nanosphere further comprises an amphiphilic spacer.

17. The nanosphere of claim 16, wherein the amphiphilic spacer comprises a chemically active group selected from the group consisting of thiol, amine, carboxylic acid, carboxylic acid NHS ester, maleimide, hydrazine, ketone, aledehyde, and combinations thereof

18. The nanosphere of claim 16, wherein the amphiphilic spacer is an alkylthiol or an alkylamine.

19. The nanosphere of claim 16, wherein the amphiphilic spacer is 1-octadecanethiol.

20. The nanosphere of claim 12, wherein the nanosphere further comprises a polymer.

21. The nanosphere of claim 20, wherein the polymer is poly(lactide-co-glycolide) (PLGA).

22. The nanosphere of claim 12, wherein the nanosphere further comprises a therapeutic agent.

23. The nanosphere of claim 22, wherein the therapeutic agent is selected from the group consisting of: a chemotherapeutic agent, statin, nonsteroidal anti-inflammatory drug (NSAID), and combinations thereof.

24. The nanosphere of claim 23, wherein the chemotherapeutic agent is selected from the group consisting of actinomycin, all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vinblastine, vincristine, vindesine, vinorelbine, and any combinations thereof.

25. The nanosphere of claim 12, wherein the therapeutic agent is conjugated to a hydrophilic spacer, a hydrophobic spacer, an amphiphilic spacer, or an amphiphilic polymer.

26. The nanosphere of claim 12, wherein the nanosphere further comprises an imaging agent.

27. The nanosphere of claim 26, wherein the imaging agent is conjugated to a hydrophilic spacer, a hydrophobic spacer, an amphiphilic spacer, or an amphiphilic polymer.

28. The nanosphere of claim 26, wherein the imaging agent is selected from the group consisting of fluorescent molecules, radioisotopes, nucleotide chromophores, enzymes, substrates, chemiluminescent moieties, magnetic particles, bioluminescent moieties, antibody against a protein expressed or overexpressed in cancer, and combinations thereof.

29. The nanosphere of claim 12, further comprising a compound selected from the group consisting of: a multiple α-lipoic acid-containing hydrophobic compound, α-tocopherol, a nonsteroidal anti-inflammatory drug (NSAID) derivative, an antioxidant derivative of camptothecin and/or camptothecin analogs, a statin lactone derivative, paclitaxel, and any combinations thereof.

30. A pharmaceutical composition comprising a nanosphere of claim 12 and a pharmaceutically acceptable carrier or excipient.

31. A method of delivering a nanosphere to a tumor or cancer in a subject, comprising: administering a therapeutically effective amount of a nanosphere of claim 12 to the subject.

32-51. (canceled)

52. A method of detecting or diagnosing cancer in a subject in need thereof comprising:
administering an effective amount of a nanosphere of claim 12 to the subject, wherein the nanosphere further comprises an imaging agent; and
imaging the subject to detect or diagnose the cancer.

53-68. (canceled)

69. A method of treating cancer in a subject in need thereof, comprising: administering a therapeutically effective amount of a compound of claim 1 to the subject to treat cancer.

70. (canceled)

71. A method of treating cancer in a subject in need thereof, comprising: administering a therapeutically effective amount of a nanosphere of claim 12 to the subject to treat cancer.

72-89. (canceled)
